SEQUENCE LISTING (33 sequences)

SEQ ID NO: 1 - DNA, 44 bp, Artificial Sequence
  Synthetic
cccaggtcccacgcgtatgcagaacagtcacagcggagtgaatc

SEQ ID NO: 2 - DNA, 51 bp, Artificial Sequence
  Sythetic
ggtagaattcacgcgtttactgtaatcttggccagtattgagacatatcag

SEQ ID NO: 3 - DNA, 40 bp, Artificial Sequence
  Synthetic
cccaggtcccacgcgtatgctgttccacagtctgtcgggc

SEQ ID NO: 4 - DNA, 50 bp, Artificial Sequence
  Synthetic
ggtagaattcacgcgttcattagaaaaggttggtaagagtcgtttgtgag

SEQ ID NO: 5 - DNA, 47 bp, Artificial Sequence
  Synthetic
cccaggtcccacgcgtatgatgtcttatcttaagcaaccgccttacg

SEQ ID NO: 6 - DNA, 45 bp, Artificial Sequence
  Synthetic
ggtagaattcacgcgttcacaaaacctggaatttccacgaggatg

SEQ ID NO: 7 - DNA, 43 bp, Artificial Sequence
  Synthetic
cccaggtcccacgcgtatgaatctcctggaccccttcatgaag

SEQ ID NO: 8 - DNA, 41 bp, Artificial Sequence
  Synthetic
ggtagaattcacgcgttcaaggtcgagtgagctgtgtgtag

SEQ ID NO: 9 - DNA, 47 bp, Artificial Sequence
  Synthetic
cccaggtcccacgcgtatgctggaaatgctagaatataatcactatc

SEQ ID NO: 10 - DNA, 40 bp, Artificial Sequence
  Synthetic
ggtagaattcacgcgttcagtgacaccgacgggagaaagg

SEQ ID NO: 11 - DNA, 40 bp, Artificial Sequence
  Synthetic
cccaggtcccacgcgtatggtattccgctcccccctagac

SEQ ID NO: 12 - DNA, 44 bp, Artificial Sequence
  Synthetic
ggtagaattcacgcgtttatacatcacattccgagtcgctggag

SEQ ID NO: 13 - DNA, 43 bp, Artificial Sequence
  Synthetic
cccaggtcccacgcgtatgggaccactgacatttagggatgtg

SEQ ID NO: 14 - DNA, 51 bp, Artificial Sequence
  Synthetic
ggtagaattcacgcgttcaggtttgtagtttctctttagtataatttgagg

SEQ ID NO: 15 - DNA, 45 bp, Artificial Sequence
  Synthetic
cccaggtcccacgcgtatgatggttcagcgactgggactcatttc

SEQ ID NO: 16 - DNA, 43 bp, Artificial Sequence
  Synthetic
ggtagaattcacgcgtttagccttcggtgtagacagaggagag

SEQ ID NO: 17 - DNA, 41 bp, Artificial Sequence
  Synthetic
cccaggtcccacgcgtatgaccctgagcactgagatgtccg

SEQ ID NO: 18 - DNA, 50 bp, Artificial Sequence
  Synthetic
ggtagaattcacgcgtctaacaattggaaatcctagcagtaaagttctcg

SEQ ID NO: 19 - DNA, 20 bp, Artificial Sequence
  Synthetic
gggacagcagagatccagtt

SEQ ID NO: 20 - DNA, 20 bp, Artificial Sequence
  Synthetic
tactaccaccgattgccctg

SEQ ID NO: 21 - DNA, 20 bp, Artificial Sequence
  Synthetic
ctttgccattaaccacaacc

SEQ ID NO: 22 - DNA, 20 bp, Artificial Sequence
  Synthetic
cttcctgactaggggaggag

SEQ ID NO: 23 - DNA, 20 bp, Artificial Sequence
  Synthetic
tgtcagggtagcgagtcttg

SEQ ID NO: 24 - DNA, 20 bp, Artificial Sequence
  Synthetic
tcaacctcccacactacagc

SEQ ID NO: 25 - DNA, 19 bp, Artificial Sequence
  Synthetic
agactgccttgggaaaagc

SEQ ID NO: 26 - DNA, 20 bp, Artificial Sequence
  Synthetic
ctctctgccctgttttgctc

SEQ ID NO: 27 - DNA, 20 bp, Artificial Sequence
  Synthetic
tcactcccacacaagtaggc

SEQ ID NO: 28 - DNA, 20 bp, Artificial Sequence
  Synthetic
cttccatttgtcacgtcctg

SEQ ID NO: 29 - DNA, 22 bp, Artificial Sequence
  Synthetic
tcttgggcaaaatgagaacaca

SEQ ID NO: 30 - DNA, 20 bp, Artificial Sequence
  Synthetic
agggacagcagagatccagt

SEQ ID NO: 31 - DNA, 20 bp, Artificial Sequence
  Synthetic
gggacctggtatctgaagga

SEQ ID NO: 32 - DNA, 20 bp, Artificial Sequence
  Synthetic
ggtacagtgcaggggaaaga

SEQ ID NO: 33 - DNA, 20 bp, Artificial Sequence
  Synthetic
agaggcatcggacatctcag

(19) United States
(12) Patent Application Publication
(10) Pub. No.: US 2017/0002319 A1
(43) Pub. Date: Jan. 5, 2017

(54) Master Transcription Factors Identification and Use Thereof

(71) Applicant: Whitehead Institute for Biomedical Research, Cambridge, MA (US)

(72) Inventors: Ana C. D'Alessio, Cambridge, MA (US); Tang Ihn Lee, Somerville, MA (US); Zi Peng Fan, Waltham, MA (US); Richard A. Young, Boston, MA (US)

(21) Appl. No.: 15/266,390

(22) Filed: Sep. 15, 2016

Related U.S. Application Data
(63) Continuation of application No. 15/154,259, filed on May 13, 2016, now abandoned.
(60) Provisional application No. 62/161,163, filed on May 13, 2015.
(60) Provisional application No. 62/242,454, filed on Oct. 16, 2015.

(51) Int. Cl. C12N 5/079; G06F 19/22; G06F 19/24; G06F 19/20

(52) U.S. Cl. CPC C12N 5/0621; G06F 19/20; G06F 19/22; G06F 19/24; C12N 2501/60; C12N 2506/09

(57) ABSTRACT

Provided herein are methods for identifying master transcription factors (TFs) in a cell type of interest and for transdifferentiation of a somatic cell, e.g., a fibroblast to the cell type of interest. Also provided herein are induced retinal pigment epithelium (iRPE) cell, master TFs therefor, methods for making iRPE cell, and methods and compositions for treating an ocular disease such as age-related macular degeneration.

### CROSS-REFERENCE TO RELATED APPLICATIONS

This application is a continuation of U.S. application Ser. No. 15/154,259, filed May 13, 2016, which claims priority to and the benefit of U.S. Provisional Application No. 62/161,163 filed May 13, 2015 and 62/242,454 filed Oct. 16, 2015, the disclosures of all of which applications are incorporated herein by reference in their entirety.

### GOVERNMENT SUPPORT

This invention was made with Government Support under Grant No. R01-HG002668 awarded by the National Human Genome Research Institute and Grant No. CA146445 awarded by the National Institutes of Health. The Government has certain rights in the invention.

### FIELD

The disclosure relates in general to methods for identifying master transcription factors (TFs) in a cell type of interest, or a cell in a first state, and transdifferentiation of a somatic cell, e.g., a fibroblast to the cell type of interest, or induction of the cell in the first state into a second state. The present disclosure also relates to an induced retinal pigment epithelium (iRPE) cell, master TFs therefor, methods for making iRPE cell, and methods and compositions for treating an ocular disease such as age-related macular degeneration.

### BACKGROUND

For some cell types, direct reprogramming can be achieved by ectopic expression of key transcription factors of the target cell type in cells of a different type (Buganim et al., 2013; Morris and Daley, 2013; Sancho-Martinez et al., 2012; Vierbuchen and Wernig, 2012; Yamanaka, 2012). Due to limited knowledge of the key factors for each cell type, however, it is not currently possible to obtain various clinically relevant cell types by this approach. The identification of such master transcription factors in all cell types might thus facilitate advances in direct reprogramming for clinically relevant cell types.

Accordingly, a need exists for methods of identifying master transcription factors that can induce transdifferentiation of somatic cells into a cell type of interest.

### SUMMARY

In one aspect, the present disclosure features a method of identifying master transcription factors of a query cell type, comprising:
providing gene expression data of a plurality of transcription factors for a query cell type;
relatively quantifying expression level and expression specificity of each transcription factor in the query cell type against a background gene expression profile assembled from a collection of cell types by using an entropy-based measure of Jensen-Shannon divergence (JSD), thereby generating a cell-type-specificity score for each transcription factor; and
ranking the plurality of transcription factors based on their corresponding cell-type-specificity scores, wherein top ranked transcription factors are identified as master transcription factors of the query cell type.

In some embodiments, in the providing step, the gene expression data is selected from one or more of: gene expression profiling by microarray or sequencing, non-coding RNA profiling by microarray or sequencing, chromatin immunoprecipitation profiling by microarray or sequencing, genome methylation profiling by microarray or sequencing, genome variation profiling by array, single nucleotide polymorphism array, serial analysis of gene expression, and/or protein array. In some embodiments, a plurality of disparate sets of gene expression data are provided.

The method in some embodiments can further include comparing the plurality of disparate sets of gene expression data by pair-wise Pearson correlation, grouping the plurality of disparate sets into subclusters using hierarchical clustering, analyzing the subclusters in a modular fashion, and removing subclusters consisting of data sets that have Pearson correlation coefficients less than 0.7 compared to other data sets.

In some embodiments, the ranking step further comprises calculating rank product-based scores for each set of gene expression data that is retained after the removing step.

In some embodiments, the quantifying step uses an algorithm which:
assumes an idealized pattern where an ideal master transcription factor is expressed to a high level in the query cell type and not expressed in any other cell type;
compares the observed pattern of an actual transcription factor with the idealized pattern; and
generates the cell-type-specificity score based on how well the observed pattern matches with the idealized pattern.

In some embodiments, the method further includes:
creating two same-sized, discrete, first and second probability vectors to represent the observed pattern and the ideal pattern, respectively; wherein for the observed pattern, the first probability vector is formed by values from the gene expression data of the query cell type and the background gene expression profile, and elements in the first probability vector are divided by the sum of the elements so that the normalized vector sums to 1; wherein for the idealized pattern, the second probability vector is formed by a value of 1 at a position equivalent to that of the query cell type and zeroes at all other positions; and
calculating a distance metric between the first and second vectors using JSD, thereby generating the cell-type-specificity score.

In certain embodiments, the background gene expression profile is prepared by a method comprising the steps of:
collecting a background dataset comprising expression datasets of different cell and tissues types,
normalizing expression profiles of the expression datasets, and
balancing the background dataset.

In the above collecting step, the expression datasets can be gathered from Human Body Index collection of expression datasets. In the normalizing step, the expression profiles can be processed and normalized to generate Affymetrix MAS5-normalized probe set values. In some embodiments, the balancing step comprises clustering the expression profiles in the background dataset by similarity, and choosing from clusters of highly similar expression profiles a single representative profile while removing other profiles from the background dataset.

In some embodiments, top 20 or less ranked, top 10 or less ranked, or top 5 or less ranked transcription factors are identified as master transcription factors of the query cell type.

In certain embodiments, the query cell type and the collection of cell types are from human.

Also provided herein, in another aspect, is a method of transdifferentiating a cell of a first somatic cell type to a cell of a second somatic cell type, comprising identifying master transcription factors for said second somatic cell type according to the methods disclosed herein, and ectopically expressing one or more of the identified master transcription factors in a cell of said first somatic cell type. In some embodiments, the cell of the first somatic cell type is from a patient in need of cell or tissue replacement therapy with cells of the second somatic cell type. In certain embodiments, the second somatic cell type is selected from those listed in Tables 1 and 2, and the master transcription factors for each cell type are one or more of the top 10 scoring transcription factors listed in Tables 1 and 2 or a subset thereof. In some embodiments, one or more additional transcription factors such as the top 11-20 listed in Table 1, or those listed in Table 1A can be additionally ectopically expressed in the cell of the first somatic cell type.

Another aspect relates to a method of inducing a cell in a first state into a second state, comprising identifying master transcription factors for said cell in the first state according to the method described herein, and altering expression level of one or more of the identified master transcription factors to induce the cell into the second state. In some embodiments, the first state is a first somatic cell type and the second state is a second somatic cell type. The method can further include identifying master transcription factors for said second somatic cell type, and ectopically expressing one or more of the identified master transcription factors in a cell of said first somatic cell type. In some embodiments, the cell of the first somatic cell type is from a patient in need of cell or tissue replacement therapy with cells of the second somatic cell type. In certain embodiments, the second somatic cell type is selected from those listed in Tables 1 and 2, and the master transcription factors for each cell type are the top 20 or top 10 scoring transcription factors listed in Tables 1 and 2, or a subset thereof. In some embodiments, the first state is an undesirable state and wherein altering expression level comprises reducing or inhibiting expression thereby removing the cell from the first state.

Also provided herein is a cell engineered to ectopically expressing at least 1, at least 2, at least 3, at least 4, at least 5, at least 6, at least 7, at least 8, at least 9 or at least 10 of the top 20 scoring transcription factors listed in Table 1. The cell can be a fibroblast in some embodiments. The cell can, in certain embodiments, further include one or more ectopically expressed transcription factor selected from Table 1A.

In a further aspect, provided herein is a method of transdifferentiating a somatic cell into an induced retinal pigment epithelium (iRPE) cell, comprising increasing expression of at least two, at least three, or at least four of PAX6, LHX2, OTX2, SOX9, MITF, SIX3, ZNF92, GLIS3, C11orf9 and FOXD1, or a variant of any one or more of the foregoing, in a somatic cell that is not retinal pigment epithelium cell. In some embodiments, the method further includes ectopically expressing OTX2, SIX3, GLIS3, and at least one of PAX6, LHX2, SOX9, MITF, ZNF92, C11orf9 and FOXD1, or a variant of any one or more of the foregoing in the somatic cell. The method can further include increasing expression of PAX6, OTX2, MITF, SIX3, GLIS3 and FOXD1, or a variant of any one or more of the foregoing, or increasing expression of PAX6, OTX2, MITF and SIX3, or a variant of any one or more of the foregoing. The somatic cell in some embodiments is a fibroblast cell. The somatic cell can be present in vitro or ex vivo. The method somatic cell in some embodiments can be obtained from a subject in need of RPE cell replacement therapy, where for example, the subject has age-related macular degeneration, macular edema (including diabetic macular edema), proliferative vitreoretinopathy, branch and central retinal vein occlusion, retinitis pigmentosa, retinal detachment, diabetic retinopathy, retinal degeneration, vascular retinopathy, uveitis, AIDS-related retinitis, choroidal and retinal neovascularization, or macular telangiectasia. In some embodiments, the iPRE cell exhibits one or more characteristics of an endogenous RPE cell, selected from a cobblestone sheet colony morphology, gene expression signature, phagocytosis of photoreceptor rod outer segments, formation of a barrier for ion transport, and polarized growth factor secretion.

Also provided herein is an induced retinal pigment epithelium (iRPE) cell, comprising at least two, at least three, or at least four of ectopically expressed PAX6, LHX2, OTX2, SOX9, MITF, SIX3, ZNF92, GLIS3, C11orf9 and FOXD1, or a variant of any one or more of the foregoing, in a somatic cell that is not retinal pigment epithelium cell. The induced iRPE can, in some embodiments, include ectopically expressed OTX2, SIX3, GLIS3, and at least one of PAX6, LHX2, SOX9, MITF, ZNF92, C11orf9 and FOXD1, or a variant of any one or more of the foregoing; include ectopically expressed PAX6, OTX2, MITF, SIX3, GLIS3 and FOXD1, or a variant of any one or more of the foregoing; or include ectopically expressed PAX6, OTX2, MITF and SIX3, or a variant of any one or more of the foregoing. The induced iRPE can be for use in a treatment of an ocular disease selected from age-related macular degeneration, macular edema (including diabetic macular edema), proliferative vitreoretinopathy, branch and central retinal vein occlusion, retinitis pigmentosa, retinal detachment, diabetic retinopathy, retinal degeneration, vascular retinopathy, uveitis, AIDS-related retinitis, choroidal and retinal neovascularization, or macular telangiectasia.

In another aspect, provided herein is a method of treating an ocular disease, comprising administering to a patient in need thereof the induced iRPE disclosed herein.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIGS. 1A-1D. A general approach to identify candidate master transcription factors in human cells.

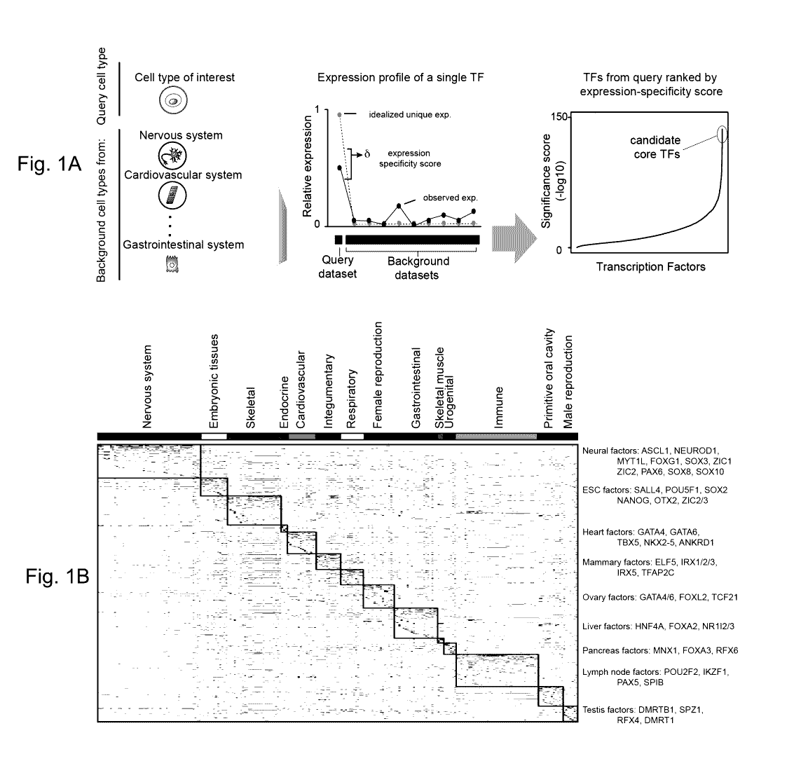

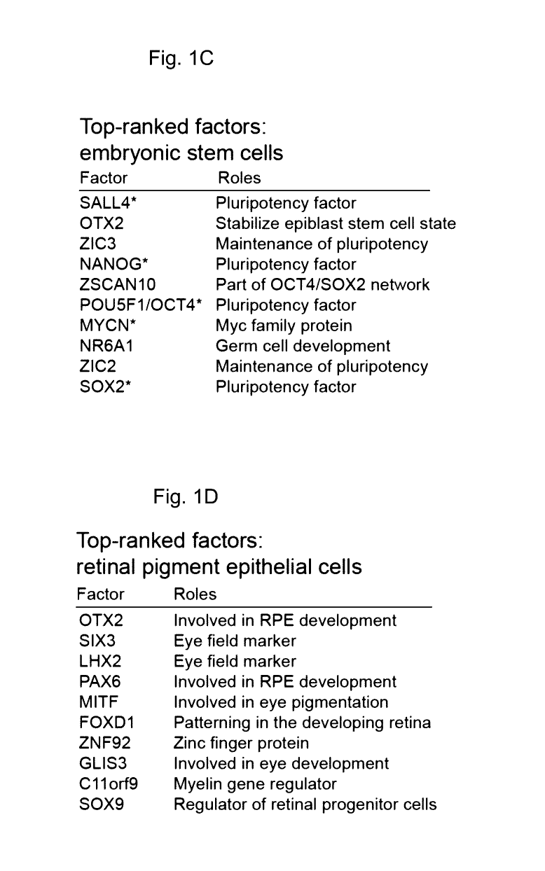

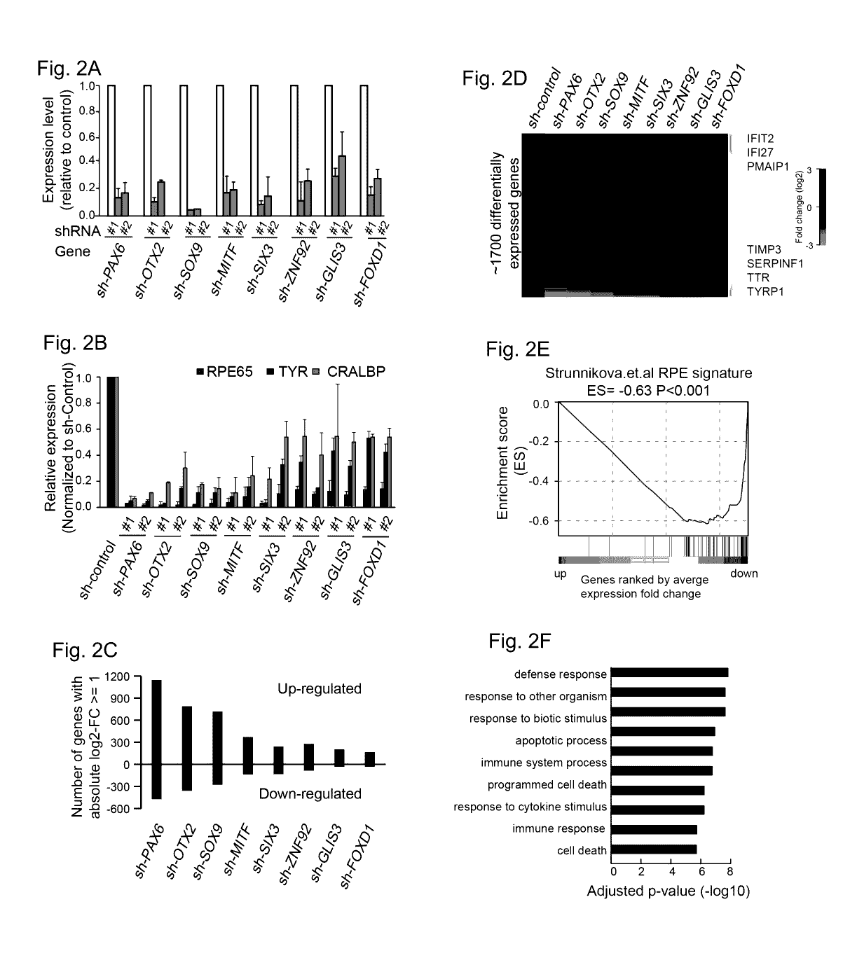

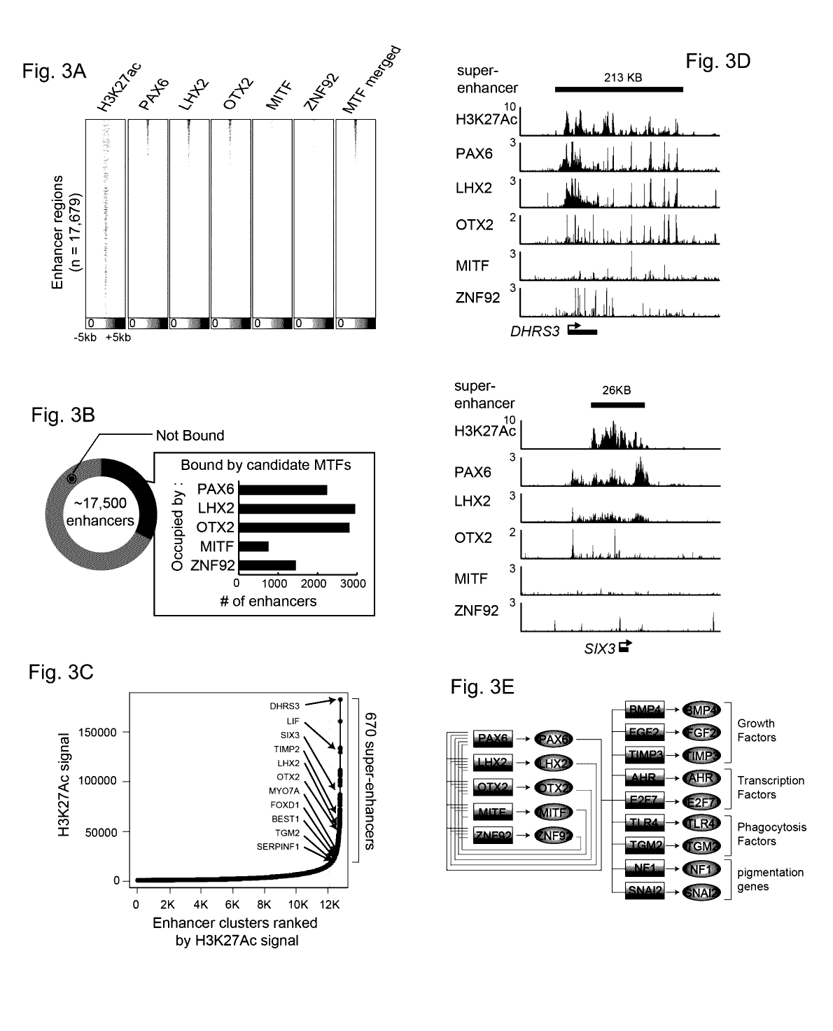

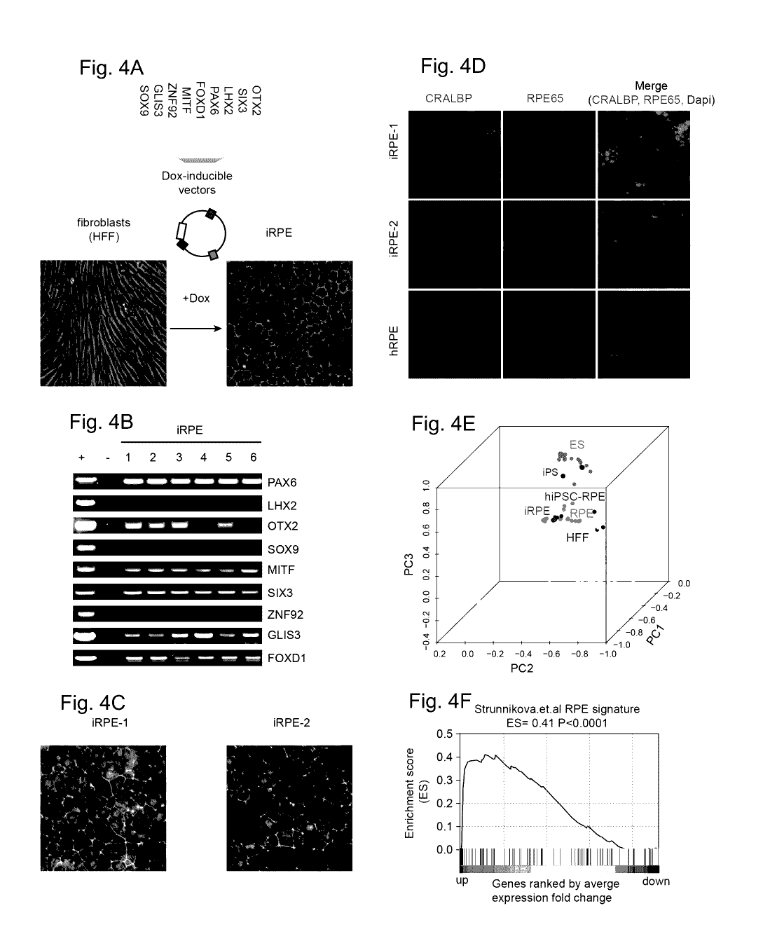

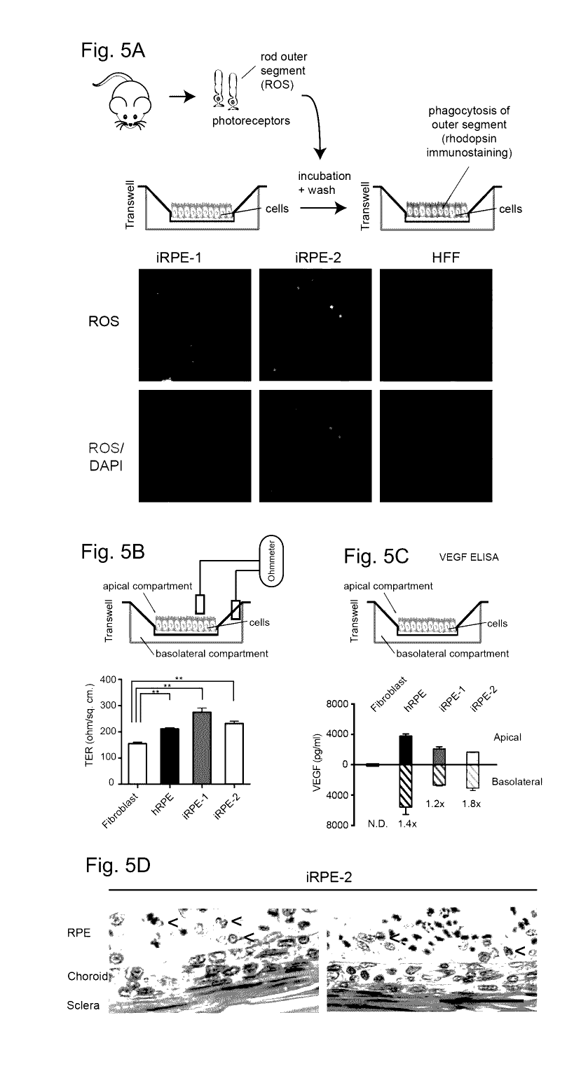

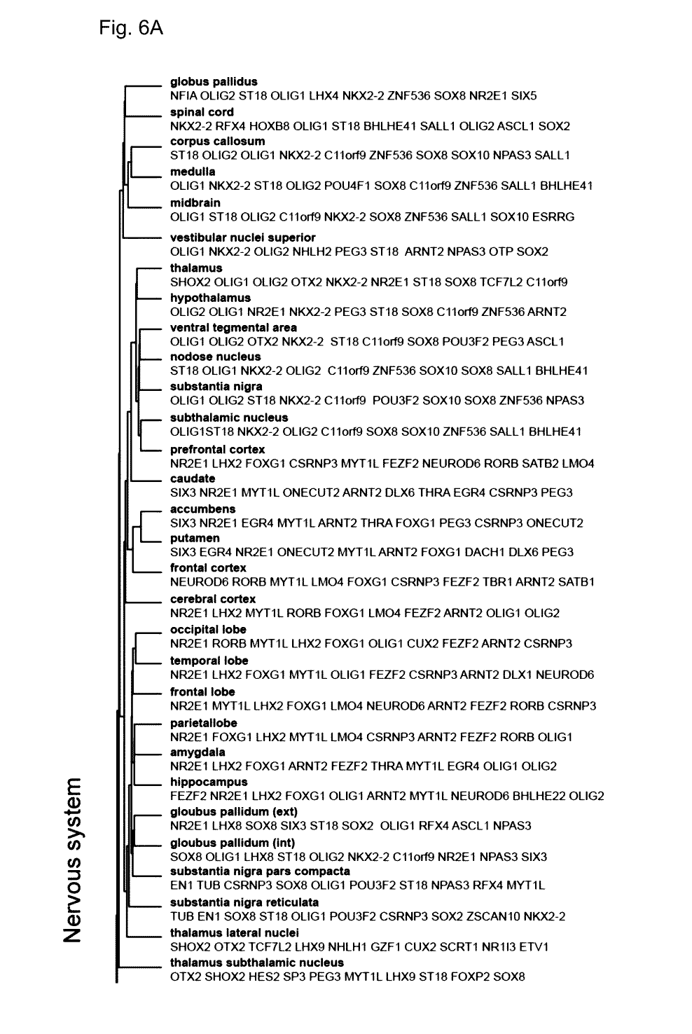

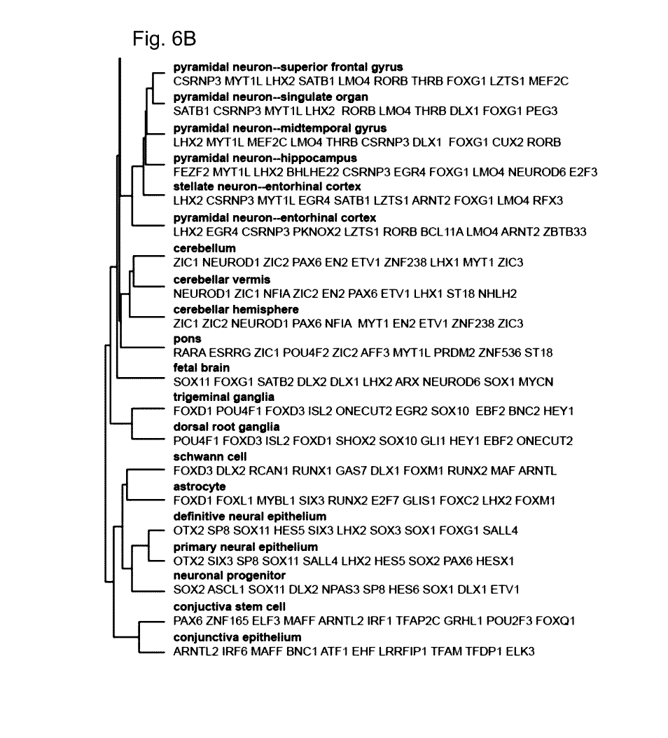

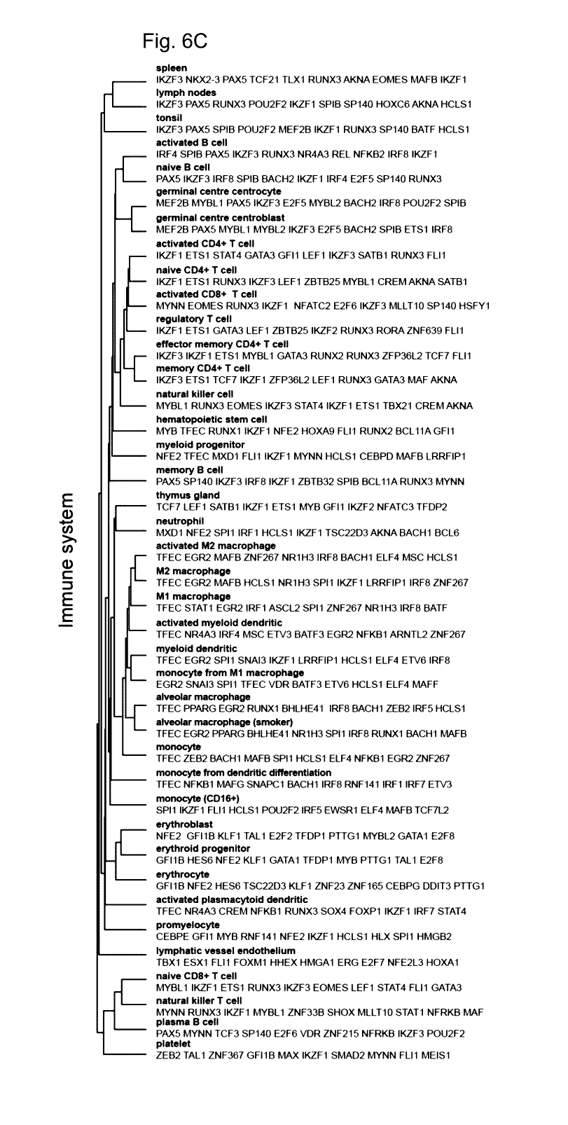

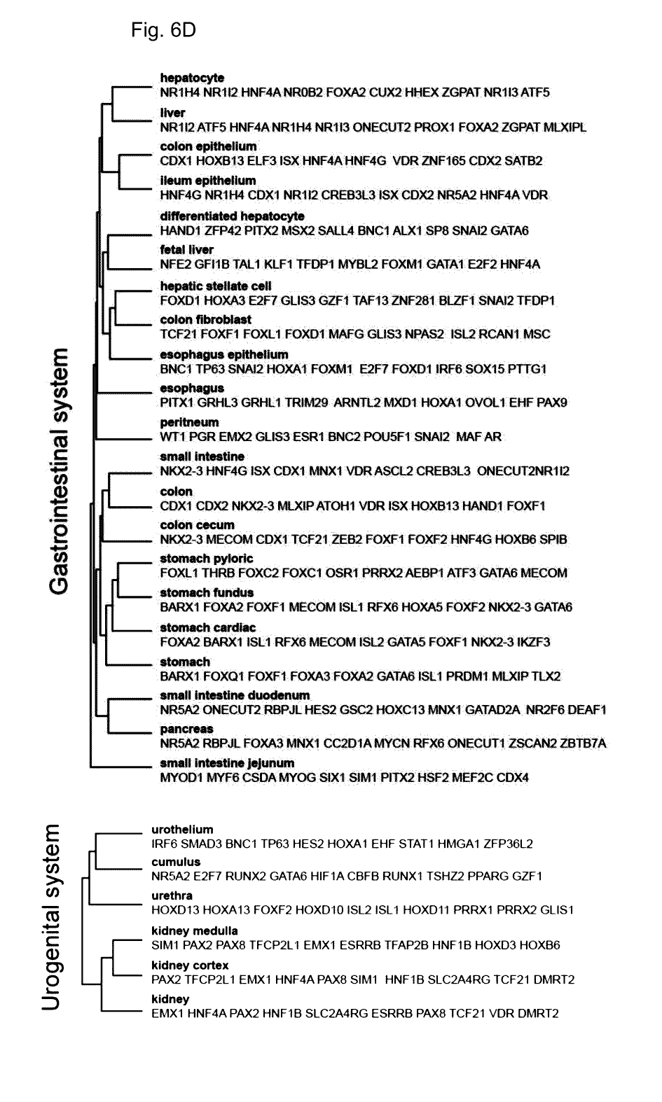

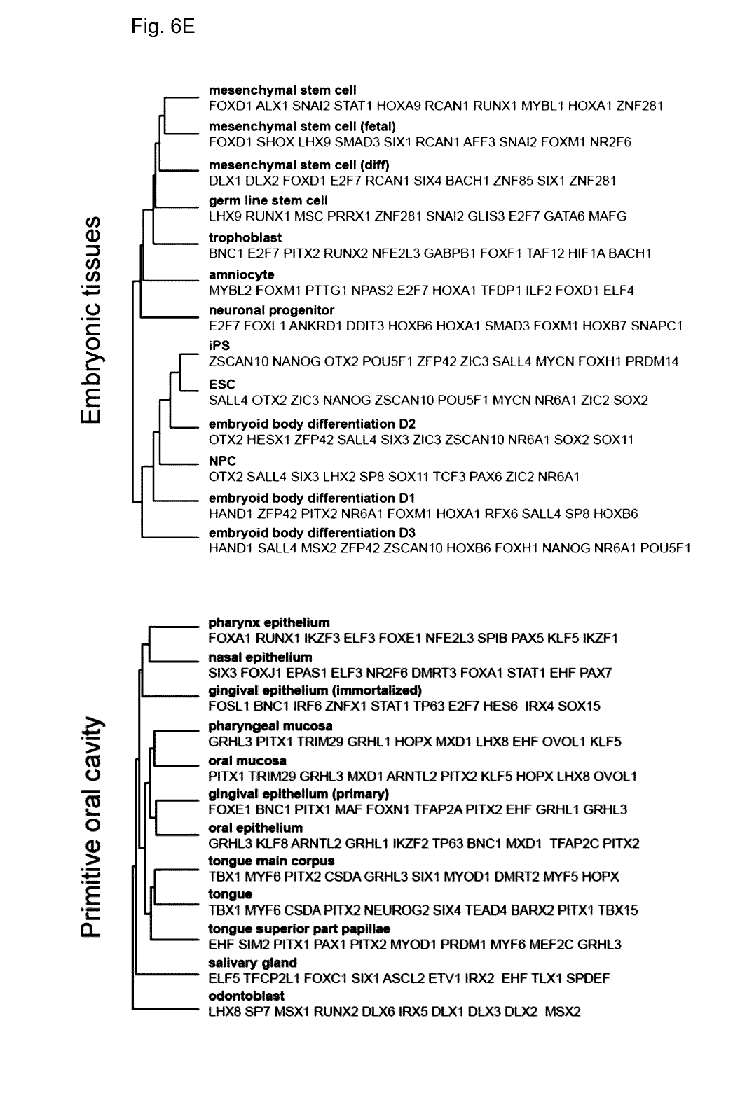

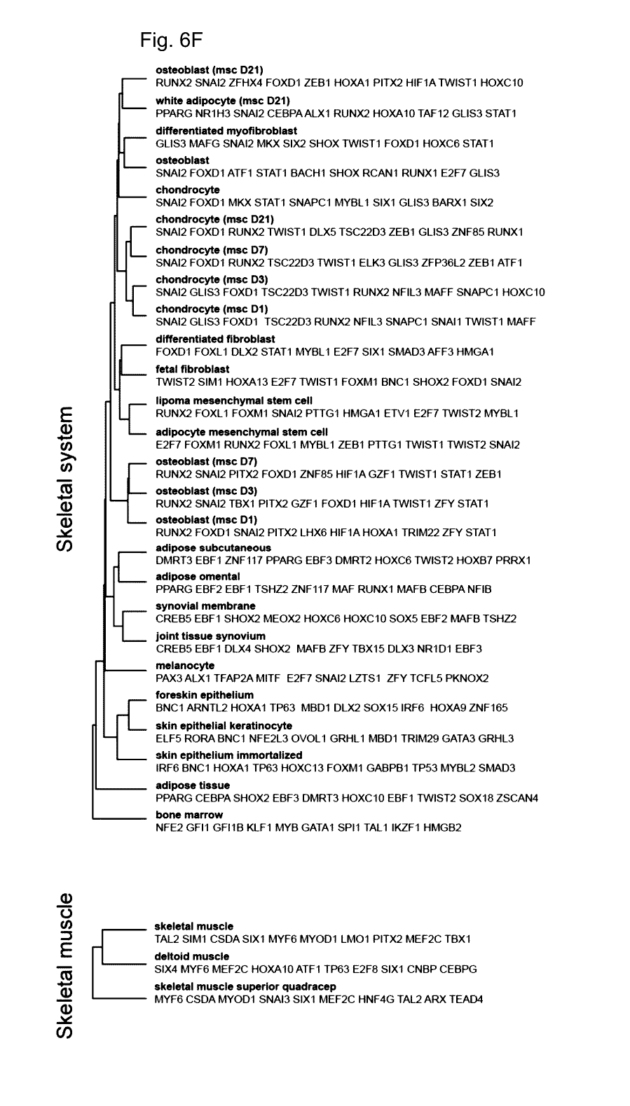

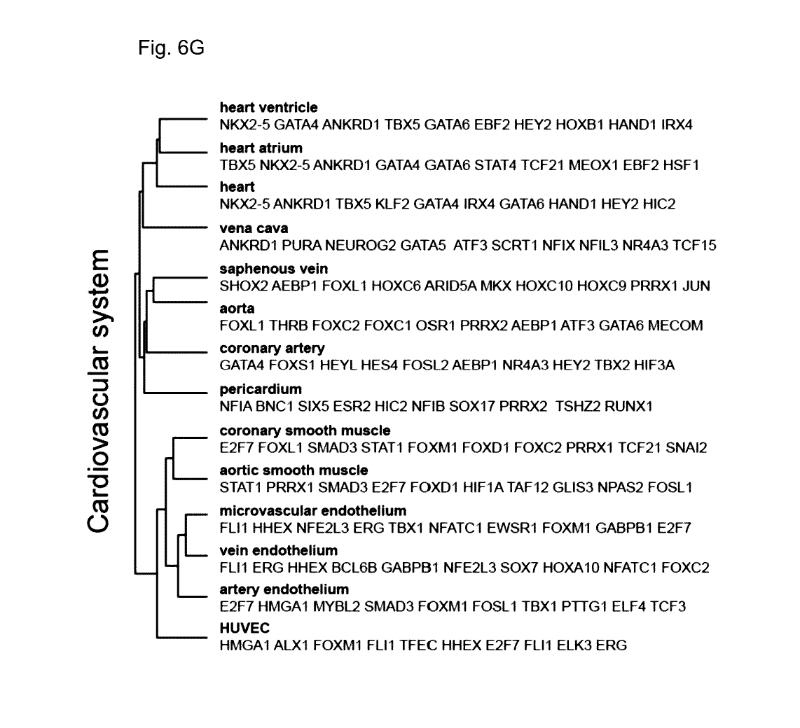

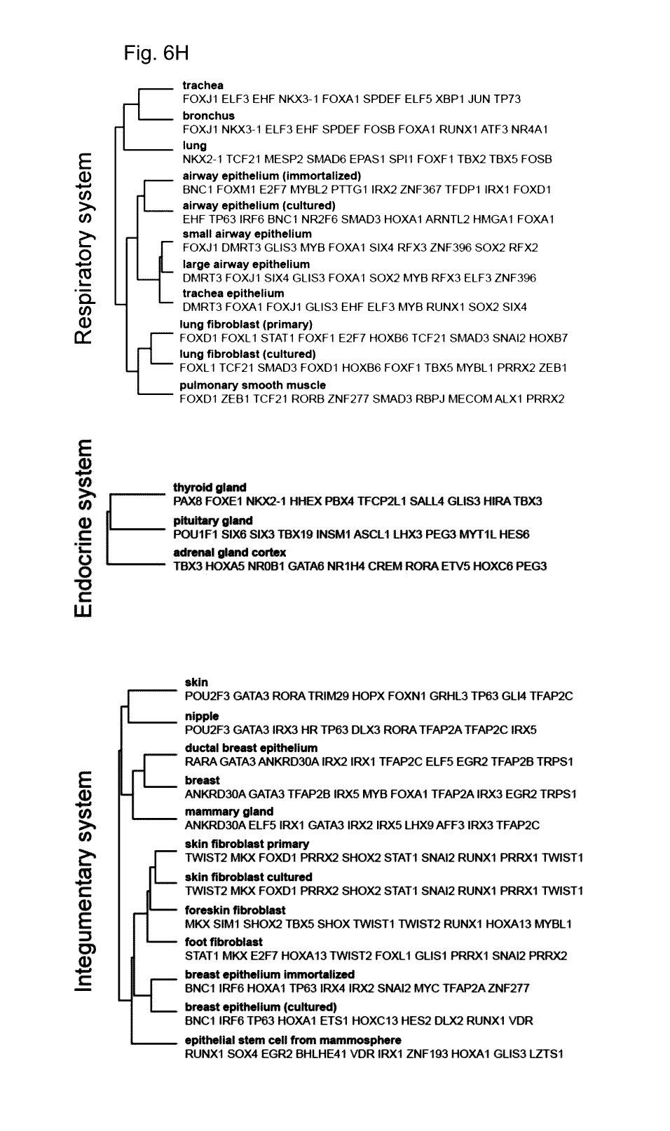

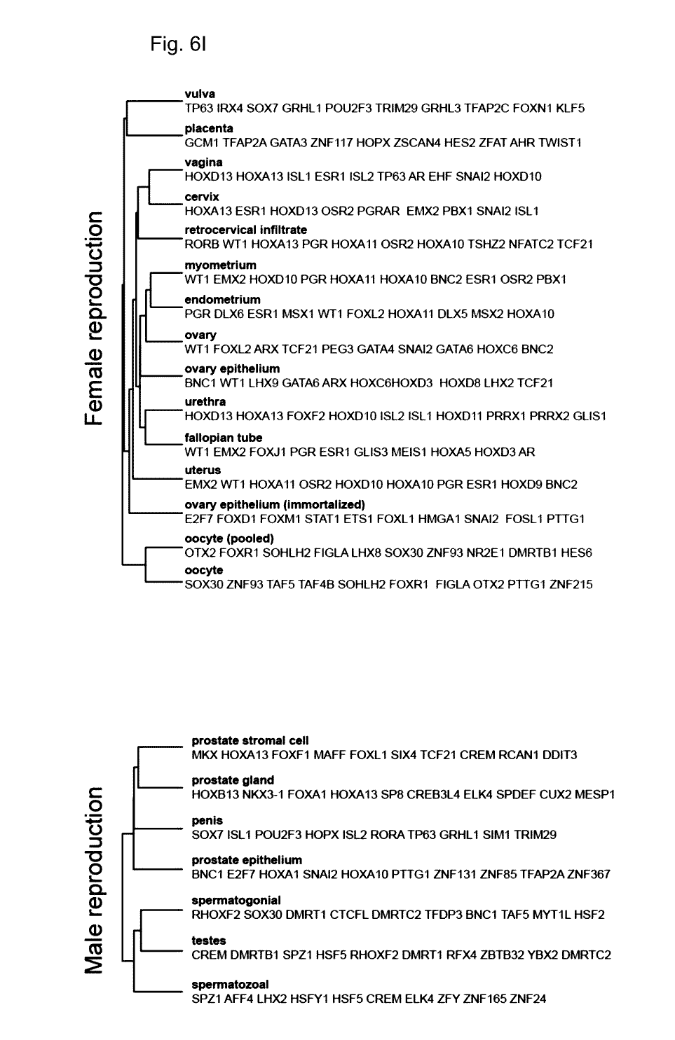

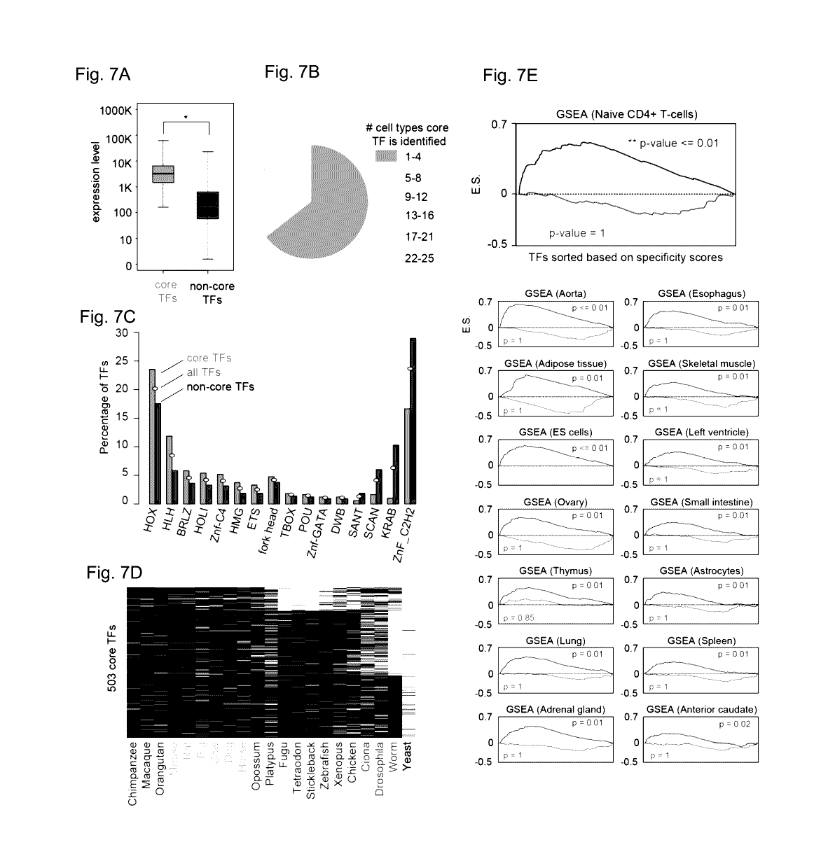

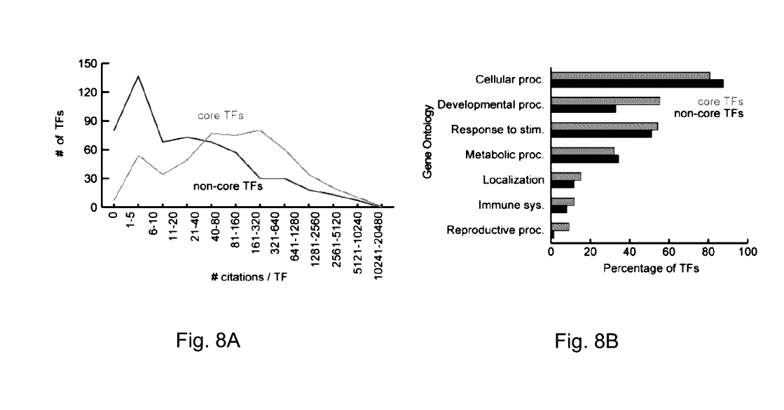

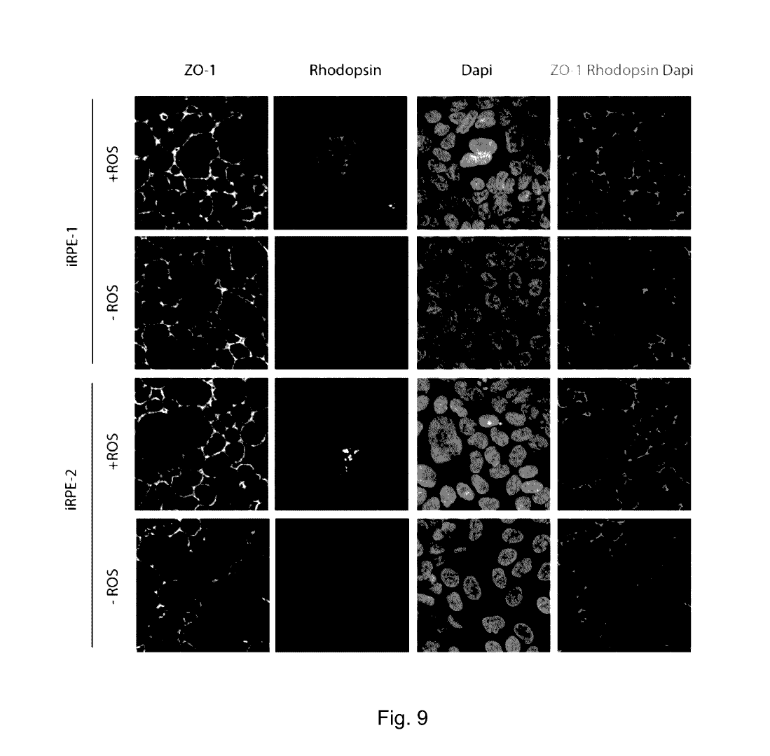

(FIG. 1A) Computational approach used to identify candidate master transcription factors in human cells. Left panel: Collection of gene expression profiles of a query cell type and representative cell types from Human Body Index collection of expression data. Middle panel: Expression profile of a single transcription factor across a query dataset and a range of background datasets. The idealized case of expression level of a transcription factor (grey circle, dashed line) is compared to the observed data to calculate the expression-specificity score of the transcription factor. Right panel: Plot depicting the distribution of significance scores of expression-specificity for all transcription factors. Factors are arranged on the x-axis in order of significance scores. Significance scores are indicated on the y-axis. The highest scoring transcription factors are considered the best candidate master transcription factors and highlighted in the red circle.

(FIG. 1B) Representation of the collection of candidate master transcription factors for 233 tissue and cell types. Tissue and cell types are arranged on the x-axis and clustered according to anatomical groups, represented by the colored bar at the top. Genes are arranged on the y-axis. Blue dashes represent candidate master transcription factors in a cell type. Clusters of candidate master transcription factors in cell types representing an anatomical group are boxed. Representative genes are listed on the side.

(FIG. 1C) List of top-scoring transcription factors in human ESCs ranked by expression specificity score. Asterisk indicates that the factor has been used in reprogramming experiments.

(FIG. 1D) List of top-scoring transcription factors in RPE cells ranked by expression specificity score.

FIGS. 2A-2F. Maintenance of RPE identity depends on candidate master transcription factors.

(FIG. 2A) qPCR validation of knockdown efficiency at 5 days post-infection with shRNA lentiviruses. The percent knockdown for two independent shRNA lentiviral constructs (1 and 2) for each candidate master transcription factor is shown. Results are normalized to a non-targeting shRNA control. All error bars reflect s.d. (n=2).

(FIG. 2B) RT-PCR expression analysis of the expression of transcripts of key RPE genes RPE65, TYR and CRALBP at 5 days post-infection with shRNA lentiviruses for candidate master TFs. Two independent shRNAs lentiviral constructs were used to knockdown each candidate master TF. Gene expression was normalized to GAPDH and calculated as a percent relative to non-targeting shRNA control±SD (n=2).

(FIG. 2C) Bar plot showing the number of differentially expressed genes that have absolute log 2-fold change≧1 relative to the non-targeting shRNA control following the knockdown of each of the eight candidate master TFs.

(FIG. 2D) Global gene expression analysis of RPE cells at 5 days post-infection with shRNA lentiviruses for candidate TFs. The heatmap indicates the fold change (log 2) of gene expression relative to the non-targeting shRNA control. Differentially expressed genes were combined and arranged in rows. The knockdown for each candidate master transcription factor or a non-targeting shRNA control are shown in columns. Knockdowns of candidate TFs cause reduced expression of key RPE genes including TIMP metallopeptidase inhibitor 3 (TIMP3), serpin peptidase inhibitor clade F member 1 (SERPINF1), transthyretin (TTR) and tyrosinase-related protein 1 (TYRP1) and increased expression of apoptotic genes including interferon-induced protein with tetratricopeptide repeats 2 (IFIT2), interferon, alpha-inducible protein 27 (IFI27) and phorbol-12-myristate-13-acetate-induced protein 1 (PMAIP1).

(FIG. 2E) GSEA of differentially expressed RPE genes at 5 days post-infection with shRNA lentiviruses. The differentially expressed genes after the knockdown of each candidate master transcription factor were combined and pre-ranked by the average fold changes across experiments relative to a non-targeting shRNA control. An RPE-signature gene set (n=152) from a previously published RPE transcriptome analysis (Strunnikova et al., 2010) was shown to be significantly down-regulated.

(FIG. 2F) Barplot showing the adjusted p-values (−log 10) of the top 10 enriched gene ontology terms for biological processes that are associated with the up-regulated genes after knockdown of candidate master transcription factors.

FIGS. 3A-3E. The transcriptional regulatory circuitry of human retinal pigment epithelial cells.

(FIG. 3A) Heat map showing the binding patterns for candidate master transcription factors at putative enhancer regions that show enrichment of H3K27AC (n=17,679). ChIP-seq read density is shown for a 5-kb span, centered on the putative enhancer regions. Color scale indicates ChIP-seq signal in units of rpm/bp.

(FIG. 3B) The overlap of the bound regions of PAX6, LHX2, OTX2, MITF, and ZNF92 with putative enhancer regions that show enrichment of H3K27AC (n=17,679). Bar plot depicts the number of putative enhancer regions that are bound by each transcription factor.

(FIG. 3C) Distribution of H3K27ac ChIP-seq signal across the 12,750 enhancer clusters. ˜17,500 active enhancers were stitched together into 12,750 enhancer clusters to identify super-enhancers (see Experimental Procedures). Increasing background-subtracted H3K27ac ChIP-seq signal was used to rank the enhancer clusters. 670 super-enhancers containing exceptionally high amounts of H3K27ac were identified. Sample genes associated with RPE biology and their respective super-enhancers are highlighted.

(FIG. 3D) Tracks showing ChIP-seq enrichment of the active enhancer mark H3K27Ac at selected gene loci together with the signal for PAX6, LHX2, OTX2, MITF, and ZNF92. ChIP-seq signals are shown on the y-axis in units of reads per million mapped reads per base pair (rpm/bp). The location and size of the super-enhancer is shown at the top of the tracks and gene models are shown at the bottom.

(FIG. 3E) A model for the core transcriptional regulatory circuitry of RPE cells. Interconnected loops are formed by PAX6, LHX2, OTX2, MITF, and ZNF92. Genes are represented by rectangles and proteins are represented by ovals.

FIGS. 4A-4F. Ectopic expression of RPE candidate master transcription factors is sufficient to drive the morphology and gene expression program of fibroblasts towards an RPE-like state.

(FIG. 4A) Schematic outlining the ectopic expression of candidate master transcription factors in human neonatal foreskin fibroblasts (HFF). Lentiviral constructs were induced to express candidate master transcription factors with doxycycline (Dox). Scale bar 50 um.

(FIG. 4B) PCR and gel analysis of transgene integration for iRPE lines. Positive control (DNA of the constructs used to generate lentivirus) and negative control reactions are shown. Six different iRPE lines, labeled 1-6 are shown. Genes are indicated on the side.

(FIG. 4C) Immunostaining of iRPE-1 and iRPE-2 cells. Cells were immunostained with TJP1 (ZO-1). Scale bar 50 um.

(FIG. 4D) Immunostaining imaging of RPE, iRPE-1 and iRPE-2 cells. Cells were immunostained for retinal pigment epithelial cells markers CRALBP (green) and RPE65 (red) and with DAPI (blue). Scale bar 50 um.

(FIG. 4E) Principle component analysis (PCA) comparing the gene expression profiles of iRPE cells to gene expression profiles of other cell types. Principal components are shown on the x-, y- and z-axes. The expression profiles of HFF (black), iRPE cells (blue), RPE (light green), iPS-RPE (green), iPS (red) and ES (orange red), 106 additional cell types (grey) are shown.

(FIG. 4F) GSEA enrichment score of a previously published RPE signature gene set (Strunnikova et al., 2010) compared with genes differentially expressed between iRPE and fibroblasts. Genes are ranked along the x-axis based on differential expression in iRPE cells versus fibroblasts, with more expressed in iRPE (red) to more expressed in fibroblasts (blue). Black tick marks indicate a gene from the RPE signature set. Enrichment score is shown on the y-axis. P-value for significance is shown.

FIGS. 5A-5D. RPE-like cells have functional characteristics.

(FIG. 5A) Schematic of the phagocytosis of photoreceptor outer segments (ROS) assay for iRPE function. Immunostaining for rhodopsin and DAPI are shown. The top panel of images shows immunostaining for rhodopsin. The lower panel of images shows the same fields with rhodopsin indicated in red and DAPI staining for DNA shown in blue. Scale bar 25 um.

(FIG. 5B) Schematic and results of trans-epithelial resistance (TER) assay for iRPE-1, iRPE-2 and hRPE (Salero et al., 2012). TER for fibroblasts (grey), hRPE cells (black), iRPE-1 (red) and iRPE-2 (gold) is 155.2±5 Ω·cm2, 211.4±4 Ω·cm2, 275.6±15 Ω·cm2, and 232.2±8 Ω·cm2, respectively. TER was assayed in at least 5 biological replicates and is displayed as mean+SD.

(FIG. 5C) Schematic and results for polarized release of VEGF assayed by enzyme-linked immunosorbent assay (ELISA). Values are shown for fibroblasts (grey), hRPE (black), iRPE-1 (red) and iRPE-2 (gold), with the apical secretion values as solid colors and the basolateral secretion values as striped colors. The ratio of VEGF release (basolateral/apical) is shown below each bar. N.D. non detectable. ELISA was assayed in biological duplicates and is displayed as mean+SD.

(FIG. 5D) Xenotransplant subretinal transplantations of wild-type albino Sprague-Dawley rats. Hematoxylin & eosin staining show pigmented donor cells iRPE-2 visible in the RPE layer. Single pigmented cells were identified in the host RPE layer in the doxycycline-treated group, but not in the control iRPE group that did not receive doxycycline (data not shown). Pigmented cells are indicated with <sign. Scale bar 50 um.

FIGS. 6A-6I. Candidate core transcription factors for 233 tissue and cell types. Tissue and cell types were grouped into categories corresponding to different anatomical systems in the human body. Within each category, tissue and cell types were ordered using hierarchical clustering. The distance matrix was calculated by first rank-ordering the specificity scores for all transcription factors in each tissue and cell type within a category and then finding the Kendall tau correlation coefficient for each pairwise comparison of tissue and cell types within the category. For each individual tissue or cell type, the top 10 scoring candidate core transcription factors are listed.

FIGS. 7A-7E. Characterization of candidate core transcription factors.

(FIG. 7A) Box plots depicting the expression levels of candidate core TFs and non-core TFs. The significance of the difference between two groups was determined using two-tailed Mann-Whitney test. For each plot, the top and bottom box edges mark the first and third quartiles while the solid black line within the box marks the median. The top whisker line marks the largest data point that is within 1.5 fold of the interquartile range from the third quartile. The bottom whisker line marks the smallest data point that is within 1.5 fold of the interquartile range from the first quartile. Candidate core TFs are shown in gold. Non core-TFs are shown in gray.

(FIG. 7B) Pie chart depicting the number of cell types in which a TF is considered as a candidate core TF.

(FIG. 7C) Bar chart representing the percentage of candidate core TFs and non-core TFs that are associated with different classes of DNA binding domains. The significance of the difference in distribution between candidate core TFs and non-core TFs across these categories is p<0.003 and was determined using Chi-square test. The gray oval indicates the percentage of all TFs that are associated with the class of DNA binding domains as a point of comparison. Abbreviations for protein domains are: HOX, homeodomain; HLH, helix-loop-helix; BRLZ, basic region leucine zipper; HOLI, ligand binding domain of hormone receptor; ZnF_C4, c4 zinc finger in nuclear hormone receptors; HMG, high mobility group; ETS, erythroblast transformation specific; FH, forkhead; TBOX, T-box; POU, Pit-Oct-Unc; ZnF_GATA, zinc finger binding to DNA consensus sequence [AT]GATA[AG]; DWB, domain B in dwarfin family proteins; SANT, SWI3-ADA2-N—CoR-TFIIB DNA-binding domain; SCAN, SCAN domain; KRAB, Krueppel associated box; ZnF_C2H2, zinc finger C2H2.

(FIG. 7D) Heatmap depicting the presence (blue) or absence (white) of orthologous genes in a species for each candidate core TF. The candidate core TFs are arranged as rows, species are shown as columns. Species labels are colored using the following scheme: blue (primate), orange (mammal), purple (vertebrates), green (metazoa) and black (eukaryote). In the image, rows are clustered according to k-mean clustering (n=3).

(FIG. 7E) GSEA enrichment plots depicting the relationship between super-enhancer associated genes and high expression-specificity scores. Top panel: GSEA plot for genes associated with super-enhancers in CD4+naïve T cells and expression-specificity score. Enrichment score is plotted on the y-axis. The x-axis represents genes ordered by specificity score. The relationship when ordered by the expression specificity scores from CD4+ naïve T cells is shown in blue. The relationship when ordered by the expression specificity scores from a non-matching cell type (ES cells) is shown in gray for comparison. P-values for each are shown. Subsequent panels show similar relationships in different cell types. For each panel, the cell type is indicated. Super-enhancer associated genes are from that cell type. Blue curves represent the relationship when ordered by expression specificity scores for that cell type. Gray curves represent the relationship when ordered by expression specificity scores for a non-matching cell type (ES cells). P-values for each are shown.

FIG. 8A. Line plot histogram of number of TFs and corresponding number of PubMed references. Bins representing ranges of PubMed references are shown on the x-axis. Number of TFs per bin are plotted on the y-axis. Candidate core TFs are shown in gold. Non-core TFs are shown in gray.

FIG. 8B. Bar chart depicting the percentage of core TFs and non-core TFs that are associated with annotations supported by experimental evidence from the Gene Ontology database. Percentage is plotted on the x-axis. Categories are labeled on the y-axis. Candidate core TFs are shown in gold. Non-core TFs are shown in gray.

FIG. 9. Immunostaining imaging of iRPE-1 and iRPE-2 for ZO-1, rhodopsin and DAPI are shown in the presence or absence of ROS. The panel of images shows the same fields with ZO-1, rhodopsin and DAPI staining for DNA. The merged image shows the same fields with ZO-1 indicated in green, rhodopsin indicated in red and DAPI staining for DNA shown in blue. Rhodopsin is not detected in the absence of ROS. Rhodopsin positive staining is detected in iRPE lines because the cells have phagocytosed the ROS.

### DETAILED DESCRIPTION

Hundreds of transcription factors (TFs) are expressed in each cell type, but cell identity can be induced through the activity of just a small number of core TFs. Systematic identification of these core TFs for a wide variety of cell types is currently lacking, and would establish a foundation for understanding the transcriptional control of cell identity in development, disease and cell-based therapy. Described herein is, among other things, a computational approach that generates an atlas of candidate core TFs for a broad spectrum of cells. The potential impact of the atlas was demonstrated, in one example, via cellular reprogramming efforts where candidate core TFs proved capable of converting fibroblasts to retinal pigment epithelial-like cells. These results suggest that candidate core TFs from the atlas can be a useful starting point for studying transcriptional control of cell identity and reprogramming in many cell types.

Methods and computer algorithms for identifying master transcription factors of a query cell type are provided herein. In one aspect, the method includes: providing gene expression data of a plurality of transcription factors for a query cell type; relatively quantifying expression level and expression specificity of each transcription factor in the query cell type against a background gene expression profile assembled from a collection of cell types by using an entropy-based measure of Jensen-Shannon divergence (JSD), thereby generating a cell-type-specificity score for each transcription factor; and ranking the plurality of transcription factors based on their corresponding cell-type-specificity scores, wherein top ranked transcription factors are identified as master transcription factors of the query cell type.

In some embodiments, the top 20, top 15, top 10, top 9, top 8, top 7, top 6, top 5, top 4, top 3, or more or less transcription factors, or any subset or combination thereof, are identified as master transcription factors of the query cell type of interest. The master transcription factors can be used to induce transdifferentiation of a somatic cell to the cell type of interest by, e.g., ectopically expressing the master transcription factors in the somatic cell. The resulting induced cell can be used in a cell or tissue replacement therapy. In some embodiments, autologous somatic cells obtained from a patient are subject to transdifferentiation, so that the resulting cells can be transplanted back to the same patient to minimize immune response that might otherwise be mounted against the cells and to avoid the potential need for immunosuppression.

In one example, master transcription factors of retinal pigment epithelium (RPE) cells have been identified using methods of the present disclosure. The top 10 ranked transcription factors include PAX6, LHX2, OTX2, SOX9, MITF, SIX3, ZNF92, GLIS3, C11orf9 and FOXD1. Ectopic expression of these master transcription factors, or a subset thereof in a somatic cell, e.g., fibroblast, can induce transdifferentiation into an RPE cell exhibiting characteristics of an endogenous RPE cell. Such induced RPE (iRPE) cells can be used in an RPE cell replacement therapy to treat ocular diseases such as age-related macular degeneration, macular edema (including diabetic macular edema), proliferative vitreoretinopathy, branch and central retinal vein occlusion, retinitis pigmentosa, retinal detachment, diabetic retinopathy, retinal degeneration, vascular retinopathy, uveitis, AIDS-related retinitis, choroidal and retinal neovascularization, or macular telangiectasia.

### DEFINITIONS

For convenience, certain terms employed herein, in the specification, examples and appended claims are collected here. Unless otherwise defined, all technical and scientific terms used herein have the same meaning as commonly understood by one of ordinary skill in the art to which this disclosure belongs.

The term “transdifferentiation” is used interchangeably herein with the phrase “reprogramming” and refers to the conversion of one differentiated somatic cell type into a different differentiated somatic cell type.

As used herein, the term “somatic cell” refers to any cells forming the body of an organism, as opposed to germline cells. In mammals, germline cells include the gametes (spermatozoa and ova) which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. Unless otherwise indicated the methods for direct conversion of a somatic cell, e.g., fibroblast to an iRPE cell can be performed both in vivo and in vitro (where in vivo is practiced when a somatic cell, e.g., fibroblast, is present within a subject, and where in vitro is practiced using isolated somatic cell, e.g., fibroblast, maintained in culture).

The term “retinal pigment epithelium” or “RPE” refers to the pigmented cell layer just outside the neurosensory retina that nourishes retinal visual cells, which is firmly attached to the underlying choroid and overlying retinal visual cells. The RPE has several functions, namely, light absorption, epithelial transport, spatial ion buffering, visual cycle, phagocytosis, secretion and immune modulation. Dysfunction of the RPE is found in diseases such as age-related macular degeneration (AMD), retinitis pigmentosa and diabetic retinopathy. Thus, iRPE cells can be used to treat these diseases by, e.g., transplantation or cell replacement therapy.

As used herein, the term “endogenous RPE cell” refers to an RPE cell in vivo or an RPE cell produced by differentiation of an embryonic stem cell into an RPE cell, and exhibiting an RPE cell phenotype. The phenotype of an RPE cell is well known by persons of ordinary skill in the art, and includes, for example, colonies having a cobblestone sheet morphology, gene expression signature (e.g., ZO-1, CRALBP and RPE65), phagocytosis of photoreceptor rod outer segments, formation of a barrier for ion transport, and polarized growth factor secretion.

The term “induced retinal pigment epithelium cell” or “iRPE cell” as used herein refers to an RPE or RPE-like cell having one or more RPE characteristics (e.g., morphology, gene expression, and function) produced by direct conversion from a somatic cell, e.g., a fibroblast.

The term “master transcription factors” or “master TFs” (used interchangeably with “core transcription factors” or “core TFs”) refer to those transcription factors that are important for the establishment and/or maintenance of cell state, and are expressed at high levels in specific cell types. Master TFs for RPE cells include, for example, one or more of PAX6, LHX2, OTX2, SOX9, MITF, SIX3, ZNF92, GLIS3, C11orf9 and/or FOXD1. In one example, PAX6, OTX2, MITF, SIX3, GLIS3 and FOXD1 are master TFs sufficient for establishment and/or maintenance of RPE cell state. In another example, PAX6, OTX2, MITF and SIX3 are master TFs sufficient for establishment and/or maintenance of RPE cell state.

The term “cell-type-specificity score” refers to an integrated score that represents the expression specificity and expression level of a transcription factor in a cell type of interest, relative to those of that transcription factor across a collection of different cell types.

The term “gene expression data” refers to the amount of gene expression, measured by RNA transcripts or protein products, and includes without limitation gene expression profiling by microarray or sequencing, non-coding RNA profiling by microarray or sequencing, chromatin immunoprecipitation profiling by microarray or sequencing, genome methylation profiling by microarray or sequencing, genome variation profiling by array, single nucleotide polymorphism array, serial analysis of gene expression, and/or protein array.

“Human Body Index” refers to the transcriptional profiling of 667 human tissue samples, available at Gene Expression Omnibus (GEO) accession No. GSE7307.

“Jensen-Shannon divergence” is a statistic method of measuring the similarity between two probability distributions.

A “probability vector” is a vector with non-negative entries that add up to one. A “vector” in mathematics is a collection of elements.

The term “Affymetrix MAS5” refers to a statistical algorithm developed by Affymetrix, Inc. (Santa Clara, Calif.) which produces absolute and comparison analysis results for gene expression arrays.

The term “ectopic” refers to a substance present in a cell or organism other than its native or natural place and/or level. For example, the term “ectopic expression” refers to the expression of a gene in an abnormal or non-natural place (e.g., cell, tissue or organ), and/or at an abnormal (increased or decreased) level in an organism or in vitro culture.

The term “expression” refers to the cellular processes involved in producing RNA and proteins, including where applicable, but not limited to, for example, transcription, translation, folding, modification and processing. “Expression products” include RNA transcribed from a gene and polypeptides obtained by translation of mRNA transcribed from a gene.

As used herein, “PAX6”, “LHX2”, “OTX2”, “SOX9”, “MITF”, “SIX3”, “ZNF92”, “GLIS3”, “C11orf9”, and “FOXD1” refer to Genbank accession Nos.: NP_000271 (human), NP_004789 (human), NP_001257452 (human), NP_000337 (human), NP_000239 (human), NP_005404 (human), NP_001274461.1 (human), NP_001035878.1 (human), NP_001120864.1 (human) and NP_004463.1 (human), respectively. These terms also encompass species variants, homologues, allelic forms, mutant forms, and equivalents thereof, including conservative substitutions, additions, deletions therein not adversely affecting the structure or function. In addition to naturally-occurring allelic variants of the sequences that may exist in the population (“wild-type sequences”), it will be appreciated that, as is the case for virtually all proteins, a variety of changes can be introduced into the wild-type sequences without substantially altering the functional (biological) activity of the polypeptides. Such variants are included within the scope of the terms “PAX6”, “LHX2”, “OTX2”, “SOX9”, “MITF”, “SIX3”, “ZNF92”, “GLIS3”, “C11orf9”, and “FOXD1”.

The term a “variant” in referring to a polypeptide could be, e.g., a polypeptide at least 80%, 85%, 90%, 95%, 98%, or 99% identical to full length polypeptide. The variant could be a fragment of full length polypeptide, e.g., a fragment of at least 10 or at least 20 contiguous amino acids of the wild type version of the polypeptide. In some embodiments, a variant is a naturally occurring splice variant. The variant could be a polypeptide at least 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof having an activity of interest such as the ability to directly convert fibroblasts to iRPE cells. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein.

One of skill in the art will be aware of, or will readily be able to ascertain, whether a particular polypeptide variant, fragment, or derivative is functional using assays known in the art. For example, the ability of a variant of a PAX6, LHX2, OTX2, SOX9, MITF, SIX3, ZNF92, GLIS3, C11orf9, and/or FOXD1 polypeptides to convert a somatic cell, e.g., fibroblast to an iRPE can be assessed using the assays as disclose herein in the Examples. Other convenient assays include measuring the ability to activate transcription of a reporter construct containing a PAX6, LHX2, OTX2, SOX9, MITF, SIX3, ZNF92, GLIS3, C11orf9, and/or FOXD1 binding site operably linked to a nucleic acid sequence encoding a detectable marker such as luciferase. One assay involves determining whether the PAX6, LHX2, OTX2, SOX9, MITF, SIX3, ZNF92, GLIS3, C11orf9, and/or FOXD1 variant induces a somatic cell, e.g., fibroblast to become an iRPE cell or express markers of an RPE cell or exhibit functional characteristics of an RPE cell as disclosed herein. Determination of such expression of RPE markers can be determined using any suitable method, e.g., immunoblotting. Such assays may readily be adapted to identify or confirm activity of agents that directly convert a somatic cell, e.g., fibroblast to an iRPE cell. In certain embodiments of the disclosure a functional variant or fragment has at least 50%, 60%, 70%, 80%, 90%, 95% or more of the activity of the full length wild type polypeptide.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operably linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g. EPV and EBV vectors.

As used herein, the term “transcription factor” refers to a protein that binds to specific parts of DNA using DNA binding domains and is part of the system that controls the transfer (or transcription) of genetic information from DNA to RNA.

The terms “decreased”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “subject” and “individual” are used interchangeably herein, and refer to an animal, for example, a human from whom cells can be obtained and/or to whom treatment, including prophylactic treatment, with the cells as described herein, is provided. For treatment of those conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the term “treating” refer to providing medical or surgical attention, care, or management to an individual. The individual is usually ill or injured, or at increased risk of becoming ill relative to an average member of the population and in need of such attention, care, or management. In some embodiments, the term “treating” and “treatment” refers to administering to a subject an effective amount of a composition, e.g., a composition comprising iRPE cell or their differentiated progeny so that the subject has a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, treatment can be “prophylactic” treatment, where the subject is administered a composition as disclosed herein (e.g., a population of iRPE cell or their progeny) to a subject at risk of developing an ocular disease as disclosed herein. In some embodiments, treatment is “effective” if the progression of a disease is reduced or halted. Those in need of treatment include those already diagnosed with an ocular disease or disorder, e.g., AMD, as well as those likely to develop an ocular disease or disorder due to genetic susceptibility or other factors such as family history, exposure to susceptibility factors, weight, age, diet and health.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

As used herein, the term “about” means within 20%, more preferably within 10% and most preferably within 5%.

It is understood that the detailed description and the examples provided herein are illustrative only and are not to be taken as limitations upon the scope of the disclosure. Various changes and modifications to the disclosed embodiments, which will be apparent to those of skill in the art, may be made without departing from the spirit and scope of the present disclosure. Further, all patents, patent applications, and publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### Identification of Candidate Master Transcription Factors

Cell identity is controlled in large part by the action of transcription factors (TFs) that recognize and bind specific sequences in the genome and regulate gene expression. While approximately half of all transcription factors are expressed in any one cell type (Vaquerizas et al., 2009), a small number of core TFs are thought to be sufficient to establish control of the gene expression programs that define cell identity (Buganim et al., 2013; Graf and Enver, 2009; Morris and Daley, 2013; Sancho-Martinez et al., 2012; Vierbuchen and Wernig, 2012; Yamanaka, 2012). It would be valuable to identify these core transcription factors for all cell types; an atlas of candidate core regulators would complement ENCODEs encyclopedia of regulatory DNA elements (Rivera and Ren, 2013; Stergachis et al., 2013), guide exploration of the principles of transcriptional regulatory networks, enable more systematic research into the mechanistic and global functions of these key regulators of cell identity, and facilitate advances in direct reprogramming for clinically relevant cell types (Henriques et al., 2013; Iwafuchi-Doi and Zaret, 2014; Soufi et al., 2012; Xie and Ren, 2013).

Core transcription factors that control individual cell identity have been identified previously, but systematic efforts to do so for most cell types have been relatively rare until recently. Early efforts focused on experimental identification of genes that were differentially expressed in one cell type compared to a small range of other cell types and shown to have roles in controlling specific cell identities. Examples include MyoD1, which can convert fibroblasts to muscle cells upon overexpression in fibroblasts (Tapscott et al., 1988) and Oct4, whose loss results in loss of the pluripotent cell population in the mammalian embryo (Nichols et al., 1998). More recently, cellular reprogramming experiments, where ectopic expression of transcription factors converts cells from one type to another, arose as a particularly stringent test of the ability of transcription factors to establish cell identity (Buganim et al., 2013; Graf and Enver, 2009; Morris and Daley, 2013; Sancho-Martinez et al., 2012; Vierbuchen and Wernig, 2012; Yamanaka, 2012). While powerful demonstrations of the role of transcription factors in control of cell identity, these experimental approaches are necessarily focused on specific cell types.

The development of genome-scale technologies has enabled more global attempts to predict candidate factors that control cell identity. Genome-wide gene expression and epigenome analysis across multiple cell types have been used to identify candidate core factors via computational methods (Cahan et al., 2014; Heinaniemi et al., 2013; Lang et al., 2014; Morris et al., 2014; Roost et al., 2015). While broad in scope, these studies assess their predictions using more easily scalable methods and typically do not assess whether predicted factors are sufficient to establish cell identity.

In one aspect, describe herein is the identification of candidate core TFs across the largest collection of different human cell types to date. A computational approach was devised to systematically identify candidate core transcription factors for most known human cell types. Importantly, it is demonstrated herein with ectopic expression experiments that these predictions can identify factors capable of converting cell identity, thus providing a stringent criterion that is not tested with other approaches to identify key transcription factors. For example, expression of core factors identified for retinal pigment epithelial (RPE) cells was sufficient to reprogram human fibroblasts into RPE-like cells. These cells were functionally characterized for their similarity to RPE cells derived from healthy individuals, and shown to share many features, including morphology, gene expression, ability to perform canonical RPE processes and to integrate to the host RPE layer in transplantation experiments. These results suggest that the atlas of candidate core transcription factors should be useful for reprogramming additional clinically important cell types and for systematically discovering the regulatory circuitries for these cells.

The control of gene expression programs is apparently dominated by a small number of master transcription factors, but these have yet to be identified for most cell human types (Buganim et al., 2013; Morris and Daley, 2013; Sancho-Martinez et al., 2012; Vierbuchen and Wernig, 2012; Yamanaka, 2012). To identify candidate master TFs for the large population of human cell types, a computational approach was devised to examine the relative levels and cell-type-specificity of transcription factor expression in a large population of different cell types. With this method, a list of candidate master transcription factors was obtained for each of more than 200 cell types (Table 1, Table 2). This computational method is modular and scalable and thus can be adapted to predict master TFs for additional cell types for which expression data is not yet available.

As shown in FIG. 1A, a computational approach was developed herein that exploits the feature that the master TFs that are known to be important for establishment or maintenance of cell state, and that are components of most successful reprogramming factor cocktails, are expressed at high levels in specific cell types (Lee and Young, 2013), to identify candidate master TFs in all cell types for which gene expression data is available. The algorithm quantifies both the relative level and the cell type specificity of gene expression by using an entropy-based measure of Jensen-Shannon divergence (Cabili et al., 2011; Fuglede, 2004) to compare the expression of a transcription factor in a cell type of interest (the query dataset) to the expression of that factor across a range of cell types and tissues (the background dataset). The algorithm assumes an idealized case where a transcription factor is expressed to a high level in a single cell type and not expressed in any other cell type, then generates a specificity score based on how well the actual data matches with this idealized case, and ranks each transcription factor using a nonparametric rank-product approach to aggregate the results from multiple query datasets for a given cell type (Breitling et al., 2004). This approach has additional features that make it flexible yet robust. It is modular and expandable to the expression profiles of disparate cell types from different laboratories. Multiple expression profiles of a query cell type can be used to increase the robustness of the predictions. The algorithm also takes advantage of the multiplicity of expression profiles to favor those gene probes that are ranked highly and consistently across multiple profiles.

Specifically, an entropy-based measure of Jensen-Shannon divergence (JSD) was adopted to evaluate the relative expression levels and expression specificity of transcription factors. The method quantified the expression level of a transcript in a query cell type relative to the expression patterns of the transcript across a background dataset of diverse human cell and tissue types. The major steps included collection of a background dataset, expression profile normalization, balancing of the background dataset, application of the JSD method, and integration of multiple datasets to generate a final ranking of transcription factors.

For the background dataset, in one example, 504 expression datasets, representing 106 cell and tissues types, were gathered primarily from the Human Body Index collection of expression datasets (Gene Expression Omnibus, GSE7307) (Guo et al., 2013; Zhang et al., 2011); the Human Body Index collection represents one of the largest and best curated repositories of expression datasets for human cell and tissue types. For additional cell and tissue types used as query datasets, publicly available expression datasets were used (Table 9). Other expression datasets can also be used in accordance with the normalization and balancing methods described herein.

All expression profiles used in this analysis were processed and normalized together to generate Affymetrix MAS5-normalized probe set values. CEL files were processed using the standard MAS5 normalization technique found in the affy package for the software program, R. The signals for multiple individual probes assigned to a transcript were aggregated into a single probeset value using the standard probe assignment method (“hgu133plus2cdf”).

The representation of cell and tissue types in the background dataset was balanced to evenly represent the diversity of expression patterns of transcription factors. If expression profiles from replicate samples or highly similar cell types are over-represented in the background expression dataset, the transcription factors that are highly specific to these cell types would be mistakenly considered as expressed in many different cell types. To construct a balanced background dataset, all profiles in the original background dataset were first clustered by similarity. Clusters of highly similar expression profiles were then identified, a single representative profile was chosen as the representative of the cluster, and other profiles in highly similar clusters were removed from the background dataset. For clustering, pair-wise comparisons were first performed on all expression profiles using Pearson correlation coefficients (PCCs). Hierarchal clustering then partitioned expression profiles into clusters based on the distance matrix derived from the PCCs. To choose a cutoff for partitioning expression profiles into clusters comprising highly similar expression profiles, the distribution of PCCs of expression profiles in the background dataset was empirically examined. The PCCs showed a bimodal distribution, suggesting there were two subpopulations of expression profiles, with the profiles of one group being more similar to each other. Examination of the profiles in the group with high PCCs indicated that many of the profiles were from redundant samples. This observation suggested that a cutoff separating the two subpopulations would be generally useful for removing redundant profiles from the background dataset. This bimodal distribution was fitted with a mixture model with two Gaussian distributions to identify a cutoff value and a PCC of 0.9 was chosen to best separate the two subpopulations in the bimodal distribution. This cutoff was applied to identify clusters of similar profiles. Once clusters of similar profiles were identified, the medoid of a cluster was selected as the representative profile for that cluster of similar profiles. The expression profiles in the final, balanced background dataset are shown in Table 9.

Jensen-Shannon divergence (JSD), as described in (Fuglede, 2004), was used to quantify the similarity between the observed pattern of transcription factor expression across cell types and the idealized pattern of a cell-type-specific master transcription factor across cell types. For each probeset that is mapped to a transcription factor, we created two same-sized, discrete probability vectors to represent the observed pattern and the ideal pattern. For the observed pattern, the vector was formed by values from the expression profiles of the query cell type and the balanced background dataset. The elements in this vector are divided by the sum so that the new normalized vector sums to 1. For the idealized pattern, the vector was formed by a value of 1 at the position equivalent to that of the query cell type and zeroes at all other positions. The distance metric between these two vectors was calculated using JSD and referred to as the cell-type-specificity score for the probeset. With this approach, the level of expression and the specificity of expression are incorporated into a single score, thus transcription factors scoring highly in either metric may score highly overall.

Where possible, multiple query datasets for a cell type of interest were used to identify candidate master transcription factors. The use of multiple query datasets theoretically helps identify the most robust candidate factors and should compensate to some degree for experimental and technical variability in gene expression experiments. One potential drawback is that datasets from different sources may purport to represent the same cells but may differ greatly due to differences in how the cells were obtained, heterogeneity of different cell populations or variations in growth conditions. If the differences between datasets are extreme, the use of multiple datasets may effectively cancel out relevant information. To compensate for this potential drawback, query datasets of the same cell types were compared by pair-wise Pearson correlation and datasets were grouped using hierarchical clustering. These subclusters can then be analyzed in a modular fashion, providing additional flexibility at this stage. Subclusters of datasets can be evaluated for suitability in inclusion, based on technical concerns. Subclusters of datasets may also reveal nuances of the underlying biology that may be instructive. For instance, subclusters that seem to represent different developmental stages of the same cell type may be separated at this stage, allowing for the selection of different sets of factors, biased by developmental stage. For this work, subclusters consisting of datasets that were largely dissimilar to other datasets (Pearson correlation coefficients less than 0.7 compared to other datasets) were removed from further consideration as we wished to provide a baseline set of candidate master transcription factors derived from the most representative, publicly available data.

To integrate information from multiple query datasets to yield a single ranking for a given cell or tissue type, rank product-based scores were next calculated for each probeset (Breitling et al., 2004). Only those query datasets that were retained after clustering as described above were included. Rank product-based scores tend to favor probesets that were ranked highly across multiple arrays and penalized probesets that scored highly in one or a few expression profiles. The main advantage of this rank product-based approach was that it favored consistency and did not require a “hard” cut-off when combining different datasets. The final ranked lists of candidate transcription factors are provided in Table 1. For additional characterization, candidate core factors are considered the set of factors that appear as a top 10 scoring transcription factor in any one cell type.

The identification of master TFs is significant since for the vast majority of human cell types, the master transcription factors and the transcriptional programs they control is poorly understood. Much of disease-associated sequence variation occurs in transcriptional regulatory regions (Farh et al., 2014; Hnisz et al., 2013; Maurano et al., 2012), but the transcriptional mechanisms that lead to disease pathology are understood in only a few instances. The approach described herein may facilitate more systematic identification of key transcription factors, mapping of regulatory circuitries and deducing underlying disease mechanisms.

### Candidate Master Transcription Factors

The above approach was used to predict master TFs for over 200 cell types/tissues collated from the Human Body Index collection of expression data together with some additional well-studied cell types (FIGS. 1B and 6A-6I, Table 1, Table 2). The complete atlas contains the scores for all transcription factors in all cell types, but for simplicity of additional analyses and manageability of experimental validation, the 10 top-scoring TFs in each cell type were focused on as the primary candidate core transcription factors.

503 different TFs were considered candidate core TFs for one or more cell types or tissues. As expected given our methodology, the candidate core TFs were expressed at higher levels than non-core TFs (FIG. 7A) and individual factors were generally considered candidate core TFs in limited numbers of cell or tissue types (FIG. 7B). DNA binding domain analysis indicates that the candidate core TFs have a different distribution of DNA binding domains compared to other TFs, with relatively increased frequencies of domains frequently associated with developmental regulators (homeobox and helix-loop-helix) and relatively decreased frequencies of other domains such as SCAN, KRAB and C2H2 zinc finger (FIG. 7C). The candidate core TFs are generally well conserved, as orthologues typically exist for the factors through vertebrate and metazoan species (FIG. 7D). The candidate core TFs are generally associated with super-enhancers, transcriptional regulatory elements that are associated with genes that play important roles in cell identity (Hnisz et al., 2013; Parker et al., 2013; Whyte et al., 2013). Gene set enrichment analysis shows that super-enhancers of each cell type are enriched among the highest scoring TFs in the atlas, and this enrichment occurs in a cell type-specific manner (FIG. 7E). These data are consistent with the highest scoring TFs in the atlas having roles in control of cell identity. More extensive characterization and discussion of the set of candidate core transcription factors is included in Supplemental Information and FIGS. 8A-8B.

Specifically, the 233 cell types or tissues studied are listed in the first row in Table 1, along with the top 20 transcription factors for each cell type/tissue. Table 1A shows a list of 1055 transcription factors.

TABLE 1A
Global list of transcription factors

AATF
ADNP
ADNP2
AEBP1
AFF1
AFF3
AFF3 ///
MLL
AFF4
AHCTF1
AHR
AIRE
AKNA
ALS2CR8
ALX1
ALX3
ALX4
ANKRD1
ANKRD30A
AR
ARID3A
ARID4A
ARID5A
ARID5B
ARNT
ARNT2
ARNTL
ARNTL2
ARX
ASCL1
ASCL2
ASCL3
ATF1
ATF2
ATF3
ATF4
ATF5
ATF6
ATF6B
ATF6B ///
TNXB
ATF7
ATOH1
ATOH7
BACH1
BACH2
BARHL1
BARX1
BARX2
BATF
BATF2
BATF3
BCL11A
BCL3
BCL6
BCL6B
BCLAF1
BHLHE22
BHLHE40
BHLHE41
BLZF1
BNC1
BNC2
C11orf9
CBFA2T2
CBFA2T3
CBFB
CBL
CC2D1A
CDX1
CDX2
CDX4
CEBPA
CEBPB
CEBPD
CEBPE
CEBPG
CEBPZ
CIR1
CIZ1
CLOCK
CNBP
CNOT7
CNOT8
CREB1
CREB3
CREB3L1
CREB3L2
CREB3L3
CREB3L4
CREB5
CREBL2
CREBZF
CREM
CRX
CSDA
CSNK2A1
///
NFE2L2
CSRNP1
CSRNP2
CSRNP3
CTCF
CTCFL
CTCFL
///
HMGB1
CTNNB1
CUX1
CUX2
DACH1
DBP
DDIT3
DEAF1
DLX1
DLX2
DLX3
DLX4
DLX5
DLX6
DMBX1
DMRT1
DMRT2
DMRT3
DMRTA1
DMRTB1
DMRTC2
DMTF1
DRAP1
DUX4 /// DUX4L2
/// DUX4L3 ///
DUX4L4 ///
DUX4L5 ///
DUX4L6 ///
DUX4L7
E2F1
E2F2
E2F3
E2F4
E2F5
E2F6
E2F7
E2F8
E4F1
EBF1
EBF2
EBF3
EBF4
EGR1
EGR2
EGR3
EGR4
EHF
ELF1
ELF2
ELF3
ELF4
ELF5
ELK1
ELK3
ELK4
EMX1
EMX2
EN1
EN2
EOMES
EP300
EPAS1
ERF
ERG
ESR1
ESR2
ESRRA
ESRRB
ESRRG
ESX1
ETS1
ETS2
ETV1
ETV2
ETV3
ETV4
ETV5
ETV6
ETV7
EVX1
EWSR1
/// FLI1
FAM36A
///
HOXA7
FBXO16
///
ZNF395
FERD3L
FEV
FEZF2
FIGLA
FLI1
FOS
FOSB
FOSL1
FOSL2
FOXA1
FOXA2
FOXA3
FOXB1
FOXC1
FOXC2
FOXD1
FOXD2
FOXD3
FOXD4
FOXD4
///
FOXD4L1
FOXE1
FOXE3
FOXF1
FOXF2
FOXG1
FOXH1
FOXI1
FOXJ1
FOXJ2
FOXJ3
FOXK1
FOXK2
FOXL1
FOXL2
FOXM1
FOXN1
FOXN2
FOXN3
FOXN4
FOXO1
FOXO3
FOXO3
///
FOXO3B
FOXO4
FOXP1
FOXP2
FOXP3
FOXP4
FOXQ1
FOXR1
FOXR2
FOXS1
FUBP1
FUBP3
GABPA
GABPB1
GAS7
GATA1
GATA2
GATA3
GATA4
GATA5
GATA6
GATAD1
GATAD2A
GATAD2B
GBX1
GBX2
GCFC1
GCM1
GCM2
GFI1
GFI1B
GLI1
GLI2
GLI3
GLI4
GLI4 ///
ZFP41
GLIS1
GLIS2
GLIS3
GMEB1
GMEB2
GPBP1
GRHL1
GRHL2
GRHL3
GSC
GSC2
GSX1
GSX2
GZF1
HAND1
HAND2
HBP1
HCFC1
HCLS1
HDX
HES1
HES2
HES4
HES5
HES6
HES7
HESX1
HEY1
HEY2
HEYL
HHEX
HIC1
HIC2
HIF1A
HIF3A
HINFP
HIRA
HIVEP1
HIVEP2
HIVEP3
HLF
HLX
HMBOX1
HMG20A
HMG20B
HMGA1
HMGB1
HMGB2
HMX1
HMX2
HNF1A
HNF1B
HNF4A
HNF4G
HOMEZ
HOPX
HOXA1
HOXA10
HOXA10
///
HOXA9
HOXA11
HOXA13
HOXA2
HOXA3
HOXA4
HOXA5
HOXA6
HOXA7
HOXB1
HOXB13
HOXB2
HOXB3
HOXB4
HOXB5
HOXB6
HOXB7
HOXB8
HOXB9
HOXC10
HOXC11
HOXC12
HOXC13
HOXC4
HOXC5
HOXC6
HOXC8
HOXC9
HOXD1
HOXD10
HOXD11
HOXD12
HOXD13
HOXD3
HOXD3 ///
HOXD4 ///
MIR10B
HOXD4
HOXD8
HOXD9
HR
HSF1
HSF2
HSF4
HSF5
HSFX1 ///
HSFX2
HSFY1 ///
HSFY2
ID1
ID3
IKZF1
IKZF2
IKZF3
IKZF4
IKZF5
ILF2
ILF3
INSM1
IRF1
IRF2
IRF3
IRF4
IRF5
IRF6
IRF7
IRF8
IRF9
IRX1
IRX2
IRX3
IRX4
IRX5
ISL1
ISL2
ISX
JDP2
JUN
JUNB
JUND
KLF1
KLF10
KLF11
KLF12
KLF13
KLF14
KLF15
KLF16
KLF17
KLF2
KLF3
KLF4
KLF5
KLF6
KLF7
KLF8
KLF9
L3MBTL1
L3MBTL4
LBX1
LCOR
LCORL
LEF1
LHX1
LHX2
LHX3
LHX4
LHX5
LHX6
LHX8
LHX9
LIN54
LMO1
LMO4
LMX1A
LMX1B
LOC100287728
/// ZNF75D
LOC253842 ///
NR6A1
LOC645895 ///
MYBL1
LOC729991-
MEF2B ///
MEF2B
LRRFIP1
LZTR1
LZTS1
MAF
MAFB
MAFF
MAFG
MAFK
MAX
MAZ
MBD1
MECOM
MECP2
MEF2A
MEF2C
MEF2D
MEIS1
MEIS2
MEIS3
MEOX1
MEOX2
MESP1
MESP2
MGA
MITF
MIXL1
MKX
MLLT10
MLLT3
MLX
MLXIP
MLXIPL
MNT
MNX1
MSC
MSX1
MSX2
MTA1
MTA2
MTA3
MTF1
MTF2
MXD1
MXD3
MXI1
MYB
MYBL1
MYBL2
MYC
MYCL1
MYCN
MYEF2
MYF5
MYF6
MYNN
MYOD1
MYOG
MYPOP
MYT1
MYT1L
MZF1
NANOG
NCOR1
NEUROD1
NEUROD2
NEUROD4
NEUROD6
NEUROG1
NEUROG2
NEUROG3
NFAT5
NFATC1
NFATC2
NFATC3
NFATC4
NFE2
NFE2L1
NFE2L2
NFE2L3
NFIA
NFIB
NFIC
NFIL3
NFIX
NFKB1
NFKB2
NFRKB
NFX1
NFXL1
NFYA
NFYB
NFYC
NHLH1
NHLH2
NKRF
NKX2-1
NKX2-2
NKX2-3
NKX2-5
NKX2-8
NKX3-1
NKX3-2
NKX6-1
NKX6-2
NKX6-3
NME1-
NME2 ///
NME2
NOBOX
NOTCH1
NPAS1
NPAS2
NPAS3
NPAS4
NROB1
NR0B2
NR1D1 ///
THRA
NR1D2
NR1H2
NR1H3
NR1H4
NR1I2
NR1I3
NR2C1
NR2C2
NR2E1
NR2E1
NR2F2
NR2F6
NR3C1
NR3C2
NR4A1
NR4A2
NR4A3
NR5A1
NR5A2
NR6A1
NRF1
NRL
OLIG1
OLIG2
OLIG3
ONECUT1
ONECUT2
ONECUT3
OSR1
OSR2
OTP
OTX1
OTX2
OVOL1
OVOL2
PA2G4
PA2G4 ///
PA2G4P4
PATZ1
PAX1
PAX2
PAX3
PAX4
PAX5
PAX6
PAX7
PAX8
PAX9
PBX1
PBX2
PBX3
PBX4
PCGF2
PCGF6
PDX1
PEG3
PGBD1
PGR
PHOX2A
PHOX2B
PHTF1
PITX1
PITX2
PITX3
PKNOX1
PKNOX2
PLAG1
PLAGL1
PLAGL2
POU1F1
POU2F1
POU2F2
POU2F3
POU3F1
POU3F2
POU3F3
POU3F4
POU4F1
POU4F2
POU4F3
POU5F1 ///
POU5F1B
///
POU5F1P3
///
POU5F1P4
POU5F1B
POU5F2
POU6F1
POU6F2
PPARA
PPARD
PPARG
PRDM1
PRDM14
PRDM2
PRDM5
PREB
PROP1
PROX1
PROX2
PRRX1
PRRX2
PTTG1
PURA
PURB
RARA
RARB
RARG
RAX
RAX2
RB1
RBPJ
RBPJL
RCAN1
RCOR2
REL
RELA
RELB
RERE
REST
RFX1
RFX2
RFX3
RFX4
RFX5
RFX6
RFX7
RFXANK
RFXAP
RHOXF1
RHOXF2
///
RHOXF2B
RNF141
RNF4
RORA
RORB
RORC
RREB1
RUNX1
RUNX1 ///
SH3D19
RUNX1T1
RUNX2
RUNX3
RXRA
RXRB
RXRG
SALL1
SALL2
SALL3
SALL4
SATB1
SATB2
SCRT1
SEBOX ///
VTN
SHOX
SHOX2
SIM1
SIM2
SIX1
SIX2
SIX3
SIX4
SIX5
SIX6
SLC2A4RG
SLC30A9
SMAD1
SMAD2
SMAD3
SMAD4
SMAD5
SMAD6
SMAD7
SMAD9
SNAI1
SNAI2
SNAI3
SNAPC1
SNAPC2
SNAPC3
SNAPC4
SNAPC5
SOHLH1
SOHLH2
SOLH
SOX1
SOX10
SOX11
SOX12
SOX13
SOX14
SOX15
SOX17
SOX18
SOX2
SOX21
SOX3
SOX30
SOX4
SOX5
SOX6
SOX7
SOX8
SOX9
SP1
SP140
SP2
SP3
SP4
SP5
SP6
SP7
SP8
SPDEF
SPEN
SPI1
SPIB
SPIC
SPZ1
SREBF1
SREBF2
SRF
SRY
ST18
STAT1
STAT2
STAT3
STAT4
STAT5A
STAT5B
STAT6
T
TADA2A
TAF10
TAF12
TAF13
TAF1B
TAF4
TAF4B
TAF5
TAF5L
TAF6
TAF7
TAL1
TAL2
TARDBP
TBP
TBPL1
TBR1
TBX1
TBX10
TBX15
TBX18
TBX19
TBX2
TBX20
TBX21
TBX22
TBX3
TBX4
TBX5
TBX6
TCF12
TCF15
TCF19
TCF20
TCF21
TCF25
TCF3
TCF4
TCF7
TCF7L1
TCF7L2
TCFL5
TEAD1
TEAD2
TEAD3
TEAD4
TEF
TEAM
TFAP2A
TFAP2B
TFAP2C
TFAP2D
TFAP2E
TFAP4
TFCP2
TFCP2L1
TFDP1
TFDP2
TFDP3
TFE3
TFEB
TFEC
TGIF1
TGIF2
TGIF2LY
THAP1
THAP11
THRA
THRB
TLX1
TLX2
TLX3
TP53
TP63
TP73
TRERF1
TRIM22
TRIM25
TRIM28
TRIM29
TRPS1
TSC22D1
TSC22D2
TSC22D3
TSC22D4
TSHZ1
TSHZ2
TSHZ3
TUB
TULP4
TWIST1
TWIST2
UBN1
UBP1
USF1
USF2
VAX2
VDR
VENTX
VEZF1
VSX1
WT1
XBP1
YBX1
YBX1 ///
YBX1P2
YBX2
YY1
YY2
ZBED1
ZBTB16
ZBTB17
ZBTB2
ZBTB20
ZBTB25
ZBTB32
ZBTB33
ZBTB34
ZBTB38
ZBTB4
ZBTB48
ZBTB5
ZBTB7A
ZBTB7B
ZC3H8
ZEB1
ZEB2
ZFAT
ZFHX2
ZFHX3
ZFHX4
ZFP36L1
ZFP36L2
ZFP37
ZFP42
ZFP57
ZFP90
ZFX
ZFX ///
ZFY
ZFY
ZGLP1
ZGPAT
ZHX1
ZHX2
ZHX3
ZIC1
ZIC2
ZIC3
ZIC4
ZIC5
ZKSCAN1
ZKSCAN2
ZKSCAN3
ZKSCAN4
ZKSCAN5
ZNF10
ZNF117
ZNF131
ZNF132
ZNF133
ZNF134
ZNF135
ZNF138
ZNF140
ZNF143
ZNF146
ZNF148
ZNF154
ZNF155
ZNF157
ZNF160
ZNF165
ZNF167
ZNF169
ZNF174
ZNF175
ZNF18
ZNF189
ZNF19
ZNF192
ZNF193
ZNF197
ZNF202
ZNF207
ZNF213
ZNF215
ZNF217
ZNF219
ZNF22
ZNF221
///
ZNF230
ZNF224
ZNF23
ZNF230
ZNF232
ZNF236
ZNF238
ZNF239
ZNF24
ZNF256
ZNF260
ZNF263
ZNF267
ZNF268
ZNF274
ZNF277
ZNF281
ZNF282
ZNF287
ZNF3
ZNF300
ZNF323
ZNF333
ZNF33A
ZNF33B
ZNF35
ZNF350
ZNF354A
ZNF354C
ZNF367
ZNF37A
ZNF37A
///
ZNF37BP
ZNF382
ZNF384
ZNF394
ZNF395
ZNF396
ZNF397
ZNF397OS
ZNF398
ZNF41
ZNF410
ZNF418
ZNF423
ZNF434
ZNF438
ZNF444
ZNF445
ZNF446
ZNF449
ZNF45
ZNF483
ZNF492
ZNF496
ZNF498
ZNF500
ZNF536
ZNF569
ZNF593
ZNF606
ZNF628
ZNF639
ZNF643
ZNF652
ZNF667
ZNF692
ZNF70
ZNF71
ZNF75D
ZNF76
ZNF8
ZNF80
ZNF81
ZNF83
ZNF85
ZNF90
ZNF91
ZNF92
ZNF93
ZNFX1
ZSCAN1
ZSCAN10
ZSCAN12
ZSCAN16
ZSCAN18
ZSCAN2
ZSCAN20
ZSCAN21
ZSCAN22
ZSCAN23
ZSCAN29
ZSCAN30
ZSCAN4
ZSCAN5A
ZXDA
ZXDA ///
ZXDB
ZXDC

Because embryonic stem cells (ESCs) are among the best-characterized cells, ESCs represented a useful first test case for the approach. The top-ranked factors for embryonic stem cells included the reprogramming factors OCT4/POU5F1, SOX2, NANOG, SALL4 and MYCN and additional factors known to be important for ESCs (ZIC2, ZIC3, OTX2, ZSCAN10) (FIG. 1C) (Avilion et al., 2003; Boyer et al., 2005; Chambers et al., 2003; Ivanova et al., 2006; Kim et al., 2008; Wang et al., 2007a; Wang et al., 2007b).

The top ranked factors for other well-studied cell types included the transcription factors that have been shown to be capable of trans-differentiating fibroblasts into various other cell types (Table 2). Specifically, embryonic stem cells, neural precursor cells, cardiomyocytes, hepatocytes, motor neurons, pancreatic islet cells, melanocytes and RPE were studied and the top 10 scoring candidate master transcription factors for each cell type are shown. For reference, the ranks of other transcription factors, in addition to the top 10, that have been used in reprogramming experiments are also shown. Transcription factors that have been used in reprogramming experiments are shown in bold. Certain TFs previously used in reprogramming experiments fall into the top 10 list of candidate TFs identified herein. It should be noted that the fact that some TFs rank relatively low may be due to several reasons, such as imperfect dataset publicly available at the present time and used herein. It is also possible that previous reprogramming studies have yet to identify the most effective master TFs such as those discovered herein for the first time.

TABLE 2
Top scoring candidates for select tissues and
comparison with known reprogramming factors

| Embryonic stem cells | | Neural precursor cells | | Cardiomyocytes | |
|---|---|---|---|---|---|
| Rank | TF | Rank | TF | Rank | TF |
| 1 | SALL4 | 1 | OTX2 | 1 | ANKRD1 |
| 2 | OTX2 | 2 | SALL4 | 2 | NKX2-5 |
| 3 | ZIC3 | 3 | SIX3 | 3 | E2F8 |
| 4 | NANOG | 4 | LHX2 | 4 | TBX5 |
| 5 | ZSCAN10 | 5 | SP8 | 5 | MEF2A |
| 6 | POU5F1 | 6 | SOX11 | 6 | ZBF193 |
| 7 | MYCN | 7 | TCF3 | 7 | MSX2 |
| 8 | NR6A1 | 8 | PAX6 | 8 | SOX11 |
| 9 | ZIC2 | 9 | ZIC2 | 9 | GATA4 |
| 10 | SOX2 | 10 | NR6A1 | 10 | LRRFIP1 |
| 329 | MYC | 18 | FOXG1 | 183 | HAND2 |
| 890 | KLF4 | 26 | SOX2 | | |
| | | 162 | ZIC1 | | |
| | | 200 | REST | | |
| | | 351 | POU3F2 | | |
| | | 458 | HES1 | | |
| | | 551 | RFX4 | | |
| | | 594 | NEUROG2 | | |
| | | 701 | ASCL1 | | |
| | | 719 | PLAGL1 | | |
| | | 834 | MYC | | |
| | | 1028 | KLF4 | | |

| Hepatocytes | | Motor neurons | | Pancreatic islet cells | |
|---|---|---|---|---|---|
| Rank | TF | Rank | TF | Rank | TF |
| 1 | NR1H4 | 1 | MNX1 | 1 | RFX6 |
| 2 | NR1I2 | 2 | ESRRG | 2 | INSM1 |
| 3 | HNF4A | 3 | ISL2 | 3 | PAX6 |
| 4 | NR5A2 | 4 | HOXC6 | 4 | ISL1 |
| 5 | HNF4G | 5 | CREM | 5 | NEUROD1 |
| 6 | ATF5 | 6 | NHLH1 | 6 | GLIS3 |
| 7 | NR1I3 | 7 | ZNF92 | 7 | NR5A2 |
| 8 | HHEX | 8 | GZF1 | 8 | ZNF165 |
| 9 | PROX1 | 9 | GLIS3 | 9 | ARX |
| 10 | FOXA3 | 10 | ISL1 | 10 | MNX1 |
| 106 | CEBPA | 57 | POU3F2 | 14 | MAFB |
| 192 | GATA4 | 65 | MYT1L | 371 | PDX1 |
| | | 90 | ASCL1 | 543 | PAX4 |
| | | 249 | NEUROG2 | 755 | NEUROG3 |
| | | 850 | LHX3 | | |

| | |
|---|---|
| Melanocytes | Retinal pigment epithelial |

| Rank | TF | Rank | TF |
|---|---|---|---|
| 1 | PAX3 | 1 | OTX2 |
| 2 | ALX1 | 2 | SIX3 |
| 3 | TFAP2A | 3 | LHX2 |
| 4 | MITF | 4 | PAX6 |
| 5 | E2F7 | 5 | FOXD1 |
| 6 | SNAI2 | 6 | MITF |
| 7 | LZTS1 | 7 | C11orf9 |
| 8 | ZFY | 8 | ZNF92 |
| 9 | TCFL5 | 9 | GLIS3 |
| 10 | PKNOX2 | 10 | SOX9 |
| 28 | SOX10 | 44 | NRL |
| | | 88 | CRX |
| | | 182 | RAX |
| | | 276 | MYC |
| | | 993 | KLF4 |

Activin A or RA plus
later SHH treatment

Thus, the compendium of candidate master TFs shown in Tables 1 and 2 is a useful resource for future studies of transcriptional regulatory networks and for reprogramming cell state. In some embodiments, for each of the cell types or tissues listed in Tables 1 and 2, the corresponding top 10 master TFs, or any subset thereof or combination therein, can be expressed (e.g., ectopically) in a somatic cell to induce trandifferentiation of the somatic cell into the target cell type or tissue. Several populations of somatic cells can each be induced to transdifferentiate into a different cell type or tissue in the same container or vessel, such that together they form a target organ.

In one aspect, the atlas of candidate core transcription factors presented herein provides a powerful starting point for studies of transcriptional regulation of cell identity and in applications for therapeutic purposes. The atlas itself is easily expanded with additional genome-wide expression data, which is relatively easy to obtain compared to other data types, especially for cell types that may be available in limiting quantities. The approach is easy to implement and can be adapted to next generation sequencing data as sufficient numbers and variety of datasets become available and may thus be generally useful for a wide range of users. The approach presented here capitalizes on basic principles of the expression level of known core TFs: relatively high expression and relatively cell type specific expression. In some embodiments, one or more additional principles commonly associated with core TFs, such as autoregulation, binding in regulatory regions, or motif enrichment in regulatory regions may be integrated into a method or system described herein.

The iRPE cells described herein represent the results of a stringent test for whether our approach successfully identifies transcription factors that can control cell identity. The factors here differ from, but overlap with a set of factors previously used for RPE reprogramming (Zhang et al., 2014). Significantly, known oncogenic transcription factors, such as MYC, and signaling molecules such as activin A or retinoic acid together with SHH were components of previous factor cocktails but are not required here. The iRPE generated here were characterized for morphology, gene expression and functionality and found to be largely similar to RPE, and thus, these cells represent functionally characterized iRPE cells. These cells require continued expression of at least one of the transgenes, as withdrawal of doxycycline causes the cells to revert back towards a fibroblast morphology, similar to many other transdifferentiated cells (Buganim et al., 2012; Huang et al., 2011; Lujan et al., 2012; Sheng et al., 2012; Vierbuchen et al., 2010), indicating establishment of a fully self-sustaining RPE identity may require one or more additional factors or other modifications. For example, in some embodiments, one or more additional TFs from the ranked list in Table 1 or the list in Table 1A may be ectopically expressed. We predict that analysis of additional factors from our ranked list, as well as analysis of additional transdifferentiated and differentiated versions of RPE cells (Idelson et al., 2009; Kamao et al., 2014; Zhang et al., 2014), will prove useful in ultimately unraveling the complete transcriptional circuitry of RPE cells.

Multiple methods have been developed that can use high-throughput genomic data to identify factors critical for cell identity (Benayoun et al., 2014; Cahan et al., 2014; Davis and Eddy, 2013; Heinaniemi et al., 2013; Hwang et al., 2011; Lang et al., 2014; Morris et al., 2014; Roost et al., 2015; Zhou et al., 2011; Ziller et al., 2015). Many of these methods focus primarily on quantifying the differences between cell identities and less on the direct identification of factors controlling cell identity. Several of these approaches have experimentally verified that they are capable of identifying transcription factors important for cell identity, although none has demonstrated the factors can establish cell identity to the extent shown here, possibly due to the extreme technical difficulty of these types of reprogramming experiments. Our expectation is that results for different methods of identifying candidate core TFs will eventually be compared and used in complementary fashions to gain insight on which TFs are critical for different cell types and which characteristics best define core TFs.

For the vast majority of human cell types, the core transcription factors and the transcriptional programs they control is poorly understood. Furthermore, much of disease-associated sequence variation occurs in transcriptional regulatory regions (Farh et al., 2014; Hnisz et al., 2013; Maurano et al., 2012), but the transcriptional mechanisms that lead to disease pathology are understood in only a few instances. The atlas of candidate core TFs described herein can therefore facilitate future exploration of the functions of key regulators of cell identity, mapping of cellular regulatory circuitries and investigation of disease-associated mechanisms.

### Somatic Cells

While fibroblasts are generally used, essentially any primary somatic cell type can be substituted for a fibroblast with the methods described herein. Some non-limiting examples of primary cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. The cell can be a primary cell isolated from any somatic tissue including, but not limited to brain, liver, lung, gut, stomach, intestine, fat, muscle, uterus, skin, spleen, endocrine organ, bone, etc.

Where the cell is maintained under in vitro conditions, conventional tissue culture conditions and methods can be used, and are known to those of skill in the art. Isolation and culture methods for various cells are well within the abilities of one skilled in the art.

Further, the parental cell can be from any mammalian species, with non-limiting examples including a murine, bovine, simian, porcine, equine, ovine, or human cell. For clarity and simplicity, the description of the methods herein refers to fibroblasts as the parental cells, but it should be understood that all of the methods described herein can be readily applied to other primary parent cell types. In some embodiments, the somatic cell is derived from a human individual.

In some embodiments, the methods and compositions of the present disclosure can be practiced on somatic cells that are fully differentiated and/or restricted to giving rise only to cells of that particular type. The somatic cells can be either partially or terminally differentiated prior to direct conversion to iRPEs or other cell types of interest. In some embodiments, somatic cells which are trandifferentiated into iRPEs or other cell types of interest are fibroblast cells.

In certain embodiments, the somatic cells can be normal or healthy cells. The somatic cells can also be diseased cells. For example, a cancer cell may be subject to the methods described herein so as to identify master TFs that can control or otherwise contribute to the cancerous state of the cell. Reducing or inhibiting expression of one or more such master TFs can then be used to remove the cell out of the cancerous state into, e.g., a healthier state.

### Reprogramming (Transdifferentiation)

The process of altering the cell phenotype of a differentiated cell (i.e. a first cell), e.g., altering the phenotype of a somatic cell to a differentiated cell of a different phenotype (i.e. a second cell) is referred to as “reprogramming” or “transdifferentiation”. Stated another way, cells of one type can be converted to another type in a process by what is commonly referred to in the art as transdifferentiation, direct reprogramming, cellular reprogramming or lineage reprogramming. Is should be noted that the term “reprogramming” or “transdifferentiation” also includes, altering the phenotype or state of a cell without changing its cell type, e.g., from a diseased cell to a healthy cell of the same cell type.

It was examined whether factors from the above-described atlas could induce a new cell identity as a stringent test of whether the atlas successfully identifies transcription factors that control cell identity. Ectopic expression of core TFs in fibroblasts can reprogram gene expression and produce cells with functional states similar to those that normally express those TFs (Buganim et al., 2013; Graf, 2011; Morris and Daley, 2013; Vierbuchen and Wernig, 2012; Yamanaka, 2012). Examination of the list of candidate core transcription factors predicted for embryonic stem cells shows good overlap with factors already used to reprogram murine or human fibroblasts to pluripotent stem cells (Table 2). Similar results are seen for several other cell types, including cardiomyocytes and hepatocytes (Table 2), and comparison to a set of transcription factors that have been used for lineage reprogramming in human cells—summarized in (Xu et al., 2015)—shows that roughly 70% of these lineage reprogramming factors are called as candidate core transcription factors in the atlas (Table 13). To test factors from this atlas, RPE cells were chosen as the target cell type due to their growing relevance to cell therapy applications. Progressive degeneration of RPE cells is a major cause of age-related macular degeneration (AMD), and several clinical trials are currently assessing transplantation of RPE cells and stem cell-derived RPE cells as a treatment for ocular disorders (Cyranoski, 2013, 2014).

TABLE 13
Transcription factors identified in atlas and previously
used to reprogram cells to specific lineages

| Transcription Factor | Previously Used To Reprogram To |
|---|---|
| ASCL1 | neuron |
| ATF5 | hepatocytes |
| CEBPA | hepatocytes |
| ERG#ERG1 | endothelial cells |
| ESRRG | cardiomyocytes |
| FLI1 | endothelial cells |
| FOSB | haematopoietic multipotent progenitor cell |
| FOXA2 | neuron |
| FOXA3 | hepatocytes |
| GATA4 | cardiomyocytes |
| GFI1 | haematopoietic multipotent progenitor cell |
| HNF4A | hepatocytes |
| HOXA11 | nephron progenitors |
| ISL1 | neuron |
| LHX3 | neuron |
| MEF2C | cardiomyocytes |
| MESP1 | cardiomyocytes |
| MITF | melanocyte, retinal pigment epithelium-like cells |
| MNX1#HB9 | neuron |
| MYC | retinal pigment epithelium-like cells |
| MYT1L#MYTL1 | neuron |
| NEUROD1 | neuron |
| NEUROG2#NGN2 | neuron |
| ONECUT1#HNF6 | hepatocytes |
| OSR1 | nephron progenitors |
| OTX2 | retinal pigment epithelium-like cells |
| PAX3 | melanocytes |
| PAX6 | retinal pigment epithelium-like cells |
| POU3F2#BRN2 | neuron |
| POU5F1#OCT4 | haematopoietic progenitors |
| PROX1 | hepatocytes |
| RUNX1 | haematopoietic multipotent progenitor cell |
| SIX1 | nephron progenitors |
| SIX2 | nephron progenitors |
| SNAI2 | nephron progenitors |
| SOX10 | melanocytes, neural crest cells |
| SOX2 | neural stem cells, neuroblasts |
| SPI1 | haematopoietic multipotent progenitor cell |
| TBX5 | cardiomyocytes |

As disclosed herein, the present disclosure relates to compositions and methods for the direct conversion of a somatic cell, e.g., a fibroblast to a cell type of interest, such as those cell types and tissues listed in Tables 1 and 2. Master transcription factors of the cell type of interest can be identified using methods described herein. In certain embodiments, master TFs can be the top 10 scoring ones listed in Tables 1 and 2. In further embodiments, a subset of the top 10 scoring master TFs can be sufficient to induce transdifferentiation, which can be ascertained via routine experimentation known to one of ordinary skill in the art (e.g., ectopic expression of various combinations of the top 10 TFs).

By increasing expression level of certain master transcription factors in a somatic cell, transdifferentiation into the cell type of interest can be induced. Various methods for increasing expression level known in the art can be used, including without limitation, contacting the somatic cell with an agent which increases the expression of the master transcription factors, such as a nucleotide sequence (e.g., encoding one or more of the master transcription factors), a protein, an aptamer, a small molecule, a ribosome, a RNAi agent, a peptide-nucleic acid (PNA), or analogues or variants thereof. In some embodiments, ectopic expression of the master transcription factors in the somatic cell induces transdifferentiation into the cell type of interest. Ectopic expression can be achieved via introduction of a transgene of the transcription factor (carried by, e.g., a vector, e.g., a viral vector such as retrovirus, lentivirus, adenovirus, adeno-associated virus, and/or nanoparticles). Alternatively or additionally, endogenous gene expression can also be increased by modulating transcriptional machinery such as activating its corresponding promoters and/or enhancers (e.g., using an artificial transcription factor comprising an activation domain or by introducing an activating mutation), recruiting transcription factors and/or RNA polymerase to the promoter/enhancer region, de-activating silencers, decreasing or removing repressors, etc. In some embodiments, epigenetic modification of the chromatin structure can be used to enhance endogenous gene expression.

In some embodiments, nucleic acids encoding multiple master TFs (e.g., 2, 3, 4, or more) may be incorporated into a vector under control of separate promoters or under control of the same promoter. For example, a polycistronic vector in which nucleic acid sequences encoding the polypeptides are separated by 2A peptides or IRES sequences may be used. Those of ordinary skill in the art are aware of 2A peptides, IRES sequences, and their use to co-express multiple polypeptides in cells, where the multiple polypeptides are encoded by a single mRNA. See, e.g., US Patent Application Pub. No. 20120028821 for further description of 2A peptides and their use to co-express multiple polypeptides in cells. In some embodiments, a transgene comprising a nucleic acid encoding the TF(s) may be integrated at a selected location such as a safe harbor locus (e.g., the adeno-associated virus integration site 1 (AAVS1) in human cells. In some embodiments, integration of a nucleic acid at a selected location in the genome may be achieved using genome editing systems such as CRISPR/Cas, TALENs, or zinc finger nucleases.

In some embodiments, ectopic expression of one or more master TFs may be achieved by introducing synthetic modified mRNA encoding the TF(s) into the cells. In some embodiments, synthetic modified mRNA comprises one or more nucleotides that are not normally found in naturally occurring mRNA encoding the master TFs. Such nucleotides may, for example, enhance stability and/or translation of the synthetic mRNA. Those of ordinary skill in the art are aware of suitable types of synthetic modified mRNA useful for expressing proteins in cells. See, e.g., US Patent Application Pub. No. 20120046346 and/or PCT/US2011/032679 (WO/2011/130624).

In certain embodiments, compositions and methods for transdifferentiation of a somatic cell, e.g., a fibroblast to a functional RPE cell, referred to herein as an “induced RPE (iRPE) cell” are provided. In certain embodiments, the transdifferentiation of a somatic cell, e.g., fibroblast causes the somatic cell to assume an RPE-like state. Transdifferentiation into iRPE cells can be achieved by increasing expression level of one or more of: PAX6, LHX2, OTX2, SOX9, MITF, SIX3, ZNF92, GLIS3, and FOXD1. In some embodiments, increased expression of at least two of, at least three of, at least four of, at least five of, at least six of, at least seven of, at least eight of, or all nine of PAX6, LHX2, OTX2, SOX9, MITF, SIX3, ZNF92, GLIS3, and FOXD1 induces transdifferentiation of somatic cells into iPRE cells. In one example, PAX6, OTX2, MITF, SIX3, GLIS3 and FOXD1 are master TFs sufficient for establishment and/or maintenance of RPE cell state. In another example, PAX6, OTX2, MITF and SIX3 are master TFs sufficient for establishment and/or maintenance of RPE cell state. In some embodiments the master TFs whose expression level is increased to establish and/or maintain an RPE cell state comprise OTX2, SIX3, GLIS3 and one, two, or more of PAX6, LHX2, SOX9, MITF, ZNF92, and FOXD1. For example, in some embodiments the master TFs comprise OTX2, SIX3, GLIS3, and FOXD1. In some embodiments the master TFs comprise OTX2, SIX3, GLIS3, and MITF. In some embodiments the master TFs comprise OTX2, SIX3, GLIS3, FOXD1, and MITF. In some embodiments the master TFs whose expression level is increased to establish and/or maintain an RPE cell state comprise do not include PAX6. In some embodiments the master TFs whose expression level is increased to establish and/or maintain an RIPE cell state comprise do not include MITF. In some embodiments the master TFs whose expression level is increased to establish and/or maintain an RPE cell state comprise do not include FOXD1.

Transdifferentiated cells have many clinical, therapeutic, and scientific applications. In some embodiments, the transdifferentiated cells can be transplanted to a patient in need of cell replacement therapy. The cells can be autologous to the patient, i.e., somatic cells from the patient can be first obtained, induced in vitro to transdifferentiate into one or more cell types of interest, and then transplanted back to the same patient. In one example, iRPE cells can be transplanted to treat age-related macular degeneration or other retinal dystrophies. In other embodiments, transdifferentiated cells can be cultured in vitro and/or subject to various in vitro experiments as a model for improving viability and/or to study their properties, and can be used to produce a substance (e.g., a protein) of interest or to generate artificial tissue/organ. In some embodiments, an artificial tissue or organ comprising one or more transdifferentatied cells may be introduced into a subject in need thereof, e.g., a subject in need of regeneration of the corresponding tissue or organ.

In some embodiments, cells that express one or more of the master TFs described herein may be used in methods (e.g., screening methods) to identify agents (e.g., small molecules (organic molecules having a molecular weight of 1.5 kilodaltons or less), nucleic acids (e.g., RNAi agents, microRNAs), or polypeptides) that may be used in a method of generating iRPE cells (or other cell types of interest described herein) to increase the efficiency of direct reprogramming and/or used instead of one or more of the master TFs described herein (as a substitute for one or more of the master TFs described herein) and/or to increase the efficiency of direct reprogramming. For example, in certain embodiments a population of somatic cells expressing one or more of the master TFs described herein is contacted with a test agent, and the ability of the test agent to increase the efficiency and/or speed of direct reprogramming to a cell type of interest is determined. Efficiency of direct reprogramming can be measured as the number of colonies of transdifferentiated cells of a cell type of interest (e.g., iRPE cells) that arise from a given number of somatic cells of a different cell type (e.g., fibroblasts) that have been modified to cause increased expression of one or more of the master TFs for the cell type of interest.

In some embodiments, iRPE cells (or other cells generated according to methods described herein) may be used as model systems, which may be used, e.g., for testing the potential efficacy and/or toxicity of agents such as candidate therapeutic agents or otherwise to evaluate the effect of agents or environmental conditions on the cells.

In some embodiments, iRPE cells (or other cells generated according to methods described herein) may be introduced into a non-human animal, e.g., a rodent or non-human primate, which non-human animal may be used as a model system. Such a model system may be used, e.g., for testing the potential efficacy and/or toxicity of agents such as candidate therapeutic agents or otherwise to evaluate the effect of agents or environmental conditions on the cells in vivo.

In some embodiments, cells that require continuous transgene expression to maintain their phenotype may be used for one or more applications, e.g., as model systems and/or in regenerative medicine. In some embodiments, the transgenes are expressed under the control of a promoter that is constitutively active in the starting cell type and in the transdifferentiated cell type. In some embodiments, the transgenes are expressed under the control of an inducible promoter. In some embodiments, an agent that induces expression of a transgene, such as doxycycline, is administered to a human or non-human mammal, into whom such cells are introduced, in order to maintain expression of the transgene in vivo. For example, in some embodiments an iRPE cell that requires continuous activation of transgene expression in order to maintain its phenotype may be introduced into the eye (e.g., beneath the retina). The recipient may be treated with doxycycline in order to maintain expression of the dox-inducible transgenes. To that end, in some embodiments an inducing agent such as doxycycline that is physiologically acceptable for administration, e.g., long-term administration (e.g., for at least 6 months), to a human or non-human mammal, may be used.

As an alternative to or, in addition to, ectopically expressing one or more master TFs, reducing or inhibiting the expression and/or activity of certain master TFs can also be desirable. For example, a cell in a first state may be determined to express one or more master TFs, and reducing or inhibiting the expression and/or activity of such master TFs can induce the cell to be out of the first state and/or enter a second state. The first state can be a diseased state (e.g., cancer) and the second state can be a healthy state (e.g., non-cancer). The first state can also be a differentiated state (e.g., differentiated immune cell such as memory B cell or memory T cell) and the second state can be a partially or completely de-differentiated state. In some embodiments the first state is an activated state and the second state is a non-activated state, or vice versa.

A variety of different agents and/or approaches may be used to inhibit expression of one or more master TFs. For example, in some embodiments RNA interference (RNAi) or an artificial TF may be used. In embodiments in which RNAi is used, the method may comprise introducing one or more RNAi agents, e.g., short interfering RNA (siRNA) or short hairpin RNA (shRNA), designed to inhibit expression of a master TF into the cell. In some embodiments one or more RNAi agent may be expressed intracellularly. Such expression may be constitutive or inducible in various embodiments. Those of ordinary skill in the art are aware of methods of designing and using RNAi agents to inhibit expression of a gene of interest. In some embodiments, a genome editing system such as CRISPR/Cas, TALEN, or zinc finger nuclease may be used to mutate a gene encoding a TF in order to reduce expression of the TFs in a cell or reduce the activity of the encoded protein. Mutations may be introduced into either or both alleles of the gene. A mutation may, for example, be introduced into a regulatory region such as a promoter or enhancer of the gene or into a coding region.

### Identification of RPE Master TFs and Use Thereof

The retinal pigment epithelium (RPE) provides vital support to photoreceptor cells and its dysfunction is associated with the onset and progression of age-related macular degeneration (AMD). Surgical provision of RPE cells may ameliorate AMD and thus it would be valuable to develop sources of patient-matched RPE cells for this application of regenerative medicine. Described herein is the generation of functional RPE-like cells from human fibroblasts that represent an important step toward that goal. Candidate master transcriptional regulators of RPE cells were identified using a computational method and then used to guide exploration of the transcriptional regulatory circuitry of RPE cells and to reprogram human fibroblasts into RPE-like cells. The RPE-like cells share key features with RPE cells derived from healthy individuals, including morphology, gene expression and function, and thus can be used to generate patient-matched RPE cells for treatment of macular degeneration or other ocular conditions.

Progressive degeneration of the retinal pigment epithelium is a major cause of age-related macular degeneration (AMD), which affects nearly 20% of individuals in aging populations (Lim et al., 2012). Surgical provision of healthy RPE cells has been used with some success in individuals with AMID (Binder et al., 2007; da Cruz et al., 2007) and there is considerable interest in generating patient-matched RPE cells for regenerative therapy. Human embryonic stem cell (ESC)-derived RPE cells have been transplanted into patients with AMD and initial results suggest visual improvement with no rejection or adverse outcomes (Schwartz et al., 2012; Schwartz et al., 2014). Several clinical trials are currently assessing the use of RPE cells in the treatment of ocular disorders (Cyranoski, 2013, 2014)(Clinical trials.gov NCT01674829, NCT01345006, NCT01344993, NCT01625559, NCT01469832). The RPE cells being used for these clinical trials are differentiated from human ESC or induced pluripotent stem cell (iPSC) lines (Kamao et al., 2014).

The potential of RPE cells for regenerative medicine has led to interest in the possibility that RPE cells might be obtained by direct reprogramming from fibroblasts, which is an alternative to the use of stem-cell-differentiated cells for cell-based replacement therapies. For some cell types, direct reprogramming can be achieved by ectopic expression of key transcription factors of the target cell type in cells of a different type (Buganim et al., 2013; Morris and Daley, 2013; Sancho-Martinez et al., 2012; Vierbuchen and Wernig, 2012; Yamanaka, 2012). Due to limited knowledge of the key factors for each cell type, referred to henceforth as master transcription factors, it is not currently possible to obtain various clinically relevant cell types by this approach. The identification of master transcription factors in all cell types might thus facilitate advances in direct reprogramming for clinically relevant cell types, including RPE cells.

Described herein is the identification of candidate master transcriptional factors of RPE cells and the use of these factors to investigate the transcriptional regulatory circuitry of RPE cells and to reprogram human fibroblasts into RPE-like cells. The computational approach described herein was used to systematically identify candidate master transcription factors for most known human cell types, including RPE cells. Genome-wide binding profiles of the predicted RPE master transcription factors generated a model of RPE core regulatory circuitry. Ectopic expression of predicted RPE master transcription factors in human fibroblasts produced cells that share key features with RPE cells derived from healthy individuals, including morphology, gene expression and function. These results suggest that the approach described here is useful for systematically identifying master transcription factors, discovering regulatory circuitries and reprogramming cells for additional clinically important cell types.

Certain of the methods described herein may be implemented at least in part using a computer. In some aspects, described herein is a non-transitory computer-readable medium storing computer-executable instructions for identifying master TFs of a cell type of interest. In some aspects, described herein is a non-transitory computer-readable medium storing computer-executable instructions for identifying master TFs of a cell type of interest. In some embodiments, described herein is a method that comprises causing the processor of a computer to execute instructions to identify master TFs of a cell type of interest as described herein. The instructions may be embodied in a computer program product comprising a computer-readable medium.

### RPE Master Transcription Factors, Super-Enhancers and Core Circuitry

To improve understanding of the transcriptional control of RPE cells, a study of the candidate master TFs identified for these cells was carried out (FIG. 1D). Nine top scoring transcription factors were selected—PAX6, LHX2, OTX2, SOX9, MITF, SIX3, ZNF92, GLIS3, and FOXD1—for further study. Among these, PAX6, OTX2 and MITF have previously been implicated in retinal pigment cell development (Bharti et al., 2012; Martinez-Morales et al., 2003; Matsuo et al., 1995), and SOX9 has been shown to interact with OTX2 and MITF (Martinez-Morales et al., 2003; Masuda and Esumi, 2010). Furthermore, PAX6, OTX2, MITF and five other TFs (MYC, KLF4, NRL, CRX and RAX) have been shown to induce an RPE-like progenitor state in fibroblasts (Zhang et al., 2014).

Well-studied master TFs are essential for maintenance of the gene expression program that controls cell identity, so we determined whether the RPE master TF candidates are essential for maintenance of the RPE gene expression program. We successfully knocked-down expression of eight (PAX6, OTX2, SOX9, MITF, SIX3, ZNF92, GLIS3 and FOXD1) of the nine candidate factors in human RPE cells (FIG. 2A, Table 3). Efficient knockdown of LHX2 was not successful, despite multiple attempts with several shRNA constructs. For each the eight TFs where efficient knockdown was achieved, reduced levels of the TF mRNA led to reduced expression of three well-studied genes known to be key to RPE function: RPE65, CRALBP and TYP (FIG. 2B). RPE65 and CRALBP encode two proteins that function in the visual cycle and TYR encodes an enzyme responsible for melanin biosynthesis in RPE melanosomes (Chiba, 2014; Fuhrmann et al., 2014; Sparrow et al., 2010; Strauss, 2005). Microarray analysis of gene expression revealed that the knockdown of the eight candidate master TFs had somewhat different quantitative effects (FIG. 2C), but there was a common set of 1700 differentially expressed genes (FDR of 0.01 with absolute log 2-fold change≧1) (FIG. 2D, Table 4), suggesting that RPE cells are similarly dependent on these factors for expression of this core set of genes. Examination of the down-regulated genes in this core set of genes showed significant enrichment of signature genes important for RPE function (FIGS. 2D and 2E). This RPE signature consisted of 154 highly expressed RPE genes previously identified by comparing the gene profiles of RPE cells to the Novartis expression database of 78 tissues (SymAtlas: wombat.gnf.org/index.html) (Strunnikova et al., 2010). In contrast, the up-regulated genes were associated with apoptotic cell death and cellular defense responses (FIGS. 2D and 2F). The morphological features of the cells were consistent with the induction of an apoptotic cell death program. These results indicate that the knockdown of the eight candidate master TFs caused a loss of the RPE cell expression program and subsequent induction of apoptosis. The similarity of the effects on gene expression observed with knockdown of these eight TFs suggests that they play similarly important roles in maintenance of the RPE gene expression program.

Studies of master TFs in embryonic stem cells and several differentiated cell types suggest that these factors share three common features (Lee and Young, 2013; Whyte et al., 2013). These factors bind enhancers for a substantial fraction of the genes that are actively transcribed, they bind clusters of enhancers (super-enhancers) at genes with prominent roles in cell-type specific biology, and they often bind the enhancers of their own genes as well as those of the other master TFs, thus forming a core circuitry of interconnected autoregulatory loops. To determine if the RPE candidate master TFs share these features, we identified RPE enhancers genome-wide and investigated the association of the RPE TFs with these enhancers (FIG. 3A). Active enhancers were identified by using chromatin immunoprecipitation coupled to massively parallel sequencing (ChIP-Seq) with antibodies against the histone modification H3K27ac (Table 5), a nucleosomal modification that occurs at active enhancers (Creyghton et al., 2010; Rada-Iglesias et al., 2011). The results indicated that RPE cells have at least 17,679 sites with high confidence signal for histone H3K27ac (FIG. 3A). We then carried out ChIP-Seq for the candidate master TFs and were able to obtain good quality data for five of the TFs (PAX6, LHX2, OTX2, MITF and ZNF92) (FIG. 3A). The high confidence data revealed that these five candidate master TFs together occupied at least one third of the 17,500 active enhancers (FIG. 3B).

To determine whether the candidate master TFs bind super-enhancers at their own genes and those of other key cell identity genes, the ChIP-seq signal for H3K27ac was used to identify super-enhancers and their associated genes (FIG. 3C, Table 6). The ChIP-seq data for the TFs was used to ascertain the pattern of TF binding to these super-enhancers (FIG. 3D). The RPE super-enhancers occurred at many genes associated with RPE transcriptional control, including the candidate master transcription factors SIX3, LHX2, OTX2 and FOXD1, and genes that feature prominently in RPE biology, including the retinal reductase gene DHRS3 (FIG. 3C, Table 6). Examination of the super-enhancers revealed that different combinations of the five TFs occupied the various enhancer components of the super-enhancers (FIG. 3D), as has been observed for master TFs at ESC super-enhancers (Whyte et al., 2013).

We next investigated whether the five candidate master TFs bind enhancers associated with their own genes as well as those associated with the other master TFs. The genome-wide binding data revealed that PAX6, LHX2 and OTX2 occupy active enhancers of genes encoding all five factors studied here, while MITF and ZNF92 occupied a subset of these enhancers (FIG. 3E). Thus, the RPE master TF candidates form a core circuit with interconnected autoregulatory loops whose characteristics are similar to those previously described for other well-studied cells such as ESCs (Lee and Young, 2013), hepatocytes (Odom et al., 2006), hematopoietic stem cells and erythroid cells (Novershtern et al., 2011) and T cell acute lymphoblastic leukemia cells (Sanda et al., 2012). A map of extended regulatory circuitry can be constructed for RPEs that includes genes that are both co-bound by all these regulators and dependent on their expression (FIG. 3E; Table 7).

These results show that the RPE transcription factors studied here share key features with established master transcription factors, including binding to a large fraction of active enhancers, occupancy of super-enhancers at their own genes and those of other key cell identity genes, and formation of core circuitry with interconnected autoregulatory loops.

Reprogramming of Fibroblasts into RPE-Like Cells

Ectopic expression of master TFs can, for many cell types, reprogram gene expression programs and produce cells with functional states like those that normally express those master TFs (Buganim et al., 2013; Morris and Daley, 2013; Sancho-Martinez et al., 2012; Vierbuchen and Wernig, 2012;

Yamanaka, 2012). We therefore investigated whether the nine top scoring RPE master TF candidates can reprogram fibroblasts into an RPE-like state (FIG. 4). For ectopic expression experiments, nine of the top scoring RPE core TF candidates PAX6, LHX2, OTX2, SOX9, MITF, SIX3, ZNF92, GLIS3, and FOXD1 were selected and cloned into doxycycline inducible lentiviral expression vectors (FIG. 4A). Human foreskin fibroblasts (HFF) were then transduced with a cocktail of all nine factors. Colonies showing a “cobblestone”-like morphology characteristic of RPE cells were evident after two weeks of doxycycline induction. These colonies increased in size over two months in culture (FIG. 4A). Independent cobblestone RPE-like colonies were manually picked and further expanded into six independent RPE-like cell lines. All six cell lines were found to contain the PAX6, OTX2, MITF, SIX3, GLIS3 and FOXD1 expression constructs (FIG. 4B, Table 8).

Two of the induced RPE-like cell lines, iRPE-1 and iRPE-2, were subjected to additional analysis. The iRPE cell lines exhibited characteristic expression of membrane-associated TJP1 (ZO-1) together with a “cobblestone” sheet morphology involving individual cells connected by tight junctions (FIG. 4C), and maintained an RPE-like morphology in the presence of doxycycline for over 6 months (twelve passages). Additionally, immunostaining indicates the iRPE cells showed co-expression of CRALBP and RPE65 (FIG. 4D), two well-known markers for RPE cells (Sparrow et al., 2010). Expression analysis shows the candidate core transcription factors are expressed in both iRPE lines and genes considered part of the RPE gene expression signature (Strunnikova et al., 2010) show substantial upregulation compared to fibroblasts (Table 12). Principal component analysis (PCA) of genome-wide gene expression revealed the two iRPE lines were as similar to primary RPE and induced pluripotent stem cell-derived RPE as induced pluripotent stem cells are to embryonic stem cells (FIG. 4E). Analysis of the genes differentially expressed between iRPE and fibroblasts shows that the gene expression differences between iRPE cells and fibroblasts correlate with the gene expression signature found in normal RPE cells (FIG. 4F) (Strunnikova et al., 2010).

Ectopic expression of the RPE candidate core TFs results in cells that are functionally similar to RPE cells. RPE play crucial roles in the maintenance and function of retinal photoreceptors, including phagocytosis of shed outer segments of photoreceptors (Bok, 1993), transepithelial transport of nutrients and ions between the neural retina and the blood vessels (Strauss, 2005), and secretion of growth factors and hormones (Ford et al., 2011). For assaying phagocytosis, mouse rod outer segments (ROS) were incubated with iRPE cells or HFF cells. ROS incorporation was measured using an antibody against rhodopsin, which specifically recognizes a component of ROS. Both iRPE cell lines stained positive for rhodopsin, indicating binding and incorporation of ROS into the RPE cells by phagocytosis (FIG. 4A, FIG. 9). To measure ion transport barrier function, we analyzed transepithelial electrical resistance (TER), which detects functional tight junctions (Stevenson et al., 1986). iRPE cells demonstrated effective barrier function that was significantly higher than fibroblasts and was as effective as that observed for RPE cells (FIG. 5B). To evaluate secretion of growth factors, iRPE cells were examined for production of vascular endothelial growth factor (VEGF), which is released preferentially to the basolateral side of RPE cells to prevent endothelial cell apoptosis in the blood vessels (Saint-Geniez et al., 2009). No VEGF release was detected when fibroblasts were assayed (FIG. 5C). The iRPE lines exhibit polarized secretion of VEGF similar to that produced by RPE cells (FIG. 5C). Subretinal transplantations experiments showed that iRPE cells survive in vivo when transplanted in albino rats and some integrate to the host RPE layer as pigmented cells (FIG. 5D). Taken together, these results provide the most extensive characterization of iRPE to date and indicate that cells generated with the factors from our atlas are similar to RPE in terms of morphology, gene expression and functionality.

iRPE Function

RPE cells play crucial roles in the maintenance and function of retinal photoreceptors, including phagocytosis of shed outer segments of photoreceptors, transepithelial transport of nutrients and ions between the neural retina and the blood vessels, and secretion of growth factors and hormones. To test if iRPE cells can perform typical RPE functions, we cultured iRPE cells and RPE cells in transwells for 8 weeks to obtain RPE sheets. We then tested whether the iRPE cells were capable of phagocytosis of photoreceptor rod outer segments, able to form a barrier for ion transport, and capable of polarized hormone secretion (FIG. 5).

Phagocytosis of photoreceptor rod outer segments (ROS) by RPE is essential for retinal function (Bok, 1993). The essential role of RPE phagocytosis is highlighted by the rapid degeneration of photoreceptor neurons and subsequent blindness occurring in Royal College of Surgeons rats, which carry an autosomal recessive mutation that impairs RPE phagocytosis (Bok and Hall, 1971). To test if iRPE cells can perform phagocytosis, we incubated mouse ROS with iRPE cells or HFF cells and tested for ROS incorporation using an antibody to rhodopsin. Both iRPE cell lines stained positive for rhodopsin, indicating binding and incorporation of ROS into the RPE cells by phagocytosis (FIG. 5A).

The RPE has structural properties of an ion transporting epithelium that controls transport of ions and water from the subretinal space, or apical side, to the blood vessels or basolateral side (Strauss, 2005). Tight junctions between cells prevent ion and water movement between the apical and basolateral sides of the cells. We evaluated this barrier function by measuring the transepithelial electrical resistance (TER), which provides a method to detect functional tight junctions (Stevenson et al., 1986). iRPE and RPE cells were cultured in transwells for 8 weeks prior to TER measurements. The mean TER was 275.6±17 Ω·cm² and 232.2±10 Ω·cm² for iRPE 1-2 clones, respectively, and 211.4±5 Ω·cm², for RPE cells (FIG. 5B). Thus, the iRPE cells were able to form an effective a barrier for ion transport and this was as effective as that observed for RPE cells.

The RPE produces and secretes a variety of growth factors and hormones to the apical and basolateral sides to maintain the structural properties of the retinal and blood vessels respectively (Ford et al., 2011). Vascular endothelial growth factor (VEGF) is released to the basolateral side preferentially and functions to prevent endothelial cell apoptosis in the blood vessels (Saint-Geniez et al., 2009). We cultured iRPE cells and RPE cells (Salem et al., 2012) in transwells and analyzed VEGF concentration secreted into the media from both apical and basolateral sides using ELBA. VEGF levels were 2,150±190 and 2660±63 pg/ml for the apical and basolateral sides respectively for iRPE-1, 1,731±5 and 3050±226 pg/ml for the apical and basolateral side respectively for iRPE-2 and 3,835±190 and 5548±691 pg/ml for the apical and basolateral side respectively for RPE (FIG. 5C), indicating a polarized secretion of VEGF in the iRPE lines that is similar to that produced by RPE cells.

We conclude that the iRPE cell lines are capable of three functions established for RPE cells: phagocytosis of photoreceptor rod outer segments, formation of a barrier for ion transport, and polarized growth factor secretion.

### SUMMARY

The retinal pigment epithelium provides vital support to photoreceptor cells and its dysfunction is associated with the onset and progression of age-related macular degeneration and other retinal dystrophies. We undertook a study of the master transcription factors of RPE cells to improve our understanding of the control of RPE gene expression and to explore whether these factors might facilitate generation of functional RPE-like cells from fibroblasts. RPE candidate master transcriptional regulators were identified using the computational method described herein and these were used to guide exploration of the transcriptional regulatory circuitry of RPE cells, core features of which we describe here. The candidate master transcriptional regulators were also used to reprogram human fibroblasts into RPE-like cells (iRPEs). The iRPE cells share key features with RPEs derived from healthy individuals, including morphology, gene expression and functional attributes, and thus represent a step toward the goal of generating patient-matched RPE cells for treatment of macular degeneration.

The candidate master TFs for RPE cells were used to deduce key features the transcriptional regulatory circuitry of these cells. Knockdown experiments showed that these TFs play an important role in the expression of RPE signature genes identified previously (Strunnikova et al., 2010). These TFs occupied enhancers associated with a third of the actively transcribed RPE genes, bound super-enhancers at their own genes and those for additional genes with prominent roles in RPE cell identity, and formed a core regulatory circuitry with interconnected autoregulatory loops. These features are shared by master TFs of other well-studied cells (Hnisz et al., 2013; Lee and Young, 2013; Novershtern et al., 2011; Sanda et al., 2012).

The RPE candidate master transcriptional regulators were used to reprogram human fibroblasts into iRPE cells that share key features with RPEs derived from healthy individuals, including morphology, gene expression and functional attributes. The generation of iRPE cells is an important step toward the goal of more efficient generation of patient-matched RPE cells for treatment of macular degeneration and other retinal dystrophies. The generation of autologous transplantation strategies may have particular value for elderly patients, who are more susceptible to complications from the immunosuppressive treatments that often accompany other transplantation strategies. These iRPE cells require continuous activation of transgene expression to stably maintain their morphology over 6 months. Similar dependency on constitutive transgene activity has been observed for the transdifferentiated state in other cases (Buganim et al., 2012; Huang et al., 2011; Lujan et al., 2012; Sheng et al., 2012; Vierbuchen et al., 2010), and transgene-independent lines can further be developed for regenerative medicine applications. It is possible that other TFs that scored highly in the computational approach described herein will facilitate full transgene-independent reprogramming.

### Exemplary Experimental Procedures

### Identification of Candidate Master Transcription Factors

Briefly, an entropy-based measure of Jensen-Shannon divergence (Cabili et al., 2011) was adopted to identify candidate master transcription factors, based on the relative level and cell-type-specificity of expression of a given factor in one cell type compared to a background dataset of diverse human cell and tissue types. Expression datasets used are provided in Table 9.

### Cell Culture

Human retinal pigment epithelial (RPE) cells used for ChIP-seq and knockdown experiments were purchased from ScienCell (ScienCell, cat. #6540). RPE cells were maintained in epithelial cell medium (EpiCM) (ScienCell, cat. #4101) supplemented with 2% fetal bovine serum (ScienCell, cat. #0010), lx epithelial cell growth supplement (EpiCGS) (ScienCell, cat. #4152), and 1× penicillin/streptomycin solution (ScienCell, cat. #0503). Human foreskin fibroblasts (HFF) were purchased from GlobalStem (GlobalStem, cat. #GSC-3002) and maintained in DMEM (Life Technologies, cat. #11965-092) supplemented with 15% of Tet System Approved fetal bovine serum (Clontech, cat. #631101), 2 mM L-Glutamine (Life Technologies, cat. #25030-081) and 100 U/ml penicillin-streptomycin (Life Technologies, cat. #15140-163).

### Knockdown of Candidate Master Transcription Factors

shRNAmir lentiviral vectors were obtained from Thermo Scientific (Table 3). A non-targeting shRNAmir was used as a control. High-titer lentiviral particles for each plasmid were used to transduce RPE cells (ScienCell, cat. #6540). Twenty-four hours after infection, epithelial cell medium was replaced and selection with 1 μg/ml puromycin (Life Technologies, cat. #A1113803) was carried out. Puromycin-resistant cells were harvested for future analysis five days after transduction.

TABLE 3
shRNAmir used

| ShRNAmir | Clone ID |
|---|---|
| PAX6#1 | V2HLS_75684 |
| PAX6#2 | V3HLS_645966 |
| OTX2#1 | V2LHS_404868 |
| OTX2#2 | V2LHS_87162 |
| MITF#1 | V2LHS_257541 |
| MITF#2 | V3LHS_388761 |
| SIX3#1 | V3LHS_371547 |
| SIX3#2 | V3LHS_371548 |
| FOXD1#1 | V3LHS_404368 |
| FOXD1#2 | V3LHS_17123 |
| GLIS3#1 | V3LHS_400168 |
| GLIS3#2 | V3LHS_320306 |
| SOX9#1 | V3LHS_396212 |
| SOX9#2 | V2LHS_11387 |
| ZNF92#1 | V2LHS_191304 |
| ZNF92#2 | V3LHS_414307 |

RNA Extraction, cDNA Preparation and Gene Expression Analysis

Total RNA from cultured cells was isolated using the RNeasy Mini Kit (Qiagen, cat. #74104), and cDNA was generated with SuperScript III First-Strand Synthesis System (Life technology, cat. #18080-051), following the manufacturer's suggested protocol. Quantitative real-time qPCR were carried out on the Applied Biosystems 7300 Real-Time PCR System (Applied Biosystems) using gene-specific Taqman probes from Life Technologies (Table 10) and TaqMan Universal PCR Master Mix (Life Technologies, cat. #4364340), following the manufacturer's suggested protocol. For microarray analysis, total RNA was harvested and used for library preparation. For each transcription factor, total RNA was harvested from two different lines, each harboring a different shRNAmir construct. 100 ng of total RNA was used to prepare biotinylated cRNA (cRNA) using the 3′ IVT Express Kit (Affymetrix, cat. #901228), following the manufacturer's suggested protocol. GeneChip Primeview Human Gene Expression Arrays (Affymetrix, cat. #901837) were hybridized and scanned following the manufacturer's suggested protocols. Additional details are provided below.

TABLE 10
Taqman probes used in this study

| Gene | Catalogue Number |
|---|---|
| PAX6 | Hs00240871_m1 |
| LHX2 | Hs00180351_m1 |
| OTX2 | Hs00222238_m1 |
| MITF | Hs01117294_m1 |
| SIX3 | Hs00193667_m1 |
| ZNF92 | Hs00705767_s1 |
| FOXD1 | Hs00270117_s1 |
| SOX9 | Hs01001343_g1 |
| GLIS3 | Hs00541450_m1 |
| GAPDH | Hs02758991_g1 |
| RPE65 | Hs01071462_m1 |
| TYR | Hs00165976_m1 |
| CRALBP | Hs00165632_m1 |

### ChIP-Seq and Analysis

Chromatin immunoprecipitation coupled with massively parallel sequencing (ChIP-seq) was performed as previously described (Lee et al., 2006; Marson et al., 2008). Antibodies used for ChIP-seq are provided in Table 5.

ChIP protocols have previously been described in detail (Lee et al., 2006). RPE cells were grown to passage 4 and crosslinked by the addition of one-tenth volume of fresh 11% formaldehyde solution for 12 minutes at room temperature. Cells were rinsed twice with 1×PBS, pelleted by centrifugation and flash frozen in liquid nitrogen and stored at −80° C. Cell pellets were resuspended, lysed and sonicated to solubilize and shear crosslinked DNA. We used a Bioruptor (Diagenode) and sonicated at medium power for 10×30 second pulses (30 second pause between pulses). Samples were kept on ice at all times. The resulting input material was incubated overnight at 4° C. with 20 μl of Dynal Protein G magnetic beads (Life Technologies, cat. #10004D) that had been pre-incubated with 5 μg of the appropriate antibody. The immunoprecipitation was allowed to proceed overnight at 4° C. For MITF, OTX2, PAX6, ZNF92, LHX2 immunoprecipitations, beads were washed twice with 20 mM Tris-HCl pH8.0, 150 mM NaCl, 2 mM EDTA, 0.1% SDS, 1% Triton X-100, once with 20 mM Tris-HCl pH8.0, 500 mM NaCl, 2 mM EDTA, 0.1% SDS, 1% Triton X-100, once with 10 mM Tris-HCl pH8.0, 250 nM LiCl, 2 mM EDTA, 1% NP40 and once with TE containing 50 mM NaCl. For RNA Pol II and H3K27Ac immunoprecipitations, sodium deoxycholate (0.1% final concentration) was added to all washes except the final TE wash. Bound complexes were eluted from the beads by heating at 65° C. with occasional vortexing and crosslinking was reversed by incubation at 65° C. for eight hours. Input material DNA (reserved from the sonication step) was also treated for crosslink reversal. Immunoprecipitated DNA and input material DNA were then purified by treatment with RNAse A, proteinase K and phenol:chloroform:isoamyl alcohol extraction. The antibodies used for ChIP analysis are listed in Table 5.

TABLE 5
Antibodies Used

| Antibody | Source | Catalog Number | Host species | Application | Notes |
|---|---|---|---|---|---|
| H3K27AC | Abcam | ab4729 | Rabbit polyclonal | Chip-seq | 5 μg per IP with 10\hat{ }7 cells |
| MITF | Active Motif | 39789 | Mouse monoclonal | Chip-seq | 5 μg per IP with 10\hat{ }7 cells |
| PAX6 | Abcam | ab5790 | Rabbit polyclonal | Chip-seq | 5 μg per IP with 10\hat{ }7 cells |
| OTX2 | Abcam | ab21990 | Rabbit polyclonal | Chip-seq | 5 μg per IP with 10\hat{ }7 cells |
| LHX2 | Santa Cruz | sc-19342X | Goat polyclonal | Chip-seq | 5 μg per IP with 10\hat{ }7 cells |
| RNA PolII | Santa Cruz | SC-899X | Rabbit polyclonal | Chip-seq | 5 μg per IP with 10\hat{ }7 cells |
| ZNF92 | Abgent | AT4646a | Mouse monoclonal | Chip-seq | 5 μg per IP with 10\hat{ }7 cells |
| ZO1 | Invitrogen | 18-7430 | Rabbit polyclonal | Staining | used at a 1:100 dilution |
| RPE65 | Dr. T. Michael Redmond, | | Rabbit monoclonal | Staining | used at a 1:100 dilution |
| | NEI | | | | |
| CRALBP | Abcam | ab15051 | Mouse monoclonal | Staining | used at a 1:100 dilution |
| Rhodopsin (1D4) | Santa Cruz Biotechnology | sc-57432 | Mouse Monoclonal | Staining | used at a 1:200 dilution |
| Alexa Fluor ® 488 | Life Tecnologies | A11001 | Goat anti-mouse IgG | Staining | used at a 1:1000 dilution |
| Alexa Fluor ® 555 | Life Tecnologies | A21430 | Goat anti-rabbit IgG (H + L) | Staining | used at a 1:1000 dilution |
| F(ab')2 | | | | | |
| Alexa Fluor ® 594 | Life Tecnologies | A11032 | Goat Anti-Mouse IgG | Staining | used at a 1:1000 dilution |
| Alexa-Fluor 546 | Life Tecnologies | A11035 | Goat anti rabbit | Staining | used at a 1:1000 dilution |

All ChIP-Seq datasets were aligned to build version NCBI37/HG19 of the human genome using Bowtie (version 0.12.9) (Langmead et al., 2009) with the following parameters: -n2, -e70, -m2, -k2, -best. We used the MACS version 1.4.1 (Model based analysis of ChIP-Seq) (Zhang et al., 2008) peak finding algorithm to identify regions of ChIP-Seq enrichment over background. A p-value threshold of enrichment of 1e-7 was used for all datasets with parameter -no-model, -dup=2. Approximately 15,200, 13,700, 9,400, 3,300, 12,500, regions were identified for LHX2, OTX2, PAX6, MITF, ZNF92, respectively. Wiggle files for gene tracks were created using MACS with options -w-S-space=50 to count reads in 50 bp bins. They were normalized to the total number (in millions) of mapped reads producing the final tracks in units of reads per million mapped reads per by (rpm/bp).

### Construction of Lentivirus-Inducible Vectors and Ectopic Expression Experiments

The Lenti-X Tet-On Advanced Inducible Expression System (Clontech, cat. #632162) was used for ectopic expression experiments. For construction of lentiviral vectors, the inducible vector backbone (pLVX-Tight-Puro) was first modified to include an MluI site in the linker region for potential future cloning steps. Next, plasmids containing the full coding sequence of PAX6, OTX2, LHX2, MITF, SIX3, SOX9, GLIS3, FOXD1, or ZNF92 were obtained from Open Biosystems, Origene or the Dana Farber/Harvard Cancer Center DNA Resource Core (Table 11). Coding DNA sequences were amplified using oligos that also added small regions of DNA homologous to regions flanking the MluI site in the target vector (Table 11). Target vector was then cut with MluI and the amplified coding DNA sequences were cloned into the target vector via homologous recombination using the In-Fusion cloning system (Clontech, cat#639646). Expression plasmids were transformed and maintained in STBL4 cells (Life Technologies, cat#11635-018).

TABLE 11
Primers and cDNA used for construction of lentiviral vectors

| | cDNA Information | | Primers for Amplification and Cloning | |
|---|---|---|---|---|
| | | Catalog | | |
| Factor | Source | number | Forward primer | Reverse primer |
| PAX6 | Open | MHS1010- | cccaggtcccacgcgtATGCAGAACAGTCACAGC | ggtagaattcacgcgtTTACTGTAATCTTGGCCAGTA |
| | Biosystems | 58016 | GGAGTGAATC (SEQ ID NO. 1) | TTGAGACATATCAG (SEQ ID NO. 2) |
| LHX2 | Open | MHS1010- | cccaggtcccacgcgtATGCTGTTCCACAGTCTGT | ggtagaattcacgcgtTCATTAGAAAAGGTTGGTAAG |
| | Biosystems | 98053323 | CGGGC (SEQ ID NO. 3) | AGTCGTTTGTGAG (SEQ ID NO. 4) |
| OTX2 | Origene | SC111661 | cccaggtcccacgcgtATGATGTCTTATCTTAAGC | ggtagaattcacgcgtTCACAAAACCTGGAATTTCCA |
| | | | AACCGCCTTACG (SEQ ID NO. 5) | CGAGGATG (SEQ ID NO. 6) |
| SOX9 | Open | MHS1010- | cccaggtcccacgcgtATGAATCTCCTGGACCCCT | ggtagaattcacgcgtTCAAGGTCGAGTGAGCTGTGT |
| | Biosystems | 9205725 | TCATGAAG (SEQ ID NO. 7) | GTAG (SEQ ID NO. 8) |
| MITE | Open | MHS1010- | cccaggtcccacgcgtATGCTGGAAATGCTAGAA | ggtagaattcacgcgtTCAGTGACACCGACGGGAGAA |
| | Biosystems | 9206145 | TATAATCACTATCAGG (SEQ ID NO. 9) | AGG (SEQ ID NO. 10) |
| SIX3 | DF/HCC | HsCD0034 | cccaggtcccacgcgtATGGTATTCCGCTCCCCCC | ggtagaattcacgcgtTTATACATCACATTCCGAGTC |
| | | 8161 | TAGAC (SEQ ID NO. 11) | GCTGGAG (SEQ ID NO. 12) |
| ZNF92 | Open | MHS1010- | cccaggtcccacgcgtATGGGACCACTGACATTT | ggtagaattcacgcgtTCAGGTTTGTAGTTTCTCTTT |
| | Biosystems | 9203746 | AGGGATGTG (SEQ ID NO. 13) | AGTATAATTTGAGG (SEQ ID NO. 14) |
| GLIS3 | Open | MHS1010- | cccaggtcccacgcgtATGATGGTTCAGCGACTG | ggtagaattcacgcgtTTAGCCTTCGGTGTAGACAGA |
| | Biosystems | 7295874 | GGACTCATTTC (SEQ ID NO. 15) | GGAGAG (SEQ ID NO. 16) |
| FOXD1 | DF/HCC | HsCD0029 | cccaggtcccacgcgtATGACCCTGAGCACTGAG | ggtagaattcacgcgtCTAACAATTGGAAATCCTAGC |
| | | 5189 | ATGTCCG (SEQ ID NO. 17) | AGTAAAGTTCTCG (SEQ ID NO. 18) |

### Viral Preparation and Transduction of HFF

For ectopic expression experiments, HFF were first infected with pLVX-Tet-On Advanced, expressing rtTA Advanced. Cells were grown in 1 mg/ml Geneticin® Selective Antibiotic (Life Technologies, cat. #10131035) for two weeks to select for cells harboring the plasmid.

For virus preparation, replication-incompetent lentiviral particles were packaged in 293T cells in the presence of the envelope, pMD2, and packaging, psPAX, plasmids. Viral supernatants from cultures 36, 48, 60 and 72 hours post-transfection were filtered through a 0.45 μM filter. High-titer virus preparations for all nine transcription factors were then added to HFF in the presence of 5 μg/ml of polybrene (day 1). A second transduction with virus for all nine factors was performed the next day (day 2). After two days, transduced HFF were split and transferred to iRPE growth medium (see below)(day 3). The following day iRPE medium was supplemented with 2 mg/ml doxycycline (Sigma Aldrich, cat. #D9891) (day 4). Medium was replaced every 3 days and fresh doxycycline added with every medium replacement.

iRPE Growth Conditions

iRPE lines were plated on Matrigel Basement Membrane Matrix-coated plates (BD, Cat. #CB-40234). iRPE cells were grown Minimum Essential Medium Eagle Alpha Modification (Sigma Aldrich, cat. #M4526) base medium containing 5% of Tet System Approved Fetal bovine serum (Clontech, cat. #631101), 1×N1 Medium Supplement (Sigma Aldrich, cat. #N6530), 1% Sodium Pyruvate (Life Technologies, cat. #11360070), 2 mM L-Glutamine (Life Technologies, cat. #25030-081), 1×MEM Non-Essential Amino Acids (Life Technologies, cat. #11140), 1 mg/ml Geneticin® Selective Antibiotic (Life Technologies, cat #10131035), 100 U/ml penicillin-streptomycin (Life Technologies, cat. #15140-163) and THT (20 μg/L hydrocortisone (Sigma Aldrich, cat. #H6909). 250 mg/L taurine (Sigma Aldrich, cat. #T0625), and 0.013 μm/L triiodothyronine (Sigma Aldrich, cat. #T2877). Cells were incubated in a 37° C., 5% CO₂ humidified incubator.

### Genotyping

To perform the genotyping of the iRPE lines, cells were lysed and genomic DNA was purified by treating samples with proteinase K, RNase A and phenol-chloroform extraction. DNA was amplified using GoTaq® Green Master Mix (Promega, cat. # M7122) using primers listed in Table 8. Primers were selected so one would hybridize in the coding region of the cDNA and the other would hybridize in the integrated viral sequence.

TABLE 8
Genotyping primers

| Factor | Forward primer | Reverse primer |
|---|---|---|
| PAX6 | P_TIGHT F1: 5′-GGGACAGCAGAGATCCAGTT-3′ | PAX6R: 5′-TACTACCACCGATTGCCCTG-3′ |
| | (SEQ ID NO. 19) | (SEQ ID NO. 20) |
| LHX2 | LHX2 F: 5-CTTTGCCATTAACCACAACC-3 | P_TIGHTR1: 5′-CTTCCTGACTAGGGGAGGAG-3′ |
| | (SEQ ID NO. 21) | (SEQ ID NO. 22) |
| OTX2 | P_TIGHT F1: 5′-GGGACAGCAGAGATCCAGTT-3′ | OTX2R: 5′-TGTCAGGGTAGCGAGTCTTG-3′ |
| | (SEQ ID NO. 19) | (SEQ ID NO. 23) |
| SOX9 | SOX9F: 5′-TCAACCTCCCACACTACAGC-3′ | P_TIGHTR2: 5′-AGACTGCCTTGGGAAAAGC-3′ |
| | (SEQ ID NO. 24) | (SEQ ID NO. 25) |
| MITF | P_TIGHT F1: 5′-GGGACAGCAGAGATCCAGTT-3′ | MITFR: 5′-CTCTCTGCCCTGTTTTGCTC-3′ |
| | (SEQ ID NO. 19) | (SEQ ID NO. 26) |
| SIX3 | SIX3F:5′-TCACTCCCACACAAGTAGGC-3′ | P-TIGHTR2: 5′-CTTCCATTTGTCACGTCCTG-3′ |
| | (SEQ ID NO. 27) | (SEQ ID NO. 28) |
| ZNF92 | P_TIGHT F1: 5′-GGGACAGCAGAGATCCAGTT-3′ | ZNF92R: 5′-TCTTGGGCAAAATGAGAACACA-3′ |
| | (SEQ ID NO. 19) | (SEQ ID NO. 29) |
| GLIS3 | P-TIGHT F3: 5′-AGGGACAGCAGAGATCCAGT-3′ | GLIS3R: 5′-GGGACCTGGTATCTGAAGGA-3′ |
| | (SEQ ID NO. 30) | (SEQ ID NO. 31) |
| FOXD1 | P_TIGHT F2: 5′-GGTACAGTGCAGGGGAAAGA-3′ | FOXD1R: AGAGGCATCGGACATCTCAG |
| | (SEQ ID NO. 32) | (SEQ ID NO. 33) |

### Immunostaining and Imaging

For immunostaining analysis, cells were grown in Corning® Transwell® polyester membrane cell culture inserts (Sigma Aldrich, cat. # CLS3460) for eight weeks in iRPE medium supplemented with 2 mg/ml doxycycline (Sigma Aldrich, cat. #D9891). Medium was replaced every three days. Cells plated in transwells were fixed in 4% paraformaldehyde for fifteen minutes on both apical and basal sides. Transwells inserts were then washed with ix PBS three times for five minutes. A 2 mm biopsy punch of the transwell membrane was transferred to a glass slide. Slides were incubated in blocking/permeabilizing solution (1% BSA, 1% saponin and 5% normal goat serum in 1×PBS) for one hour at room temperature. Subsequently, primary antibodies were diluted in blocking/permeabilizing solution and incubated on the slides overnight at 4° C. After three five-minute washes with 1×PBS, slides were incubated for one hour with appropriate Alexa secondary antibodies, diluted 1:500 in blocking/permeabilizing solution containing DAPI. Slides were then washed three times with 1×PBS and mounted with Prolong Gold Antifade Mountant (Life Technologies, cat. #P36930). Slides were left overnight at room temperature to solidify. Slides were visualized under a fluorescence microscope (Zeiss Axio Observer D1). Primary antibodies used for staining are listed in Table 5.

### Phagocytosis Assay

Rod outer segments (ROS) were isolated following previously described protocols (Ryeom et al., 1996). Retinas were dissected immediately following sacrifice from 25 mice, ROS were isolated, and approximately 1.0×10⁴ ROS were added to the supernatant of confluent cell cultures in transwells. The cells were then incubated for two hours at 37° C. Transwells were then washed 4-5 times with phosphate-buffered saline to remove all unbound ROS before fixation. Each transwell was fixed and immunostained for rhodopsin and dapi. Images were taken using fluorescence microscopy at a 40× magnification.

### Transepithelial Electrical Resistance (TER)

iRPE cells were grown in Corning® Transwell® polyester membrane cell culture inserts (Sigma Aldrich, cat. # CLS3460) for eight weeks in iRPE medium supplemented with 2 mg/ml doxycycline (Sigma Aldrich, cat. #D9891). Medium was replaced every 3 days. Resistance was measured using the EVOM Epithelial Voltohmmeter (World Precision Instruments).

### VEGF-A Release

iRPE cell and RPE cells (Salero et al., 2012) were grown in Corning® Transwell® polyester membrane cell culture inserts (Sigma Aldrich, cat. # CLS3460) for eight weeks in iRPE medium supplemented with 2 mg/ml doxycycline (Sigma Aldrich, cat. #D9891). Medium was replaced every three days with fresh doxycycline. Conditioned medium from apical and basal chambers of the same transwell insert was collected twenty-four hours following a complete medium change. VEGF-A protein secretion in conditioned medium was measured using a Human VEGF ELISA kit (Life Technologies, cat. #KHG0111), following the manufacturer's suggested protocol. Optical densities (450 nm) were measured within two hours, using a microplate reader (Perkin Elmer 1420 Multilabel Counter). Data was analyzed using GraphPad Prism 6.

### Transplantation

To study the ability of iRPE to integrate into the native retina we have performed subretinal transplantations into the wild-type rat retina. All animal experiments were performed according to the guidelines of the Association for Research in Vision and Ophthalmology. Three-week old albino Sprague-Dawley rats (Taconic) were used in these experiments. One day before the surgery all animals were switched to Cyclosporine A-supplemented water (210 mg/L) and remained on immunosuppressive treatment till the end of the study. One group of iRPE-transplanted animals also received Doxycycline in the water.

For the surgery, animals were anesthetized by intraperitoneal injection of ketamine/xylazine. Topical proparacaine (anesthetic) and tropicamide (mydriatic agent) drops were applied.

The subretinal injection was performed in one eye per animal using a 50 μm beveled glass needle, connected to a 10 μl Hamilton syringe through polyethylene tubing. The success of the injection and lack of complications (hemorrhage, retinotomy, leakage of cells into the vitreous) was assessed by fundus examination. Antibiotic ointment was applied to the eye for recovery.

Experimental groups were as follows: iRPE with Doxocycline treatment (n=5), iRPE without doxycycline treatment (n=5), hRPE (n=5) as positive control, vehicle injection (n=5) and non-injected eyes (n=5) as negative controls—5 groups total.

Two weeks after the injection animals were euthanized by CO2 inhalation, eyes were enucleated and fixed in alcohol fixative (Excalibur pathology), embedded in paraffin and sectioned.

### Accession Numbers

Raw and processed sequencing and microarray data were deposited in GEO (Gene Expression Omnibus; www.ncbi.nlm.nih.gov/geo/), under accession numbers GSE60024 and GSE64264 (reviewer link: www.ncbi.nlm.nih.gov/geo/query/acc.cgi?token=ihklqeqivdydnmh&acc=GSE64264).

### Microarray Expression Analysis for Knockdown Experiments

The raw data was obtained by using Affymetrix Gene Chip Operating Software using default settings. A Primeview CDF provided by Affymetrix was used to generate .CEL files. The CEL files were processed with the expresso command to convert the raw probe intensities to probeset expression values with MAS5 normalization using the standard tools available within the affy package in R. We used a loess regression (loess.normalize) from the affy package in R to renormalize the probe values using only the probes mapped to ribosomal genes to fit the loess. For genes with multiple probesets, the probeset with the maximum signal across experiments was selected for further analysis. Differential gene expression was determined using moderated t-statistic in the “limma” package (bioinfwehi.edu.au/limma/) from Bioconductor (www.bioconductor.org) (Smyth, 2004). Two independent hairpins were treated as replicates and compared to the two control hairpins. A gene was considered differentially expressed if it met the following criteria: 1) absolute log 2 fold-change≧1 between the mean expression of the two control shRNAs and the mean expression of the two target shRNAs, 2) adjusted p-value≦0.1 by a moderated t-test within the limma package with BH multiple hypothesis testing correction. Expression change of all RefSeq genes after shRNA knockdown in RPE cells is shown in Table 4. Raw data and processed gene expression tables can be found online associated with the raw and processed sequencing and microarray data were deposited in GEO under accession numbers GSE60024 and GSE64264 (www.ncbi.nlm.nih.gov/geo/).

### Determining Enriched GO Terms

The nature of differentially expressed genes was examined using GO analysis. Enriched Gene Ontology classification terms were identified using GO Term finder (go.princeton.edu/cgi-bin/GOTermFinder). The differentially up- and down-regulated genes from different candidate master transcription factor knockdown experiments were pooled together and used as inputs. The default settings of hypergeometric test with multiple hypothesis Bonferroni correction (adjusted p-Values of 0.01) was used.

### Gene Set Enrichment Analysis (GSEA)

GSEA (Broad Institute, www.broadinstitute.org/gsea/) was performed for differentiated expressed genes pooled from different candidate master transcription factor knockdown experiments. The differentially expressed genes were pre-ranked by the average fold change (log 2) in cells harboring transcription factor knockdown constructs relative to cells harboring the non-targeting shRNA control. The published RPE signature genes (Strunnikova et al., 2010) were used as the gene set for enrichment analysis.

### Illumina Sequencing and Library Generation

Purified ChIP DNA was used to prepare Illumina multiplexed sequencing libraries. Libraries for Illumina sequencing were prepared following the Illumina TruSeq DNA Sample Preparation v2 kit protocol with the following exceptions. After end-repair and A-tailing, immunoprecipitated DNA (˜10-50 ng) or input DNA (50 ng) was ligated with a 1:50 dilution of Illumina Adaptor Oligo Mix assigning one of 24 unique index primer sets in the kit to each sample. Following ligation, libraries were amplified by 18 cycles of PCR using the HiFi NGS Library Amplification kit from KAPA Biosystems. Amplified libraries were then size-selected using a 2% gel cassette in the Pippin Prep system from Sage Science set to capture fragments between 200 and 400 bp. Libraries were quantified by qPCR using the KAPA Biosystems Illumina Library Quantification kit according to kit protocols. Libraries with distinct TruSeq index primers were multiplexed by mixing at equimolar ratios and running together in a lane on the Illumina HiSeq 2000 for 40 bases in single read mode.

### Assigning Genes to Transcription Factor Binding Sites

All analyses were performed using RefSeq (GRCh37/hg19) human gene annotations. A gene was defined as transcribed if an enriched region for H3K27ac or RNA Pol II was located at the TSS. Active genes were assigned to transcription binding sites using the following method. Using a simple proximity rule, for each ChIP enriched region, the nearest TSS of an active gene was assigned to the region. Since promoters and distal elements can engage in looping interactions beyond the nearest genes (Sanyal et al., 2012), additional genes were assigned to ChIP enriched regions by using the distal DHS-to-promoter connection maps from a recent large-scale ENCODE study of promoters and their co-regulated distal DHS in 79 human cell types (Thurman et al., 2012). For each ChIP enriched region overlapping with a distal DHS in the distal DHS-to-promoter connection map, the genes from the DHS-to-promoter pair were assigned to the region.

### Definition of Active Enhancers

Active enhancers were defined as regions showing enrichment for H3K27Ac outside of promoters (greater than 2.5 kb away from any TSS). H3K27Ac is a histone modification associated with active enhancers (Creyghton et al., 2010b; Rada-Iglesias et al., 2010).

### Identifying Super-Enhancers

The identification of super-enhancers has been described in detail (Loven et al., 2013; Whyte et al., 2013; Hnisz et al., 2013). Briefly, H3K27ac peaks were used to identify constituent enhancers. These were stitched if within 12.5 kb, and peaks fully contained within +/−2 kb from a TSS were excluded from stitching. H3K27ac signal (less input control) was used to rank enhancers by their enrichment. 670 super-enhancers were separated from typical enhancers as previously described (Loven et al., 2013; Whyte et al., 2013). Super-enhancers were assigned to active genes using the ROSE software package (www.younglab.wi.mit.edu/super_enhancer_code.html). The super-enhancers and their target genes are listed in Table 6.

Principal Component Analysis and Differential Expression Analysis for iRPE

All expression datasets used for this analysis were processed together to generate Affymetrix MAS5-normalized probe set values. We processed all CEL files by using the probe definition (“hgu133plus2cdf”) and the standard MAS5 normalization technique within the affy package in R to get probe set expression values. The probesets of the same gene were next collapsed into a single value to represent the gene by taking the values of the probeset with the maximum signal across experiments.

The top 25% genes with the largest coefficient of variation across all expression profiles were used for Principal Component Analysis (PCA). PCA was done using R and the package MADE4 (Culhane et al., 2005). Previously published microarray data used in PCA analysis is listed in Table 9.

Differential gene expression between human foreskin fibroblasts (HFF) and retinal pigment epithelial (RPE) cells was determined using moderated t-statistic in the “limma” package (bioinfwehi.edu.au/limma/) from Bioconductor (www.bioconductor.org) (Smyth, 2004). The differentially expressed genes were required to have absolute value of log 2 fold-change≧1 between the mean expression of HFFs and the mean expression RPEs, and FDR-adjusted p-value≦0.01. The heat map in FIG. 4F shows the fold change (log 2) of the differentially expressed genes relative to the mean expression of HFF.

### Previous Characterization of Transcription Factors

The extent of previous characterization of individual TFs was estimated by performing the following search on PubMed: HGNC gene name for transcription factor [Title/Abstract] AND transcription AND factor*. The GO annotations (Biological Process) for all transcription factors from the SMART database were downloaded at BioMart-Eensembl (www.ensembl.org/biomart) (Letunic et al., 2015). As noted, transcription factors were filtered for those with GO annotations supported by experimental evidence (evidence codes: EXP, IDA, IPI, IMP, IGI, or IEP).

### TF Expression Level Analysis

The expression levels of core TFs were compared to those of non-core TFs. The expression profiles were processed as described in the section, Identification of Candidate Core Transcription Factors. For each cell type, multiple microarrays were commonly available, so the expression level of a TF was calculated for each cell type by taking the median expression level across the set of microarrays for that cell type. For expression analysis, if a factor was called a candidate core factor in a cell type, the expression value of that factor in that cell type was selected and the total set of such values was used to analyze the expression of candidate core factors. All other expression values were used to analyze expression of non-core factors. The distribution of expression level of core and non-core TFs were displayed in a boxplot.

### DNA Binding Domain Analysis

The annotations of the DNA binding protein domains of all transcription factors from the SMART database were downloaded from BioMart-Ensembl.

### Conservation Analysis

For the genes that encode candidate core transcription factors, orthologues from multiple species were downloaded from BioMart-Eensembl (www.ensembl.org/biomart) (Letunic et al., 2015). The species are selected to represent primates (chimpanzee, macaque, orangutan), mammals (mouse, rat, pig, cow, dog, horse), vertebrate (opossum, platypus, fugu, tetraodon, stickleback, zebrafish, frog, chicken), metazoa (ciona, fly, worm), and eukaryotes (baker's yeast). The presence or absence of the orthologous genes in the selected species was displayed in a heatmap. The rows of the heatmap were ordered by k-mean clustering with number of clusters equal to 3.

### Comparisons to Super-Enhancer Associated TFs

We examined whether genes encoding candidate core TFs were commonly associated with super-enhancers. For cell types where we had both candidate core TF predictions and available H3K27Ac chromatin immunoprecipitation data, we used the H3K27Ac data to first identify super-enhancers and assign them to genes (Hnisz et al., 2013). For each cell type, all TFs were then ranked based on their expression-specificity scores as gene sets. GSEA pre-ranked enrichment analysis was next used to determine whether the super-enhancer associated TFs were enriched for transcription factors that have high expression-specificity scores. For comparisons, GSEA pre-ranked enrichment analysis was also performed on gene sets made from all transcription factors sorted on expression specificity scores from a random, non-matched cell type (embryonic stem cells).

Principal Component Analysis and Differential Expression Analysis for iRPE

All expression datasets used for this analysis were processed together to generate Affymetrix MAS5-normalized probe set values. We processed all CEL files by using the probe definition (“hgu133plus2cdf”) and the standard MAS5 normalization technique within the affy package in R to get probe set expression values. The probesets of the same gene were next collapsed into a single value to represent the gene by taking the values of the probeset with the maximum signal across experiments.

The top 25% genes with the largest coefficient of variation across all expression profiles were used for Principal Component Analysis (PCA). PCA was done using R and the package MADE4 (Culhane et al., 2005). Previously published microarray data used in PCA analysis is listed in Table 9.

Differential gene expression between human foreskin fibroblasts (HFF) and retinal pigment epithelial (RPE) cells was determined using moderated tstatistic in the “limma” package (bioinfwehi.edu.au/limma/) from Bioconductor (www.bioconductor.org) (Smyth, 2004). The differentially expressed genes were required to have absolute value of log 2 fold-change≧1 between the mean expression of HFFs and the mean expression RPEs, and FDR-adjusted p-value≦0.01.

### REFERENCES

Avilion, A. A., Nicolis, S. K., Pevny, L. H., Perez, L., Vivian, N., and Lovell-Badge, R. (2003). Multipotent cell lineages in early mouse development depend on SOX2 function. Genes Dev 17, 126-140.
Benayoun, B. A., Pollina, E. A., Ucar, D., Mahmoudi, S., Karra, K., Wong, E. D., Devarajan, K., Daugherty, A. C., Kundaje, A. B., Mancini, E., et al. (2014). H3K4me3 breadth is linked to cell identity and transcriptional consistency. Cell 158, 673-688.
Bharti, K., Gasper, M., Ou, J. X., Brucato, M., Clore-Gronenborn, K., Pickel, J., and Arnheiter, H. (2012). A Regulatory Loop Involving PAX6, MITF, and WNT Signaling Controls Retinal Pigment Epithelium Development. Plos Genetics 8.
Binder, S., Stanzel, B. V., Krebs, I., and Glittenberg, C. (2007). Transplantation of the RPE in AMD. Progress in retinal and eye research 26, 516-554.
Bok, D. (1993). The retinal pigment epithelium: a versatile partner in vision. Journal of cell science Supplement 17, 189-195.
Bok, D., and Hall, M. O. (1971). The role of the pigment epithelium in the etiology of inherited retinal dystrophy in the rat. The Journal of cell biology 49, 664-682.
Breitling, R., Armengaud, P., Amtmann, A., and Herzyk, P. (2004). Rank products: a simple, yet powerful, new method to detect differentially regulated genes in replicated microarray experiments. FEBS letters 573, 83-92.
Boyer, L. A., Lee, T. I., Cole, M. F., Johnstone, S. E., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
Buganim, Y., Faddah, D. A., and Jaenisch, R. (2013). Mechanisms and models of somatic cell reprogramming. Nature reviews Genetics 14, 427-439.
Buganim, Y., Itskovich, E., Hu, Y. C., Cheng, A. W., Ganz, K., Sarkar, S., Fu, D., Welstead, G. G., Page, D. C., and Jaenisch, R. (2012). Direct reprogramming of fibroblasts into embryonic Sertoli-like cells by defined factors. Cell Stem Cell 11, 373-386.
Cabili, M. N., Trapnell, C., Goff, L., Koziol, M., Tazon-Vega, B., Regev, A., and Rinn, J. L. (2011). Integrative annotation of human large intergenic noncoding RNAs reveals global properties and specific subclasses. Genes Dev 25, 1915-1927.
Cahan, P., Li, H., Morris, S. A., Lummertz da Rocha, E., Daley, G. Q., and Collins, J. J. (2014). CellNet: network biology applied to stem cell engineering. Cell 158, 903-915.
Chambers, I., Colby, D., Robertson, M., Nichols, J., Lee, S., Tweedie, S., and Smith, A. (2003). Functional expression cloning of Nanog, a pluripotency sustaining factor in embryonic stem cells. Cell 113, 643-655.
Chiba, C. (2014). The retinal pigment epithelium: an important player of retinal disorders and regeneration. Experimental eye research 123, 107-114.
Creyghton, M. P., Cheng, A. W., Welstead, G. G., Kooistra, T., Carey, B. W., Steine, E. J., Hanna, J., Lodato, M. A., Frampton, G. M., Sharp, P. A., et al. (2010). Histone H3K27ac separates active from poised enhancers and predicts developmental state. Proceedings of the National Academy of Sciences of the United States of America 107, 21931-21936.
Cyranoski, D. (2013). Stem cells cruise to clinic. Nature 494, 413.
Cyranoski, D. (2014). Stem-cell method faces fresh questions. Nature 507, 283.
da Cruz, L., Chen, F. K., Ahmado, A., Greenwood, J., and Coffey, P. (2007). RPE transplantation and its role in retinal disease. Progress in retinal and eye research 26, 598-635.
Farh, K. K., Marson, A., Zhu, J., Kleinewietfeld, M., Housley, W. J., Beik, S., Shoresh, N., Whitton, H., Ryan, R. J., Shishkin, A. A., et al. (2014). Genetic and epigenetic fine mapping of causal autoimmune disease variants. Nature.
Ford, K. M., Saint-Geniez, M., Walshe, T., Zahr, A., and D'Amore, P. A. (2011). Expression and role of VEGF in the adult retinal pigment epithelium. Investigative ophthalmology & visual science 52, 9478-9487.
Fuhrmann, S., Zou, C., and Levine, E. M. (2014). Retinal pigment epithelium development, plasticity, and tissue homeostasis. Experimental eye research 123, 141-150.
Fuglede, B., and Topsoe, F (2004). Jensen-Shannon Divergence and Hilbert space embedding. Information theory 31.
Graf, T. (2011). Historical origins of transdifferentiation and reprogramming. Cell Stem Cell 9, 504-516.
Graf, T., and Enver, T. (2009). Forcing cells to change lineages. Nature 462, 587-594.
Harhaj, N. S., and Antonetti, D. A. (2004). Regulation of tight junctions and loss of barrier function in pathophysiology. The international journal of biochemistry & cell biology 36, 1206-1237.
Henriques, T., Gilchrist, D. A., Nechaev, S., Bern, M., Muse, G. W., Burkholder, A., Fargo, D. C., and Adelman, K. (2013). Stable pausing by RNA polymerase II provides an opportunity to target and integrate regulatory signals. Molecular cell 52, 517-528.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Huang, P., He, Z., Ji, S., Sun, H, Xiang, D., Liu, C., Hu, Y., Wang, X., and Hui, L. (2011). Induction of functional hepatocyte-like cells from mouse fibroblasts by defined factors. Nature 475, 386-389.
Hwang, P. I., Wu, H. B., Wang, C. D., Lin, B. L., Chen, C. T., Yuan, S., Wu, G., and Li, K. C. (2011). Tissue-specific gene expression templates for accurate molecular characterization of the normal physiological states of multiple human tissues with implication in development and cancer studies. BMC genomics 12, 439.
Idelson, M., Alper, R., Obolensky, A., Ben-Shushan, E., Hemo, I., Yachimovich-Cohen, N, Khaner, H.,
Smith, Y., Wiser, O., Gropp, M., et al. (2009). Directed Differentiation of Human Embryonic Stem Cells into Functional Retinal Pigment Epithelium Cells. Cell Stem Cell 5, 396-408.
Iwafuchi-Doi, M., and Zaret, K. S. (2014). Pioneer transcription factors in cell reprogramming. Genes Dev 28, 2679-2692.
Ivanova, N., Dobrin, R., Lu, R., Kotenko, I., Levorse, J., DeCoste, C., Schafer, X., Lun, Y., and Lemischka, I. R. (2006). Dissecting self-renewal in stem cells with RNA interference. Nature 442, 533-538.
Kamao, H., Mandai, M., Okamoto, S., Sakai, N., Suga, A., Sugita, S., Kiryu, J., and Takahashi, M. (2014). Characterization of human induced pluripotent stem cell-derived retinal pigment epithelium cell sheets aiming for clinical application. Stem cell reports 2, 205-218.
Kim, J., Chu, J., Shen, X., Wang, J., and Orkin, S. H. (2008). An extended transcriptional network for pluripotency of embryonic stem cells. Cell 132, 1049-1061.
Lang, A. H., Li, H., Collins, J. J., and Mehta, P. (2014). Epigenetic landscapes explain partially reprogrammed cells and identify key reprogramming genes. PLoS computational biology 10, e1003734.
Lee, T. I., Johnstone, S. E., and Young, R. A. (2006). Chromatin immunoprecipitation and microarray-based analysis of protein location. Nature protocols 1, 729-748.
Lee, T. I., and Young, R. A. (2013). Transcriptional regulation and its misregulation in disease. Cell 152, 1237-1251.
Lim, L. S., Mitchell, P., Seddon, J. M., Holz, F. G., and Wong, T. Y. (2012). Age-related macular degeneration. Lancet 379, 1728-1738.
Lujan, E., Chanda, S., Ahlenius, H., Sudhof, T. C., and Wernig, M. (2012). Direct conversion of mouse fibroblasts to self-renewing, tripotent neural precursor cells. Proceedings of the National Academy of Sciences of the United States of America 109, 2527-2532.
Marson, A., Levine, S. S., Cole, M. F., Frampton, G. M., Brambrink, T., Johnstone, S., Guenther, M. G., Johnston, W. K., Wernig, M., Newman, J., et al. (2008). Connecting microRNA genes to the core transcriptional regulatory circuitry of embryonic stem cells. Cell 134, 521-533.
Maurano, M. T., Humbert, R., Rynes, E., Thurman, R. E., Haugen, E., Wang, H., Reynolds, A. P., Sandstrom, R., Qu, H. Z., Brody, J., et al. (2012). Systematic Localization of Common Disease-Associated Variation in Regulatory DNA. Science 337, 1190-1195.
Martinez-Morales, J. R., Dolez, V., Rodrigo, I., Zaccarini, R., Leconte, L., Bovolenta, P., and Saule, S. (2003). OTX2 activates the molecular network underlying retina pigment epithelium differentiation. Journal of Biological Chemistry 278, 21721-21731.
Masuda, T., and Esumi, N. (2010). SOX9, through Interaction with Microphthalmia-associated Transcription Factor (MITF) and OTX2, Regulates BEST1 Expression in the Retinal Pigment Epithelium. Journal of Biological Chemistry 285, 26933-26944.
Matsuo, I., Kuratani, S., Kimura, C., Takeda, N., and Aizawa, S. (1995). Mouse Otx2 Functions in the Formation and Patterning of Rostral Head. Genes & Development 9, 2646-2658.
Maurano, M. T., Humbert, R., Rynes, E., Thurman, R. E., Haugen, E., Wang, H., Reynolds, A. P., Sandstrom, R., Qu, H. Z., Brody, J., et al. (2012). Systematic Localization of Common Disease-Associated Variation in Regulatory DNA. Science 337, 1190-1195.
Morris, S. A., Cahan, P., Li, H., Zhao, A. M., San Roman, A. K., Shivdasani, R. A., Collins, J. J., and Daley, G. Q. (2014). Dissecting engineered cell types and enhancing cell fate conversion via CellNet. Cell 158, 889-902.
Morris, S. A., and Daley, G. Q. (2013). A blueprint for engineering cell fate: current technologies to reprogram cell identity. Cell Res 23, 33-48.
Nichols, J., Zevnik, B., Anastassiadis, K., Niwa, H., Klewe-Nebenius, D., Chambers, I., Scholer, H., and Smith, A. (1998). Formation of pluripotent stem cells in the mammalian embryo depends on the POU transcription factor Oct4. Cell 95, 379-391.
Novershtern, N., Subramanian, A., Lawton, L. N., Mak, R. H., Haining, W. N., McConkey, M. E., Habib, N., Yosef, N., Chang, C. Y., Shay, T., et al. (2011). Densely interconnected transcriptional circuits control cell states in human hematopoiesis. Cell 144, 296-309.
Odom, D. T., Dowell, R. D., Jacobsen, E. S., Nekludova, L., Rolfe, P. A., Danford, T. W., Gifford, D. K., Fraenkel, E., Bell, G. I., and Young, R. A. (2006). Core transcriptional regulatory circuitry in human hepatocytes. Mol Syst Biol 2, 2006 0017.
Parker, S. C., Stitzel, M. L., Taylor, D. L., Orozco, J. M., Erdos, M. R., Akiyama, J. A., van Bueren, K. L., Chines, P. S., Narisu, N., Program, N. C. S., et al. (2013). Chromatin stretch enhancer states drive cell-specific gene regulation and harbor human disease risk variants. Proceedings of the National Academy of Sciences of the United States of America 110, 17921-17926.
Rada-Iglesias, A., Bajpai, R., Swigut, T., Brugmann, S. A., Flynn, R. A., and Wysocka, J. (2011). A unique chromatin signature uncovers early developmental enhancers in humans. Nature 470, 279-283.
Rivera, C. M., and Ren, B. (2013). Mapping human epigenomes. Cell 155, 39-55.
Roost, M. S., van Iperen, L., Ariyurek, Y., Buermans, H. P., Arindrarto, W., Devalla, H. D., Passier, R., Mummery, C. L., Carlotti, F., de Koning, E. J., et al. (2015). KeyGenes, a Tool to Probe Tissue Differentiation Using a Human Fetal Transcriptional Atlas. Stem cell reports 4, 1112-1124.
Ryeom, S. W., Sparrow, J. R., and Silverstein, R. L. (1996). CD36 participates in the phagocytosis of rod outer segments by retinal pigment epithelium. Journal of cell science 109 (Pt 2), 387-395.
Saint-Geniez, M., Kurihara, T., Sekiyama, E., Maldonado, A. E., and D'Amore, P. A. (2009). An essential role for RPE-derived soluble VEGF in the maintenance of the choriocapillaris. Proceedings of the National Academy of Sciences of the United States of America 106, 18751-18756.
Salero, E., Blenkinsop, T. A., Corneo, B., Harris, A., Rabin, D., Stern, J. H., and Temple, S. (2012). Adult human RPE can be activated into a multipotent stem cell that produces mesenchymal derivatives. Cell Stem Cell 10, 88-95.
Sancho-Martinez, I., Baek, S. H., and Izpisua Belmonte, J. C. (2012). Lineage conversion methodologies meet the reprogramming toolbox. Nat Cell Biol 14, 892-899.
Sanda, T., Lawton, L. N., Barrasa, M. I., Fan, Z. P., Kohlhammer, H., Gutierrez, A., Ma, W., Tatarek, J., Ahn, Y., Kelliher, M. A., et al. (2012). Core transcriptional regulatory circuit controlled by the TALI complex in human T cell acute lymphoblastic leukemia. Cancer Cell 22, 209-221.
Schwartz, S. D., Hubschman, J. P., Heilwell, G., Franco-Cardenas, V., Pan, C. K., Ostrick, R. M., Mickunas, E., Gay, R., Klimanskaya, I., and Lanza, R. (2012). Embryonic stem cell trials for macular degeneration: a preliminary report. Lancet 379, 713-720.
Schwartz, S. D., Regillo, C. D., Lam, B. L., Eliott, D., Rosenfeld, P. J., Gregori, N. Z., Hubschman, J. P., Davis, J. L., Heilwell, G., Spirn, M., et al. (2014). Human embryonic stem cell-derived retinal pigment epithelium in patients with age-related macular degeneration and Stargardt's macular dystrophy: follow-up of two open-label phase 1/2 studies. Lancet.
Sheng, C., Zheng, Q., Wu, J., Xu, Z., Wang, L., Li, W., Zhang, H., Zhao, X. Y., Liu, L., Wang, Z., et al. (2012). Direct reprogramming of Sertoli cells into multipotent neural stem cells by defined factors. Cell Res 22, 208-218.
Soufi, A., Donahue, G., and Zaret, K. S. (2012). Facilitators and impediments of the pluripotency reprogramming factors' initial engagement with the genome. Cell 151, 994-1004.
Sparrow, J. R., Hicks, D., and Hamel, C. P. (2010). The retinal pigment epithelium in health and disease. Curr Mol Med 10, 802-823.
Stergachis, A. B., Neph, S., Reynolds, A., Humbert, R., Miller, B., Paige, S. L., Vernot, B., Cheng, J. B., Thurman, R. E., Sandstrom, R., et al. (2013). Developmental fate and cellular maturity encoded in human regulatory DNA landscapes. Cell 154, 888-903.
Stevenson, B. R., Siliciano, J. D., Mooseker, M. S., and Goodenough, D. A. (1986). Identification of ZO-1: a high molecular weight polypeptide associated with the tight junction (zonula occludens) in a variety of epithelia. The Journal of cell biology 103, 755-766.
Strauss, O. (2005). The retinal pigment epithelium in visual function. Physiological Reviews 85, 845-881.
Strunnikova, N. V., Maminishkis, A., Barb, J. J., Wang, F., Zhi, C., Sergeev, Y., Chen, W., Edwards, A. O., Stambolian, D., Abecasis, G., et al. (2010). Transcriptome analysis and molecular signature of human retinal pigment epithelium. Hum Mol Genet 19, 2468-2486.
Tapscott, S. J., Davis, R. L., Thayer, M. J., Cheng, P. F., Weintraub, H., and Lassar, A. B. (1988). MyoD1: a nuclear phosphoprotein requiring a Myc homology region to convert fibroblasts to myoblasts. Science 242, 405-411.
Vaquerizas, J. M., Kummerfeld, S. K., Teichmann, S. A., and Luscombe, N. M. (2009). A census of human transcription factors: function, expression and evolution. Nature reviews Genetics 10, 252-263.
Vierbuchen, T., Ostermeier, A., Pang, Z. P., Kokubu, Y., Sudhof, T. C., and Wernig, M. (2010). Direct conversion of fibroblasts to functional neurons by defined factors. Nature 463, 1035-1041.
Vierbuchen, T., and Wernig, M. (2012). Molecular roadblocks for cellular reprogramming. Molecular cell 47, 827-838.
Wang, Z. X., Kueh, J. L., Teh, C. H., Rossbach, M., Lim, L., Li, P., Wong, K. Y., Lufkin, T., Robson, P., and Stanton, L. W. (2007a). Zfp206 is a transcription factor that controls pluripotency of embryonic stem cells. Stem Cells 25, 2173-2182.
Wang, Z. X., Teh, C. H., Kueh, J. L., Lufkin, T., Robson, P., and Stanton, L. W. (2007b). Oct4 and Sox2 directly regulate expression of another pluripotency transcription factor, Zfp206, in embryonic stem cells. J Biol Chem 282, 12822-12830.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Xie, W., and Ren, B. (2013). Developmental biology Enhancing pluripotency and lineage specification. Science 341, 245-247.
Yamanaka, S. (2012). Induced pluripotent stem cells: past, present, and future. Cell Stem Cell 10, 678-684.
Zhang, K., Liu, G. H., Yi, F., Montserrat, N., Hishida, T., Esteban, C. R., and Izpisua Belmonte, J. C. (2014). Direct conversion of human fibroblasts into retinal pigment epithelium-like cells by defined factors. Protein Cell 5, 48-58.
Zhou, J. X., Brusch, L., and Huang, S. (2011). Predicting pancreas cell fate decisions and reprogramming with a hierarchical multi-attractor model. PloS one 6, e14752.
Ziller, M. J., Edri, R., Yaffe, Y., Donaghey, J., Pop, R., Mallard, W., Issner, R., Gifford, C. A., Goren, A., Xing, J., et al. (2015). Dissecting neural differentiation regulatory networks through epigenetic footprinting. Nature 518, 355-359.
Breitling, R., Armengaud, P., Amtmann, A., and Herzyk, P. (2004). Rank products: a simple, yet powerful, new method to detect differentially regulated genes in replicated microarray experiments. FEBS letters 573, 83-92.
Burglin, T. R. (2011). Homeodomain subtypes and functional diversity. Sub-cellular biochemistry 52, 95-122.
Culhane, A. C., Thioulouse, J., Perriere, G., and Higgins, D. G. (2005). MADE4: an R package for multivariate analysis of gene expression data. Bioinformatics 21, 2789-2790.
Fuglede, B., and Topsoe, F (2004). Jensen-Shannon Divergence and Hilbert space embedding. Information theory 31.
Guo, J., Hammar, M., Oberg, L., Padmanabhuni, S. S., Bjareland, M., and Dalevi, D. (2013). Combining evidence of preferential gene-tissue relationships from multiple sources. PloS one 8, e70568.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Holmes, M. L., Huntington, N. D., Thong, R. P., Brady, J., Hayakawa, Y., Andoniou, C. E., Fleming, P., Shi, W., Smyth, G. K., Degli-Esposti, M. A., et al. (2014). Peripheral natural killer cell maturation depends on the transcription factor Aiolos. EMBO J 33, 2721-2734.
Langmead, B., Trapnell, C., Pop, M., and Salzberg, S. L. (2009). Ultrafast and memory-efficient alignment of short DNA sequences to the human genome. Genome biology 10, R25.
Lee, T. I., Johnstone, S. E., and Young, R. A. (2006). Chromatin immunoprecipitation and microarray-based analysis of protein location. Nature protocols 1, 729-748.
Loven, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of super-enhancers. Cell 153, 320-334.
Letunic, I., Doerks, T., and Bork, P. (2015). SMART: recent updates, new developments and status in 2015. Nucleic acids research 43, D257-260.
Luscombe, N. M., Austin, S. E., Berman, H. M., and Thornton, J. M. (2000). An overview of the structures of protein-DNA complexes. Genome biology 1, REVIEWS001.
Parker, S. C., Stitzel, M. L., Taylor, D. L., Orozco, J. M., Erdos, M. R., Akiyama, J. A., van Bueren, K. L., Chines, P. S., Narisu, N., Program, N. C. S., et al. (2013). Chromatin stretch enhancer states drive cell-specific gene regulation and harbor human disease risk variants. Proceedings of the National Academy of Sciences of the United States of America 110, 17921-17926.
Sanyal, A., Lajoie, B. R., Jain, G., and Dekker, J. (2012). The long-range interaction landscape of gene promoters. Nature 489, 109-113.
Smyth, G. K. (2004). Linear models and empirical bayes methods for assessing differential expression in microarray experiments. Statistical applications in genetics and molecular biology 3, Article 3.
Strunnikova, N. V., Maminishkis, A., Barb, J. J., Wang, F., Zhi, C., Sergeev, Y., Chen, W., Edwards, A. O., Stambolian, D., Abecasis, G., et al. (2010). Transcriptome analysis and molecular signature of human retinal pigment epithelium. Hum Mol Genet 19, 2468-2486.
Thurman, R. E., Rynes, E., Humbert, R., Vierstra, J., Maurano, M. T., Haugen, E., Sheffield, N. C., Stergachis, A. B., Wang, H., Vernot, B., et al. (2012). The accessible chromatin landscape of the human genome. Nature 489, 75-82.
Vaquerizas, J. M., Kummerfeld, S. K., Teichmann, S. A., and Luscombe, N. M. (2009). A census of human transcription factors: function, expression and evolution. Nature reviews Genetics 10, 252-263.
Wapinski, O. L., Vierbuchen, T., Qu, K., Lee, Q. Y., Chanda, S., Fuentes, D. R., Giresi, P. G., Ng, Y. H., Marro, S., Neff, N. F., et al. (2013). Hierarchical mechanisms for direct reprogramming of fibroblasts to neurons. Cell 155, 621-635.
Weirauch, M. T., and Hughes, T. R. (2011). A catalogue of eukaryotic transcription factor types, their evolutionary origin, and species distribution. Sub-cellular biochemistry 52, 25-73.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Xiang, C., Baubet, V., Pal, S., Holderbaum, L., Tatard, V., Jiang, P., Davuluri, R. V., and Dahmane, N. (2012). RP58/ZNF238 directly modulates proneurogenic gene levels and is required for neuronal differentiation and brain expansion. Cell death and differentiation 19, 692-702.
Xu, J., Du, Y., and Deng, H. (2015). Direct lineage reprogramming: strategies, mechanisms, and applications. Cell Stem Cell 16, 119-134.
Zhang, X., Zhang, R., Jiang, Y., Sun, P., Tang, G., Wang, X., Lv, H., and Li, X. (2011). The expanded human disease network combining protein-protein interaction information. European journal of human genetics: EJHG 19, 783-788.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome biology 9, R137.

1. A method of identifying master transcription factors of a query cell type, comprising:
providing gene expression data of a plurality of transcription factors for a query cell type;
relatively quantifying expression level and expression specificity of each transcription factor in the query cell type against a background gene expression profile assembled from a collection of cell types by using an entropy-based measure of Jensen-Shannon divergence (JSD), thereby generating a cell-type-specificity score for each transcription factor; and
ranking the plurality of transcription factors based on their corresponding cell-type-specificity scores, wherein top ranked transcription factors are identified as master transcription factors of the query cell type.

2. The method of claim 1, wherein in the providing step, the gene expression data is selected from one or more of: gene expression profiling by microarray or sequencing, non-coding RNA profiling by microarray or sequencing, chromatin immunoprecipitation profiling by microarray or sequencing, genome methylation profiling by microarray or sequencing, genome variation profiling by array, single nucleotide polymorphism array, serial analysis of gene expression, and/or protein array.

3. The method of claim 1, wherein in the providing step, a plurality of disparate sets of gene expression data are provided.

4. The method of claim 3, further comprising comparing the plurality of disparate sets of gene expression data by pair-wise Pearson correlation, grouping the plurality of disparate sets into subclusters using hierarchical clustering, analyzing the subclusters in a modular fashion, and removing subclusters consisting of data sets that have Pearson correlation coefficients less than 0.7 compared to other data sets.

5. The method of claim 4, wherein the ranking step further comprises calculating rank product-based scores for each set of gene expression data that is retained after the removing step.

6. The method of claim 1, wherein the quantifying step uses an algorithm which:
assumes an idealized pattern where an ideal master transcription factor is expressed to a high level in the query cell type and not expressed in any other cell type;
compares the observed pattern of an actual transcription factor with the idealized pattern; and
generates the cell-type-specificity score based on how well the observed pattern matches with the idealized pattern.

7. The method of claim 6, further comprising:
creating two same-sized, discrete, first and second probability vectors to represent the observed pattern and the ideal pattern, respectively; wherein for the observed pattern, the first probability vector is formed by values from the gene expression data of the query cell type and the background gene expression profile, and elements in the first probability vector are divided by the sum of the elements so that the normalized vector sums to 1;
wherein for the idealized pattern, the second probability vector is formed by a value of 1 at a position equivalent to that of the query cell type and zeroes at all other positions; and
calculating a distance metric between the first and second vectors using JSD, thereby generating the cell-type-specificity score.

8. The method of claim 1, wherein the background gene expression profile is prepared by a method comprising the steps of:
collecting a background dataset comprising expression datasets of different cell and tissues types,
normalizing expression profiles of the expression datasets, and
balancing the background dataset.

9. The method of claim 8, wherein in the collecting step, the expression datasets are gathered from Human Body Index collection of expression datasets.

10. The method of claim 8, wherein in the normalizing step, the expression profiles are processed and normalized to generate Affymetrix MAS5-normalized probe set values.

11. The method of claim 8, wherein the balancing step comprises clustering the expression profiles in the background dataset by similarity, and choosing from clusters of highly similar expression profiles a single representative profile while removing other profiles from the background dataset.

12. The method of claim 1, wherein top 20 or less ranked transcription factors are identified as master transcription factors of the query cell type.

13. The method of claim 1, wherein top 10 or less ranked transcription factors are identified as master transcription factors of the query cell type.

14. The method of claim 1, wherein top 5 or less ranked transcription factors are identified as master transcription factors of the query cell type.

15. A method of transdifferentiating a somatic cell into an induced retinal pigment epithelium (iRPE) cell, comprising increasing expression of at least four of PAX6, LHX2, OTX2, SOX9, MITF, SIX3, ZNF92, GLIS3, C11orf9 and FOXD1, or a variant of any one or more of the foregoing, in a somatic cell that is not retinal pigment epithelium cell.

16. The method of claim 15, further comprising ectopically expressing OTX2, SIX3, GLIS3, and at least one of PAX6, LHX2, SOX9, MITF, ZNF92, C11orf9 and FOXD1, or a variant of any one or more of the foregoing in the somatic cell.

17. The method of claim 15, comprising increasing expression of PAX6, OTX2, MITF, SIX3, GLIS3 and FOXD1, or a variant of any one or more of the foregoing.

18. An induced retinal pigment epithelium (iRPE) cell, comprising at least four of ectopically expressed PAX6, LHX2, OTX2, SOX9, MITF, SIX3, ZNF92, GLIS3, C11orf9 and FOXD1, or a variant of any one or more of the foregoing, in a somatic cell that is not retinal pigment epithelium cell.

19. The induced iRPE of claim 18, comprising ectopically expressed OTX2, SIX3, GLIS3, and at least one of PAX6, LHX2, SOX9, MITF, ZNF92, C11orf9 and FOXD1, or a variant of any one or more of the foregoing.

20. The induced iRPE of claim 18, comprising ectopically expressed PAX6, OTX2, MITF, SIX3, GLIS3 and FOXD1, or a variant of any one or more of the foregoing.
